(19) United States
(12) Patent Application Publication
(10) Pub. No.: US 2022/0226543 A1
(43) Pub. Date: Jul. 21, 2022

(54) Patterned Hydrogel Devices and Methods for Neural Regeneration

(71) Applicants: Mayo Foundation for Medical Education and Research, Rochester, MN (US); The Trustees of Princeton University, Princeton, NJ (US)

(72) Inventors: Michael J. Yaszemski, Rochester, MN (US); Anthony J. Windebank, Zumbro Falls, MN (US); Ahad M. Siddiqui, Rochester, MN (US); James L. Herrick, Rochester, MN (US); Suzanne L. Glass, Winston Salem, NC (US); Alan L. Miller, II, Rochester, MN (US); Brian E. Waletzki, Eyota, MN (US); Nicolas N. Madigan, Rochester, MN (US); Jeffrey Schwartz, Princeton, NJ (US); Jean E. Schwarzbauer, Princeton, NJ (US); Kelly S. Lim, Princeton, NJ (US); Stephen B. Bandini, Princeton, NJ (US); Gregory M. Harris, Princeton, NJ (US); Jeffrey W. Chen, Princeton, NJ (US)

(21) Appl. No.: 17/607,245

(22) PCT Filed: Apr. 30, 2020
(86) PCT No.: PCT/US2020/030740

§ 371 Date: Oct. 28, 2021

Related U.S. Application Data
(60) Provisional application No. 62/840,504, filed on Apr. 30, 2019.

(51) Int. Cl. A61L 27/52; A61K 38/18; A61K 35/28; A61K 35/30; A61L 27/16; A61L 27/06; A61L 27/38; A61L 27/28; A61L 27/04

(52) U.S. Cl. CPC A61L 27/52; A61K 38/185; A61K 35/28; A61K 35/30; A61L 27/16; A61L 2430/32; A61L 27/383; A61L 27/3834; A61L 27/28; A61L 27/047; A61L 27/06

(57) ABSTRACT

A matrix for neuron regeneration. The matrix can include a sheet having a first surface and a second surface opposite the first surface, the second surface having a plurality of integrally formed ridges. The sheet can have a spiral shape, such that the first surface of the sheet faces the second surface of the sheet. The sheet and the integrally formed ridges can comprise oligo(poly(ethylene glycol) fumarate).

### CROSS-REFERENCE TO RELATED APPLICATIONS

The present application is based on and claims priority to U.S. Provisional Patent Application No. 62/840,504, filed Apr. 30, 2019, and entitled “Patterned Hydrogel Devices for Neural Regeneration,” which is hereby incorporated by reference herein in its entirety for all purposes.

### STATEMENT OF FEDERALLY SPONSORED RESEARCH OR DEVELOPMENT

This invention was made with government support under DMR-1420541 awarded by the National Science Foundation, and under UL1 TR002377 and TL1 TR002380 awarded by the National Center for Advancing Translational Sciences. The government has certain rights in the invention.

### BACKGROUND

Traumatic spinal cord injury (“SCI”) is a devastating condition that results in profound and permanent neurological impairments, including sensory deficits, paralysis, and loss of autonomic function. With a global annual incidence ranging between 250,000 and 500,000, [Ref. 1] SCI continues to be a source of extensive strain on patients and health care systems. At present, there is no treatment available that substantially improves the poor prognosis, multifaceted complications, and significantly diminished quality of life associated with SCI, rendering further research on therapeutic options an imperative.

The central nervous system (“CNS”) has a limited endogenous potential to regenerate and restore function after injury. First, adult CNS axons are restricted in their intrinsic capacity to re-initiate growth after axotomy [Ref 2]. Second, the cascade of events following spinal cord trauma establishes a growth-inhibiting environment at the lesion site, impeding axonal regeneration. The initial mechanical insult leads to immediate disruption of neural tissue with considerable vascular damage and necrotic cell death. Then, secondary degenerative events ensue, which includes ischemic injury, inflammation, reactive gliosis with scar formation, and cystic cavitation—all of which cause progressive tissue loss and neurologic dysfunction [Ref 3].

The complex and multifactorial pathophysiology of SCI illustrates the necessity of combining different therapeutic approaches in order to overcome the physical and molecular barriers to regeneration. Tissue engineering offers the possibility of integrating various strategies. The combination of biomaterials, such as polymer scaffolds, and cell transplantation has received enormous attention in research and represents a highly promising therapeutic approach.

### SUMMARY

Some embodiments of the disclosure provide a matrix for neuron regeneration, the matrix comprising: a sheet having a first surface and a second surface opposite the first surface, the second surface having a plurality of integrally formed ridges; wherein the sheet has a spiral shape, such that the first surface of the sheet faces the second surface of the sheet; and wherein the sheet including the integrally formed ridges, comprises oligo(poly(ethylene glycol) fumarate).

In some embodiments, the first surface includes a patterned surface, the patterned surface including a plurality of channels; and the plurality of channels are substantially parallel.

In some embodiments, each of the plurality of channels have a width and a depth; the width is in a range of 10 μm to 80 μm; and the depth is in a range of 10 nm to 70 nm.

In some embodiments, the first surface includes a metal oxide layer, the metal oxide layer covering at least a portion of the second surface, and the metal oxide layer includes a plurality of channels directed into the metal oxide layer, the plurality of channels being substantially parallel to each other.

In some embodiments, the metal oxide layer comprises a material selected from the group consisting of titanium dioxide, zirconium dioxide, and combinations thereof.

In some embodiments, the matrix further includes a self-assembled alkylphosphonate monolayer disposed on at least a portion of the metal oxide layer.

In some embodiments, the alkylphosphonate monolayer is disposed on the metal oxide layer, and the alkylphosphonate monolayer further defines the plurality of channels, such that a height of each channel is defined by a height of the metal oxide layer and a height of the alkylphosphonate monolayer.

In some embodiments, the metal oxide layer and the alkylphosphonate monolayer defines a patterned surface, and the plurality of channels extend through the entirety of the patterned surface, such that the first surface of the sheet lines a bottom surface of each channel.

In some embodiments, the matrix further includes Schwann cells disposed on the alkylphosphonate monolayer, or within the plurality of channels.

In some embodiments, the matrix further includes human mesenchymal stromal cells disposed on the alkylphosphonate monolayer, or within the plurality of channels.

In some embodiments, the human mesenchymal stromal cells, or the Schwann cells are modified to secrete glial cell-derived neurotrophic factor.

In some embodiments, the plurality of channels are configured to orient a growth of cells along the channel.

In some embodiments, the plurality of ridges are longitudinally spaced apart from each other at a first distance.

In some embodiments, the first distance is in a range of 0.1 mm to 2.0 millimeters.

In some embodiments, the plurality of ridges are laterally spaced apart from each other at a second distance.

Some embodiments of the disclosure provide a matrix for neuron regeneration, the matrix comprising: a sheet having a first surface and a second surface opposite the first surface, the second surface having a plurality of ridges; a metal oxide layer disposed on at least a portion of the first surface; a self-assembled alkylphosphonate monolayer disposed on at least a portion of the metal oxide layer; wherein the sheet has a spiral shape, such that the first surface of the sheet faces the second surface of the sheet; and wherein the sheet comprises a hydrogel.

In some embodiments, the hydrogel comprises oligo(poly(ethylene glycol) fumarate).

In some embodiments, the hydrogel comprises a positively charged oligo(poly(ethylene glycol) fumarate).

In some embodiments, the positively charged oligo(poly(ethylene glycol) fumarate) is a copolymer prepared from oligo(poly(ethylene glycol) fumarate and a cationic monomer.

In some embodiments, the cationic monomer is [2-(methacryloyloxy) ethyl)]-trimethylammonium chloride.

In some embodiments, the plurality of ridges have a height in a range of 25 μm to 150 μm.

In some embodiments, a thickness of the sheet is in a range of 40 μm to 160 μm.

In some embodiments, a total thickness of the sheet, the metal oxide layer, and the self-assembled alkylphosphonate monolayer is in a range of 100 μm to 900 μm.

In some embodiments, the plurality of ridges are longitudinally spaced apart from each other at a first distance.

In some embodiments, the first distance is in a range of 0.1 mm to 2.0 mm.

In some embodiments, the plurality of ridges are laterally spaced apart from each other at a second distance.

Some embodiments of the disclosure provide a method for creating a matrix for neuron regeneration, the method comprising: (a) forming a sheet comprising a hydrogel; and (b) hydrating the sheet such that the sheet forms a spiral shape

In some embodiments, the method further includes forming ridges on a surface of the sheet.

In some embodiments, the method further includes forming a patterned surface on a surface of the sheet, the patterned surface including a plurality of channels, the plurality of channels being substantially parallel.

In some embodiments, the method further includes applying Schwann cells to the patterned surface; and the channels orient growth of the Schwann cells along the channels.

In some embodiments, the method further includes applying human mesenchymal stromal cells to the patterned surface; and the channels orient growth of the human mesenchymal stromal cells along the channels.

In some embodiments, the method further includes forming a metal oxide layer on a portion of a surface of the hydrogel.

In some embodiments, the method further includes curing a metal oxide precursor solution to form the metal oxide layer at a temperature in a range of 10° C. to 50° C.

In some embodiments, the method further includes forming a self-assembled alkylphosphonate monolayer on at least a portion of the metal oxide layer.

In some embodiments, the hydrogel is selected from the group consisting of oligo(poly(ethylene glycol) fumarate), and positively charged oligo(poly(ethylene glycol) fumarate).

Some embodiments of the disclosure provide a matrix for neural regeneration comprising: a sheet having: a first surface having a plurality of channels, the plurality of channels being substantially parallel to each other, and a second surface opposite the first surface and having a plurality of ridges; and wherein the sheet is rolled into a spiral configuration about an axis, such that the plurality of channels extend along the axis, and a portion of the first surface faces a portion of the second surface.

In some embodiments, the channels are defined between adjacent strips of metal oxide layers, the metal oxide layers having a self-assembling monolayer disposed thereon.

In some embodiments, each channel within the plurality of channels has a width, and wherein the width is in a range between 10 μm and 80 μm.

In some embodiments, the channels are directed into the first surface of the sheet, such that the first surface of the sheet defines the channels.

Some embodiments of the disclosure provides a method for creating a matrix for neural regeneration, the method comprising: forming a sheet having a first surface and an opposite second surface; forming ridges on the second surface of the sheet; disposing shadow masks on the first surface of the sheet; and disposing a metal oxide precursor solution on the first surface of the sheet.

In some embodiments, the method further includes curing the shadow masks; curing the metal oxide precursor solution to form a metal oxide layer; and disposing a self-assembling monolayer on the metal oxide layer.

In some embodiments, the method further includes removing the shadow masks to reveal a plurality of channel, the plurality of channels being substantially parallel to each other.

In some embodiments, the method further includes disposing a plurality of cells on the first surface of the sheet, the plurality of cells being at least one of Schwann cells, human mesenchymal cells, and combinations thereof.

In some embodiments, the method further includes disposing neurons on the second surface of the sheet.

In some embodiments, the method further includes hydrating the sheet thereby spontaneously rolling the sheet into spiral configuration.

### BRIEF DESCRIPTION OF THE DRAWINGS

The invention will be better understood, and features, aspects and advantages other than those set forth above will become apparent when consideration is given to the following detailed description thereof. Such detailed description makes reference to the following drawings.

FIG. 1 shows various illustrated views of a matrix for neural regeneration.

FIG. 2 shows illustrated steps of a process used to form a micropatterned chemical on an OPF sheet.

FIG. 3 shows a flowchart of a process for creating a matrix for neural regeneration.

FIG. 4 shows an illustration of a micro-grooved Teflon® mold used to form an oligo(poly(ethylene glycol) (“OPF”) sheet.

FIG. 5A shows a bright field image of a front view of an OPF sheet in a spiral conformation, formed using the mold of FIG. 4.

FIG. 5B shows another bright field image of a side view of the OPF sheet of FIG. 5A.

FIG. 6A shows a light microscopic image of a micropatterned surface of an OPF sheet, taken at 10× magnification.

FIG. 6B shows another light microscopic image of the micropatterned surface of the OPF sheet of FIG. 6A, taken at 2.5× magnification.

FIG. 6C is an image of the template used to form the shadow masks used to create the micropatterned surface of FIGS. 6A, and 6B.

FIG. 7A shows an image of the sheet of FIGS. 6A, and 6B, in a mechanically constrained and relatively flat orientation using pins, prior to cell culturing.

FIG. 7B shows a top view illustration of an OPF sheet being mechanically constrained, similar to the configuration demonstrated in FIG. 7A.

FIG. 7C shows a side view illustration of the OPF sheet of FIG. 7B being mechanically constrained.

FIG. 8A shows a microscopic image taken at 10× magnification, of an OPF sheet having a micropatterned surface formed at a 30° C. heating temperature, and created using a 30 titanium isopropoxide solution diluted with 5 mL of anhydrous toluene (e.g., a 0.02M metal oxide precursor solution).

FIG. 8B shows a microscopic image taken at 10× magnification, of an OPF sheet having a micropatterned surface formed at a 20° C. heating temperature, and created using a 30 titanium isopropoxide solution diluted with 5 mL of anhydrous toluene (e.g., a 0.02M metal oxide precursor solution).

FIG. 8C shows a microscopic image taken at 10× magnification, of an OPF sheet having a micropatterned surface formed at a 40° C. heating temperature, and created using a 30 titanium isopropoxide solution diluted with 5 mL of anhydrous toluene (e.g., a 0.02M metal oxide precursor solution).

FIG. 8D shows a microscopic image taken at 10× magnification, of cultured cells on an OPF sheet having a micropatterned surface created using a 304, titanium isopropoxide solution diluted with 5 mL of anhydrous toluene (e.g., a 0.02M metal oxide precursor solution).

FIG. 8E shows a microscopic image taken at 10× magnification, of cultured cells on an OPF sheet having a micropatterned surface created using a 3 μL titanium isopropoxide solution diluted with 5 mL of anhydrous toluene (e.g., a 0.002M metal oxide precursor solution).

FIG. 8F shows a microscopic image taken at 10× magnification, of cultured cells on an OPF sheet having a micropatterned surface created using a 3 μL titanium isopropoxide solution diluted with 5 mL of anhydrous toluene (e.g., a 0.002M metal oxide precursor solution).

FIG. 9A shows a microscopic image taken at 10× magnification, of cultured cells on an OPF sheet having a chemical pattern comprising a 30 μm width repeating pattern of strips having a titanium dioxide layer and a self-assembled alkylphosphonate monolayer on the titanium dioxide layer, and a 30 μm width channel between respective strips, and a fibronectin layer disposed on the chemical pattern.

FIG. 9B shows a microscopic image taken at 2.5× magnification of cultured cells on the OPF sheet of FIG. 9A.

FIG. 9C shows a microscopic image taken at 10× magnification, of cultured cells on an OPF sheet coated only with fibronectin.

FIG. 9D shows a microscopic image taken at 10× magnification, of cultured cells on an OPF sheet coated with a chemical pattern comprising a titanium dioxide layer, and a self-assembled alkylphosphonate monolayer, having no channels.

FIG. 10 shows a series of microscopic images taken over a period of days, of multiple combinations of cell types cultured on chemically patterned OPF sheets.

FIG. 11 shows a series of immunofluorescent images showing various staining regimens for a cultures of cells on chemically patterned OPF sheets.

FIG. 12 shows immunofluorescent images confirming the presence of human mesenchymal cells, Schwan cells, and dorsal root ganglion neurons in co-cultures.

FIG. 13A shows a graph detailing the equivalent percentages of neurons on the chemical pattern at different growth conditions.

FIG. 13B shows a graph detailing the number of neurons on the chemical pattern at different growth conditions.

FIG. 13C shows a graph detailing the equivalent number of neurons off the chemical pattern at different growth conditions.

FIG. 14 shows a graph of a β-tubulin fluorescent intensity for different growth condition groups.

FIG. 15 shows a series of immunofluorescent images of different staining regimens for various combinations of cell types cultured on chemically patterned OPF sheets.

FIG. 16 shows a graph of a fibronectin fluorescent intensity for different growth condition groups.

FIG. 17 shows a three-dimensional image of the architecture of neurites on a chemically patterned OPF sheet.

FIG. 18A shows an illustration of process flow for creating a chemically patterned OPF sheet.

FIG. 18B shows an illustration of a process flow for culturing cells and measuring neuronal alignment and attachment for a chemically patterned OPF sheet.

FIG. 18C shows an illustration of a process flow for culturing cells and measuring neuronal alignment, attachment, and density for a chemically patterned OPF sheet.

FIG. 19 shows illustrated steps of a process for phosphonate monolayer synthesis on a fully hydrated, hydrophilic, cell-nonadhesive OPF hydrogel. The OPF substrate is treated with a solution of tetra(iso-propyl)titanium as shown by 1 of FIG. 19; hydrolysis of 1 by surface water gives a thin TiO₂ surface layer that reacts with an alkylphosphonic acid to give hydrophobic hydrogel as shown by 2 of FIG. 19, or with a (diphosphono)alkane to give cell-adhesive as shown by 3 in FIG. 19.

FIG. 20 shows graphs of IR spectra for ODPA/TiO₂/OPF at various points. The left hand IR spectra includes a spectra of background-subtracted OPF (upper most line), a spectra before rinsing (lower most line), a spectra after 7 days in PBS (line directly above the lower most line), and a spectra after 30 days in PBS (line directly below the upper most line). The right hand IR spectra was conducted directly after rinsing in ethanol to remove a weakly adsorbed multilayer (day “0” of the 7 day process).

FIG. 21 shows the hydrophobicity of the ODPA/TiO₂/OPF, where the (A) portion shows a droplet bouncing off the horizontal surface and attaches back to the syringe, while (B) shows a droplet rolling off the tilted surface.

FIG. 22 shows various immunostaining images of NIH 3T3 cells plated on unpatterned OPF-SAMP at 40,000 cells/cm². The (A) portion of FIG. 22 was stained for fibronectin and acquired after 1 day, the (B) portion of FIG. 22 was stained for F-actin and acquired after 1 day, the (C) portion of FIG. 22 was stained for fibronectin and acquired after 4 days, and the (D) portion of FIG. 22 was stained for F-actin and acquired after 4 days.

FIG. 23 shows various images of results from the second example. The upper left portion is an image of debossed OPF (grey) with 30 μm×30 μm mask (black) in place (e.g., a repeating pattern of 30 μm width of a mask layer followed by an open channel with a width of 30 [symbol] 0 m and followed by a 30 μm width mask layer), and the (A) and (B) portions are F-actin stained images (with a scale bar of 100 μm) of NIH 3T3 cells plated on debossed 30 μm×30 μm patterned 3 at 40,000 cells/cm² acquired after 1 day (A), and acquired after 4 days (B). The images below (A), and (B) portions are respective FFT images presented in polar form that estimate alignment with these surfaces.

FIG. 24 shows two images, where portion (A) of FIG. 24 shows the rolled OPF+ sheet with ridges spaced 0.4 mm apart and coated with laminin was placed inside a single channel OPF+ ring to hold the rolled sheet together, while portion (B) of FIG. 24 shows the scaffold with a laminin coating integrating with the rat host tissue over a 4 week period.

FIG. 25 shows two images with respective zoomed in portions, where portion (C) of FIG. 25 shows a 10 μm section of the OPF scaffold in the rat spinal cord stained with neurofilament, while portion (D) of FIG. 25 shows a 10 μm section of the OPF scaffold coating with laminin and implanted in a rat spinal cord stained with neurofilament. In both portions of (C) and (D) of FIG. 25, the enwrapping of the scaffold are present and axons are seen passing through.

FIG. 26 shows a graph of the average hindlimb Basso, Beattie, and Bresnahan (“BBB”) motor score for rats implanted with a 7 channel OPF+ scaffolds loaded with matrigel (the circle line), a rolled neutral OPF sheet (square line), a rolled OPF sheet TiSAMP “full” chemical (no pattern, upper pointing triangle line), and a OPF sheet with patterned TiSAMP chemical (30 μm×30 μm, downward porting triangle line). The * indicates a p<0.05.

FIG. 27 shows immunohistochemistry images of 10 μm sections of various sheets. Portion (A) of FIG. 27 shows a 10 μm section of a rolled OPF sheet and the portions (A1, showing the outer edge) and (A2, showing the central region) are zoomed in portions of portion (A). Portion (B) of FIG. 27 shows a 10 μm section of a rolled OPF sheet with full TiSAMP, and portions (B1, showing the outer edge), and (B2, showing the central region) are zoomed in portions of (B). Portion (C) of FIG. 27 shows a 10 μm section of a rolled OPF scaffold with a rolled OPF sheet with a patterned TiSAMP. The staining in FIG. 27 was for B-tubilin, and MBP.

FIG. 28 shows an illustrated steps for a process of creating a matrix for neural regeneration.

FIG. 29 shows various images. Portion (A) of FIG. 29 shows crosslinked OPF+ sheets with 100 μm high ridges that are spaced apart at 0.2 mm, 0.4 mm, and 1 mm respectively. Portion (B) of FIG. 29 shows an OPF+ previously fabricated multichannel scaffold with 7 channels and is as wide as a rat spinal cord. Portion (C) of FIG. 29 shows a graph comparing the swelling ratio of an OPF sheet and the OPF+ sheets of portion (A). Portions (D) and (E) of FIG. 29 show images of the spontaneous conformational change of the OPF sheets. Portion (F) of FIG. 29 shows an illustration of a schematic and mathematical modeling of the surface area of the scaffold ridged sheet. Portion (G) of FIG. 29 shows a graph comparing the surface area of various configurations, where surface area and volume substantially increase for the spiral configuration when comparted to the multichannel configuration of portion (B).

FIG. 30 show various images of DRGs grown on sheets, as described above, coated with laminin in portion (A) of FIG. 30, coated with fibronectin in portion (B) of FIG. 30, coated with collagen in portion (C) of FIG. 30, and coated with serum only in portion (D) of FIG. 30. Portion (E) of FIG. 30 shows a graph of neurite length of the DRG explants on differently coated OPF+ sheets after 24 and 48 hours of culturing. The * compared to 24 hours on serum coated sheets, while # compared to 48 hours on serum coated sheets. The laminin coated sheets displayed significantly longer neurite outgrowth of DRG explants after 24 hours and 48 hours of culture. Portion (F) of FIG. 30 shows a graph of neurite outgrowth rates in the first 24 hours after culture on differently coated 1 mm spaced OPF+ sheets. The neurite outgrowth rate in the first 24 hours of culture of DRG explants on laminin coated sheets were significantly higher than on fibronectin coated and serum coated only sheets. For the graphs of portions (E) and (F), the following symbols mean *p<0.05, **p<0.01, ***p<0.001, ****p<0.0001, and data represent means±SEM.

FIG. 31 shows immunofluorescent images of b-III tubulin stained axons showing attachment and outgrowth from neurons near or on the ridges on OPF+ sheets coated with laminin in portion (A) of FIG. 31, coated with fibronectin in portion (B) of FIG. 31, coated with collagen in portion (C) of FIG. 31, and coated with serum only in portion (D) of FIG. 31. Shown by the white dotted lines, the neurites in portions (A), (B), (C), and (D) of FIG. 31 align with the ridges of the sheet. In all portions of FIG. 31, the neurons were stained with B-III-Tubulin, and a DAPI counter stain.

FIG. 32 shows a series of graphs. Portion (E) of FIG. 32 shows a graph of the number of neurons on ridges (normalized to the number of cells on the ridges with no substrate) of OPF+ sheets coated with laminin, fibronectin, and collage. Portion (F) of FIG. 32 shows a graph of the number of cells in between the ridges (when normalized to OPF+ sheets with serum only). Portion (G) of FIG. 32 shows a graph of the percent of cells on ridges or in between ridges for each condition. Portion (H) of FIG. 32 shows a graph of neurite density as measured by staining intensity (mean gray value) of B-III-Tubilin normalized to the mean gray value of DAPI/cell. For all graphs of FIG. 32, the following symbols mean *p<0.05, **p<0.01, ***p<0.001, ****p<0.0001, and data represent means±SEM.

FIG. 33 shows images and graphs. Portions (A), (B), and (C) of FIG. 33 show immunofluorescent images using B-III tubulin and DAPI for OPF+ sheets with 0.2 mm spaced ridges for portion (A), with 0.4 mm spaced ridges for portion (B), and with 1 mm spaced ridges for portion (C). Portion (D) of FIG. 33 shows a graph of the number of cells on the ridges for the sheets of portions (A), (B), and (C). Portion (E) of FIG. 33 shows a graph of the B-III tubulin staining intensity (normalized to DAPI/cell) of neurons grown on the OPF+ sheets of the portions (A), (B), and (C). For the graphs of FIG. 33, the following symbols mean *p<0.05 compared to the 1 mm spacing as the control, and data represent means±SEM.

FIG. 34 shows various bright field images of rat Schwann cells grown on OPF+ sheets with various ridges. Portions (A), (B), and (C) of FIG. 34 are bright field images acquired after one day of OPF+ sheets having ridges that are spaced apart by 0.2 mm for portion (A), 0.4 mm for portion (B), and 1 mm for portion (C). Portions (D), (E), and (F) of FIG. 34 are bright filed images of the OPF+ sheets of portions (A), (B), and (C) respectively, acquired after 6 days. As shown in portions (D), (E), and (F) of FIG. 34, after 6 days the Schwann cells are aligned along the ridges.

FIG. 35 shows various confocal images showing Schwann cells co-cultured with disassociated DRG neurons on OPF+ sheets with ridges spaced 0.2 mm apart for portions (A), (B), and (C) of FIG. 35, and with ridges spaced 1 mm apart for portions (D), (E), and (F) of FIG. 35. The images of FIG. 35 have been stained with B-III Tubulin (left column), Myelin Basic Protein (middle column), and DAPI (right column). As shown, after culture in media that supports myelination of Schwann cells, the Schwann cells myelinate the disassociated DRG neurites. As shown, there are more aligned myelinated neurites on the 0.2 mm spaced OPF+ sheets.

FIG. 36 shows DAPI staining images of dissociated DRG neurons on OPF+ scaffold sheets with ridges 1 mm spaced apart from each other and coated with laminin in portion (A) of FIG. 36, with fibronectin in portion (B) of FIG. 36, with collagen in portion (C) of FIG. 36, and with serum only in portion (D) of FIG. 36.

FIG. 37 shows various bright field images at 10× magnification of cells grown on OPF sheets with ridges spaced 0.4 mm apart in portions (A), (B), and (C) of FIG. 37, and of cells grown on class coverslips in portions (D), (E), and (F) of FIG. 37. Portions (A) and (D) of FIG. 37 had human MSCs, portions (B) and (E) of FIG. 37 had rat MSCs, and portions (C) and (F) of FIG. 37 had rat Schwann cells.

FIG. 38 shows various bright field images at 10× magnification of human MSCs grown on OPF+ sheets having ridges and having no coating in portion (A) of FIG. 38, having a poly-1-lysine coating in portion (B) of FIG. 38, having a laminin coating in portion (C) of FIG. 38 (taken after 3 days), having a fibronectin coating in portion (D) of FIG. 38 (taken after 3 days), having a laminin coating in portion (E) of FIG. 38 (taken after 7 days), and having a fibronectin coating in portion (F) of FIG. 38 (taken after 7 days). Portion (G) of FIG. 38 is a bright field image at 10× magnification of MSC spheres taken from the experiments of FIG. 38 and cultured on a culture dish.

FIG. 39 shows various images of staining regimens for phallodin, collagen IV, and DAPI of human MSCs cultured on OPF+ sheets with ridges, flat OPF+, and glass. Portions (A), (B), and (C) of FIG. 39 were stained for phallodin, collagen IV, and DAPI, and portions (D), (E), and (F) of FIG. 39 were stained for collagen IV. Portions (A) and (D) of FIG. 39 had OPF+ sheets with ridges, portions (B) and (E) of FIG. 39 had flat OPF+ sheets, and portion (C) and (F) of FIG. 39 had glass.

FIG. 40 shows various images of staining regimens for phallodin, laminin, and DAPI of human MSCs cultured on OPF+ sheets with ridges, flat OPF+, and glass. Portions (A), (B), and (C) of FIG. 40 were stained for phallodin, laminin, and DAPI, and portions (D), (E), and (F) of FIG. 39 were stained for laminin. Portions (A) and (D) of FIG. 40 had OPF+ sheets with ridges, portions (B) and (E) of FIG. 40 had flat OPF+ sheets, and portion (C) and (F) of FIG. 40 had glass.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the use the phraseology and terminology used herein is for the purpose of description and should not be regarded as limiting. Furthermore, the use of “right”, “left”, “front”, “back”, “upper”, “lower”, “above”, “below”, “top”, or “bottom” and variations thereof herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Unless otherwise specified or limited, phrases similar to “at least one of A, B, and C,” “one or more of A, B, and C,” and the like, are meant to indicate A, or B, or C, or any combination of A, B, and/or C, including combinations with multiple or single instances of A, B, and/or C.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. Skilled artisans will recognize the examples provided herein have many useful alternatives and fall within the scope of embodiments of the invention.

As used herein, the term “associated” describes the interaction between two atoms, molecules, biological structures, and combinations thereof. In some embodiments, this interaction can include physical or chemical interactions, or combinations of physical and chemical interactions. Physical interactions can include adsorption, absorption, intermolecular interactions, intramolecular interactions, van der Waals forces, electrostatic interactions, dipolar interactions, dipole-dipole interactions, dipole-induced dipole interactions, charge-dipole interactions, hydrogen bonding, magnetic dipole-dipole interactions, ligand interactions, coordination complexes, entrapment, etc. Chemical interactions can include covalent bonding, ionic bonding, indirect bonding (i.e., an atom or group of atoms that chemically links the two atoms, molecules, or biological structures together), etc.

As used herein the term “bioactive agent” can include any atoms, groups of atoms, molecules, or biological structures (e.g., antibodies) that provide a degree of therapy to a subject. In some cases, the bioactive agent specifically provides therapy to treat a specific disease state of a subject. In some embodiments, the specific disease state can be vascular disorders (e.g., atherosclerosis, aneurysms, arterial/venous stenosis, high blood pressure, arteriovenous malformation, peripheral artery disease, etc.) cardiac disorders (e.g., congestive heart failure, cardiomyopathies, ischemic heart disease, etc.) inflammatory diseases (e.g., chronic kidney disease, Crohn's, inflammatory bowel disease, chronic obstructive pulmonary disease, asthma, rheumatoid arthritis, etc.) regenerative therapies (e.g., heart, liver, pancreas, lung, etc.) metabolic disorders (e.g., obesity, diabetes, dyslipidemia, endocrine, etc.) traumatic disorders (e.g., traumatic brain injury, spinal cord injury, etc.), infections (e.g., fungal, bacterial, orthopedic implant associated, etc.), neurologic disorders (e.g., neurodegenerative diseases, Alzheimer's, Parkinson's, Huntington's, etc.) psychiatric disorders (e.g., attention-deficit/hyperactivity disorder, autism spectrum disorder, depression, etc.), immunology issues (e.g., immunotherapies, etc.), oncology (e.g., glioblastoma, prostate, breast, ovarian, pancreas, head/neck, lung, melanoma, osteosarcoma, etc.), transplants (e.g., liver, kidney, pancreas, heart, etc.), and combinations thereof. In some embodiments, the specific disease state can be chronic, acute, and can be single or multi-symptomatic. In some embodiments, the bioactive agent can include a cytotoxic agent. The cytotoxic agent can include a chemotherapeutic agent, alkylating agents, antagonists, plant alkaloids, intercalating antibiotics, enzyme inhibitors, antimetabolites, mitotic inhibitors, growth factor inhibitors, cell cycle inhibitors, enzymes, biological response modifiers, and combinations thereof. In some embodiments, the bioactive agent can include other pharmaceuticals. In some embodiments, the bioactive agent can include DNA, RNA, or other gene therapeutics.

As used herein the term “generally parallel” or “substantially parallel” implies that two structures or features are close to being parallel, but need not be parallel. For example, two structures can be aligned between 1° and 20° along a parallel shared axis, and any degree in between (e.g., 5°, 7.5°, etc.).

As described above, spinal cord injuries are devastating injuries, with likely life-long impairments. In some conventional approaches, polymer scaffolds have been used to bridge a spinal cord injury site and provide three-dimensional structural support and guidance for regenerating axons [Ref. 4]. Notably, hydrogels composed of oligo[(polyethylene glycol) fumarate] (“OPF”) can be chosen and fabricated, as OPF closely resembles the mechanical properties of the rat spinal cord [Ref. 5], which increases its ability to integrate with the native tissue. The regenerative capabilities of scaffolds can be increased by providing spatial guidance to regenerating axons in order to re-connect spinal cord tracts across the injury. Recently, a nanometer thin, cell-adhesive chemical template has been developed that is able to orient cells along its pattern [Refs. 6, 7].

Combining scaffolds with different cell types can further enhance their regenerative potential by replacing lost cell populations and utilizing supportive properties innate to the cell type. Mesenchymal stem cells (“MSCs”) [Refs. 8, 9] and Schwann cells (“SCs”) [Refs. 10, 11] have consistently been shown to enhance axonal regeneration within the CNS and to improve functional recovery after SCI. SCs are cells native to the peripheral nervous system (“PNS”), which are known to have a greater regenerative capacity. They may exert their effect through demyelinating axons, releasing growth factors, and secreting adhesion and guidance molecules. MSCs are well known for their immunomodulatory and trophic effects, establishing a neuroprotective environment and activating endogenous restorative responses. Additionally, these cells may further enhance regenerative capability by contributing a cell-specific extracellular matrix (“ECM”) vested with native molecules and positional information [Refs. 12, 13]. The MSCs can be easily isolated from bone marrow and adipose tissue.

Previously, hydrogel scaffold implantation complemented with either of MSCs [Ref. 11] or SCs [Ref. 14] have been explored. However, the combination of both cell types in a single scaffold system, has not been utilized. Some main questions left to be answered are: (1) Might axonal regeneration be further increased by synergizing the beneficial properties of both cell types, and (2) how might a hybrid environment that is characterized by different growth factors and signaling molecules innate to the two cell types effect regenerating neurites? Some embodiments of this disclosure, such as detailed in the examples, have provided answers to these questions, and others.

Some embodiments of this disclosure, provide improvements over prior neural generation systems and methods by providing a chemically micropatterned scaffold formed of OPF. The chemically micropatterned scaffold includes a plurality of parallel channels that guide growth of cells placed on the scaffold to extend and grow along the channel. Additionally, the micropatterned scaffold can be co-cultured with SCs and MSCs to produce a hybrid environment for both cell types to grow. The channels and the co-culturing of both cell types can provide better neural generation, and thus can provide a better therapeutic outcome in vivo.

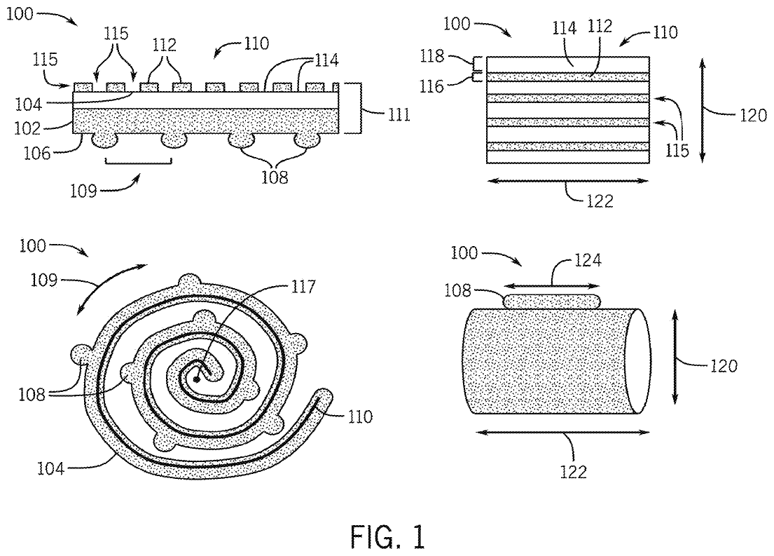

FIG. 1 shows an example of various views of a matrix 100 for neural regeneration, according to some embodiments of the disclosure. Specifically, the upper left quadrant of FIG. 1 illustrates a front side view of a neural matrix 100 in a flat configuration, which includes a sheet 102 having a first surface 104 and a second surface 106 opposite the first surface 104. Protruding from the second surface 106 are a plurality of ridges 108, which are shaped as being spherical, although in some embodiments, the ridges 108 can be shaped other than a sphere (e.g., pyramidal, cubical, other prisms, etc.). For example, in some configurations, the ridges 108 can have a plateau that defines a surface that is substantially parallel to the second surface 106 of the sheet 102. In other words, the ridges 108 can have a substantially flat surface that spans along all (or a portion) of the transverse side 122 (or axial direction) of the matrix 100. In some embodiments, the plurality of ridges can be affixed to the second surface 106, although as illustrated, the plurality of ridges 108 are integrally formed with the sheet 102. Thus, as shown, the ridges 108 and the sheet 102 are formed of the same material. For example, the ridges 108 and the sheet 102 can be formed out of a hydrogel, OPF, OPF+, synthetic polymers, natural polymers, etc. However, in some embodiments, the ridges 108 can be formed from the same or a different material than the material used to form the sheet 102, and affixed to the second surface 106 accordingly. The ridges 108 are spaced apart from each other at a constant longitudinal distance 109, which can be in a range of 0.1 mm to 2.0 mm, or can be specifically, for example, 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, etc. In some preferred embodiments, the constant longitudinal distance 109 can be 1 mm. Disposed on the first surface 104 of the sheet 102 is a micropatterned chemical surface 110. In some embodiments, the sheet 102 can be associated with a bioactive agent (e.g., to stimulate growth of a neuron).

The micropatterned chemical surface 110 can include a metal oxide layer, and a self-assembled monolayer disposed on the metal oxide layer. For example, the metal oxide layer can be disposed on at least a portion of the first surface 104 of the sheet 102. Similarly, the self-assembled monolayer can be disposed on at least a portion of the metal oxide layer. In some embodiments, the self-assembled monolayer is formed from 1,4-butanediphosphonic acid, in other embodiments, the self-assembled monolayer is formed from phosphonate. The upper right quadrant of FIG. 1, illustrates a top view of the neural matrix 100 in the flat configuration, and specifically shows the structure of the chemical patterned surface 110.

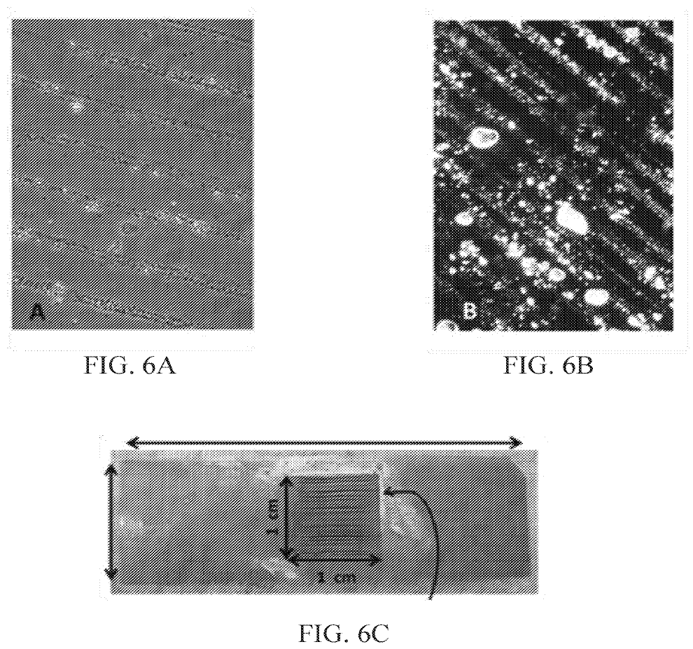

As illustrated, the chemical patterned surface 110 includes a generally repeating pattern of two strips. The first strip 112 has a width 116 and includes a self-assembled monolayer disposed on a metal oxide layer, where the metal oxide layer is disposed on the first surface 104. The second strip 114 has a width 118 and only includes the first surface 104 of the sheet 102 itself, as illustrated in the upper right quadrant of FIG. 1. In other words, and as best illustrated in the following FIGS. (e.g., FIG. 6A), two adjacent first strips 112 define the second strip 114 sandwiched there between, and thus each second strip 114, bounded between adjacent first strips 112 also defines a channel 115. Thus, because the second strips 114 have a height (or thickness) that is smaller than the height (or thickness) of the first strips 112, a channel 115 is defined.

As shown, the channels 115 defined by the second strips 114 are substantially parallel to each other and each have three surfaces: (1) a bottom surface defined by the first surface 104 of the sheet 102, (2) a right side surface generally perpendicular to the first surface 104 and defined by a first strip 112 (e.g., having the metal oxide layer and the self-assembled monolayer) located to the right of the second strip 114, and (3) a left side surface generally perpendicular to the first surface 104 and defined by a first strip 112 (e.g., having the metal oxide layer and the self-assembled monolayer) located to the left of the second strip 114. As illustrated, the channels 115 have an open axial surface, as described below.

In some embodiments, the width 116 of the first strip 112 is identical to the width 118 of the second strip 114, as the pattern repeats along the longitudinal side (or direction) 120 of the neural matrix 100; however, other dimensional relationships are possible. For example, the sizes (e.g., height, width, length) of the first strips 112 can vary, and the sizes (e.g., height, width, length) of the second strips 114 can vary. In specific examples, the widths 116, 118 are in a range between 5 μm to 30 μm, and within the range, such as 5 μm, 10 μm, 20 μm, 30 μm, and combinations thereof. In specific examples, the widths 116, 118 are each 30 μm. In other specific examples, the widths 116 are 30 μm and the widths 118 are greater than 30 μm. The channels 115 are shown as having a square cross-section, although in other configurations, other cross-sectional shapes are contemplated. In some configurations, and as illustrated, the channels 115, which are substantially perpendicular to each other, extend along the transverse side 122 of the neural matrix 100 (e.g., illustrated as extending along the entirety of the transverse side 122). Additionally, when the matrix 100 coils into the spiral configuration, the channels 115 extend along the axial direction of the matrix 100, such that pairs of channels 115 are radially spaced apart from each other, as described in detail below.

In some embodiments, the sheet 102, and the patterned surface 110 can have individual thicknesses and a combined thickness. For example, the sheet 102 can have a thickness of 40 μm, 50 μm, 60 μm, 70 μm, 80 μm, 90 μm, 100 μm, 110 μm, etc. The chemical surface 110 (e.g., the height of the strips 112) can have a in a range of 10 nm to 70 nm, or can specifically be 1 nm, 5 nm, 10 nm, 20 nm, 30 nm, 40 nm, 50 nm, 60 nm, 70 nm, 80 nm, etc. In some embodiments, the sheet 102 and the patterned surface can have a combined thickness 111 in a range of 100 μm to 900 μm, or can be specifically, 100 μm, 200 μm, 300 μm, 400 μm, 500 μm, 600 μm, 700 μm, 800 μm, 900 μm, etc.

The lower left quadrant of FIG. 1, illustrates a front axial view of the neural matrix 100 in a spiral configuration. As illustrated, the second surface 106 having ridges 108, and the first surface 104 having the chemical patterned surface 110, face each other when the neural matrix 100 is in the spiral configuration, but do not face each other when the neural matrix 100 is in the flat configuration. As detailed above, the channels 115 are oriented along an axial axis 117 (e.g., the axial axis 117 being parallel to the transverse side 122) that spans into and out of the page. Due to the spiral configuration of the matrix 100, some of the channels 115 are positioned at a radially distance to other channels 115 relative to the axial axis 117. This way, cells can be disposed and can extend along respective channels 115 at many different radial locations of the matrix 100 in the spiral configuration. In other words, a cross-section of the matrix 100 in the spiral configuration (e.g., similarly to the view in the bottom left quadrant of FIG. 1A) can have a substantially high number of cell guiding sties (e.g., channels 115) relative to the cross-sectional area of the matrix 100.

The lower left quadrant of FIG. 1 also shows some ridges 108 being radially displaced from other ridges 108 relative to the axial axis 117. In some embodiments, and as illustrated for some of the ridges 108, ridges 108 on a given loop of the swirl can be staggered relative a given ridge 108 on an adjacent loop concentric to the given loop of the swirl. In other words, a ridge 108 on one loop of the swirl can be positioned between adjacent ridges on another loop of the swirl adjacent to and concentric with the one loop of the swirl. Stated another way, a ridge 108 on one loop can be concentrically positioned between two ridges 108 on an adjacent loop. In some embodiments, and as also illustrated in the lower left quadrant of FIG. 1, a given ridge 108 of a loop of the swirl can be concentrically aligned with another ridge 108 on another loop of the swirl adjacent to the loop of the swirl (e.g., the two ridges 108 located in the lower left quadrant of FIG. 1).

As will be described in further detail below, when the neural matrix 100 becomes hydrated (e.g., absorbs water), the neural matrix 100 spontaneously changes its shape from the flat configuration to the spiral configuration (e.g., changing in shape from the view in the upper left quadrant of FIG. 1 to the view of the lower left quadrant of FIG. 1).

The lower right quadrant of FIG. 1, illustrates a front side view of the neural matrix 100 in a spiral configuration. As shown, each ridge 108 extends along the transverse side 122 (or axial direction) of the matrix 100 by an axial distance 124, which can be a percentage of a length of the transverse side 122 (e.g., 50%, 80%, 90%, etc.). In some embodiments, the axial distance is greater than 100-180 μm (e.g., the diameter or thickness of the ridges, dry or after hydration), greater than 2 mm, greater than 3 mm, etc. In some embodiments, each ridge 108 extends along the entire transverse side 122 of the matrix 100. In some embodiments, each ridge 108 can be segmented along the transverse side 122, such that portions (e.g., two, three, etc.) of each ridge 108 extend along the transverse side 122 by a specific amount, where each portion is separated by a gap. For example, at least one ridge 108 can have multiple partitions other than the illustrated one, such as two, three, four, five, etc., that span along the transverse side 122 (or along the axial axis 117).

Although FIG. 1 has been illustrated and described as having the chemical pattern 110 that defines the channels 115 (e.g., between adjacent first strips 112), in other configurations, the channels 115 can be formed in the first surface 104 of the sheet 102, so as following the same shape as described above (e.g., the channel 115 being open to the ambient environment along the transverse side 122). For example, the sheet 102 can be molded, manufactured (e.g., by cutting away material) to form the channels 115 having various widths (e.g., the width of the strip 112), depths (e.g., directed into the first surface 104 of the sheet 102), and lengths (e.g., along the transverse side 122, such as along the entirety of the transverse side 122), and as being open (lacking material) along the transverse side 122. This way, the channels 115 can have a depth that is entirely defined by the sheet 102. So, in essence, the channels 115 can have the strips 112 be the same material as the sheet 102. Additionally, the channels 115 can also be further defined by the chemical pattern 110 deposited on the first surface 104 of the sheet 102, as described above, such that the strips 112 being the same material as the sheet 102 have the metal oxide layer deposited on these strips 112, and the self-assembled monolayer being deposited on the metal oxide layer. In this configuration, the channels 115 can have a depth that is defined by the depth of the metal oxide layer, the depth of the self-assembled monolayer, and the depth of the strips 112 being the same material as the sheet 102.

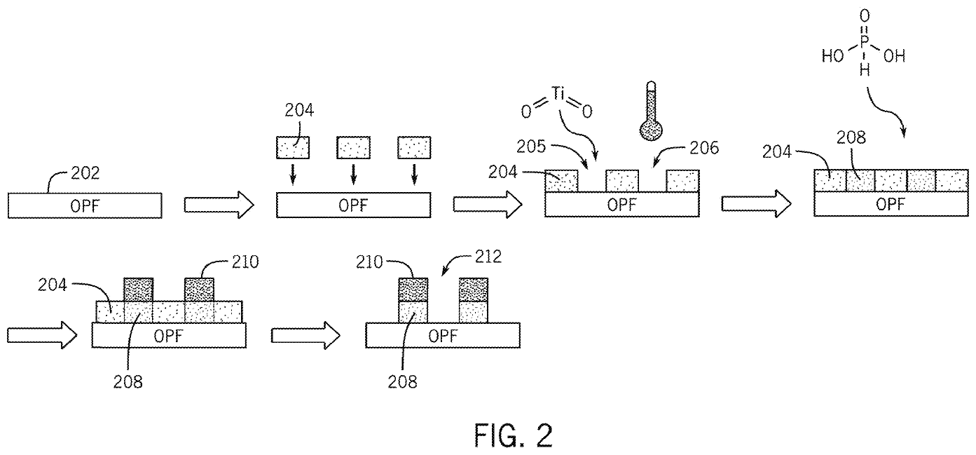

FIG. 2 shows illustrated steps of a process used to form a micropatterned chemical surface (e.g., the micropatterned chemical surface 110) on an OPF sheet 202. For example, the process starts with depositing shadow masks 204 (e.g., created with a laser writer) on a surface the OPF sheet 202, which forms channels 205 between adjacent deposits (or strips) of the shadow mask 204 layer. Then, a solution of a metal oxide precursor 206, for example to eventually generate TiO₂, is placed on the OPF sheet 202 that includes the shadow masks 204, such that the solution of the metal oxide precursor 206 is received in the channels 205. Once sufficiently coated, the OPF sheet 202 having the shadow masks 204, and the solution of the metal oxide precursor 206 is cured accordingly with, for example, increased temperatures. In some embodiments, curing at a particular temperature forms the metal oxide and effectively cross links the metal oxide (e.g., TiO₂) that is in the solution of the metal oxide precursor 206 to the OPF surface. Additionally, the water from the sheet itself can cure and create the metal oxide (e.g., by hydrolysis). After sufficient curing, a metal oxide layer 208 forms between adjacent strips of the shadow mask 204. In other words, the metal oxide layer 208 forms in the location of the metal oxide precursor solution 206, or stated another way, where the channels 205 had been located (e.g., the metal oxide precursor solution 206 being disposed in the channels 205). Then, a phosphonic acid solution is loaded on the OPF sheet 202, forming a self-assembled monolayer 210 that is disposed on the metal oxide layer 206 (and which portions may form on the shadow mask layer 204). Then, the shadow masks 204 are removed from the OPF sheet 202 (e.g., by manual removal, such as with a forceps, or with washing with a solution, such as toluene, isopropanol, or both, a number of times such as three times), ultimately revealing a plurality of channels 212 (e.g., as described above with regard to neural matrix 100). Although this process was used in the example below to generate a 30 μm×30 μm TiO₂/SAMP pattern (e.g., a 30 μm width repeating pattern of the TiO₂/SAMP layer followed by a 30 μm width channel), other dimensions are possible. For example a 20 μm×20 μm, a 40 μm×40 μm, a 50 μm×50 μm, a 60 μm×60 μm TiO₂/SAMP pattern is possible.

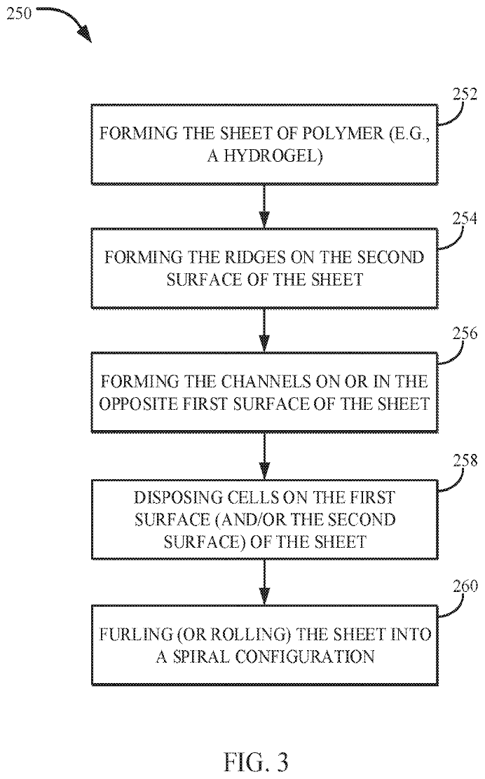

FIG. 3 shows a flowchart of a process 250 for creating a matrix (e.g., the matrix 100) for neural regeneration. At 252, process 250 includes forming the sheet (e.g., the sheet 102) of a polymer, such as a hydrogel (e.g., OPF). In some cases, a mold (e.g., the mold 280) is utilized to form features of the sheet, such as the ridges (e.g., the ridges 108). For example, a polymer solution can be poured onto the mold, such that the solution fills into cavities (or other features in the mold) to create the ridges, or other features of the sheet, when the polymer solution is cured (e.g., by UV light) to form the polymer.

At 254, process 250 includes forming the ridges on the second surface of the sheet. As described above, the sheet can be produced from a mold, such that the second surface of the sheet has the plurality of ridges (e.g., integrally formed ridges), which can be spaced apart from each other at specific distances (e.g., 1 mm in the longitudinal direction), and which can be formed from cavities in the mold. In other configurations, the ridges can be coupled to the second surface of a sheet (e.g., with an adhesive), or with a layer of uncured polymer (e.g., the solution of OPF). This way, by curing the uncured polymer, the ridges become bonded to the second surface of the sheet. The ridges can embody different shapes, such as being hemispheres, spherical, round, etc.

At 256, process 250 includes forming the channels on the opposite first surface of the sheet. In some cases, the mold can be configured to create a plurality of channels on the opposite first surface of the sheet (e.g., the surface opposite to the plurality of ridges). This way, the material of the sheet can at least partially define the channels, as described above. For example, the plurality of channels can be directed into the first surface of the sheet, such that the first surface of the sheet defines the channels (e.g., the channels being defined by at least the material of the sheet). As described previously (e.g., FIG. 1), the channels can be formed so that they are substantially parallel to each other, have a specific depth, have a specific width (e.g., 30 μm), are separated from adjacent channels by a width (e.g., 30 μm), extend along a transverse side (e.g., extend axially when the sheet is rolled up), and have an open longitudinal surface.

Additionally or alternatively, to the channels being formed directly into the first surface of the sheet, the channels can also be supplemented with a chemical pattern, or the channels can be formed independently on the first surface of the sheet with the chemical pattern. The channels can be formed independently or can be further defined with the process described above with regard to FIG. 2. For example, first, the provided sheet (e.g., including the ridges on the second surface) can be unfurled, or unrolled, so as to expose the first surface of the sheet. Then, the sheet can be mechanically fixed, with pins, weights (e.g., 200 grams), etc., to ensure that the sheet does not furl (or roll back up). A shadow mask can then be applied in a pattern on the first surface of the sheet, that will eventually form the channels. In the case of the channels being directly formed into the first surface of the sheet, the shadow masks can be disposed in the channels, and built up (or layered) to a desired height. The shadow masks (e.g., the template having the channels) can be disposed on the first surface. For example, the shadow masks (or the template) include substantially parallel strips of a width and can be placed on the first surface of the sheet. If needed, the shadow masks (or the template) can be aligned, such that the parallel strips are aligned with the channels (if formed previously).

After the shadow masks have been disposed on the first surface of the sheet, the sheet with the template, shadow masks, and mechanically fixed components (e.g., 200 gram weights) can be heated to a temperature (e.g., 30° C.) for a time (e.g., 15 seconds) to cure or otherwise solidify the shadow masks. After the shadow masks are cured, the metal oxide precursor solution (e.g., titanium dioxide precursor solution) can be applied to the first surface of the sheet. In some embodiments, the metal oxide precursor solution can be an organic oxide compound, such as, titanium (IV) isopropoxide dispersed in an hydrophobic solvent (e.g., anhydrous toluene). In some embodiments, the metal oxide precursor solution has a molarity in a range between 0.05M and 0.04M, in some cases the molarity of the metal oxide precursor solution is greater than or equal to 0.02M, in some cases, the molarity of the metal oxide precursor solution is 0.02M, in other cases, the molarity of the metal oxide precursor solution is greater than 0.002M. In some embodiments, the metal oxide precursor solution can be applied to the first surface of the sheet (with the shadow masks) by submersing the first surface of the sheet in the metal oxide precursor solution, and followed by a wash with anhydrous toluene. This submersing and washing can be repeated a number of times (e.g., three times). Following the application of the metal oxide precursor solution, the sheet with the shadow masks, and metal oxide precursor solution, can be cured at a temperature (e.g., 30° C.) for a time period (e.g., 30 seconds) to create a metal oxide (e.g., titanium dioxide) that is cross-linked to the first surface of the sheet (e.g., the polymer, such as OPF). After curing at the temperature, the sheet can be cooled (at a temperature lower than the temperature, such as ambient temperature). Then, a self-assembling monolayer solution can be applied to the first surface of the strip. In some embodiments, the first surface of the strip having the metal oxide layer can be submersed in the self-assembling monolayer solution for a period of time (e.g., 12 hours) to create the self-assembling monolayer. After creating the self-assembled monolayer on the metal oxide layer, the shadow masks can be removed to reveal the channels. In some cases, the chemical pattern (e.g., the metal oxide layer, and the self-assembling monolayer) entirely defines the channels, however in other cases, the chemical pattern along with the preformed channels directed into the first surface of the sheet all define the channels.

At 258, process 250 includes disposing cells (and other components) on the first surface of the sheet, the second surface of the sheet, or both surfaces of the sheet. Prior to disposing cells on the surface(s) of the sheet, the surface(s) of the sheet can be loaded with a layer of fibronectin, and other ECM molecules (e.g., laminin, collagen, etc.). In some embodiments, a co-culture of cells can be disposed on a given surface of the sheet. For example, Schwan cells and human mesenchymal cells can be deposed on the given surface of the sheet. As another example, either Schwan cells or human mesenchymal cells can be disposed on the given surface of the sheet. Additionally, neurons (e.g., from dorsal root ganglia) can be disposed on the co-cultured sheet, or independently cultured sheet (e.g., Schwan or human mesenchymal cells alone). After the desired cells are disposed on the desired surface(s) of the sheet, the sheet having the cells can be cultured (e.g., at 37° C.) for a period of time to allow attachment and growth of the cells along the channel.

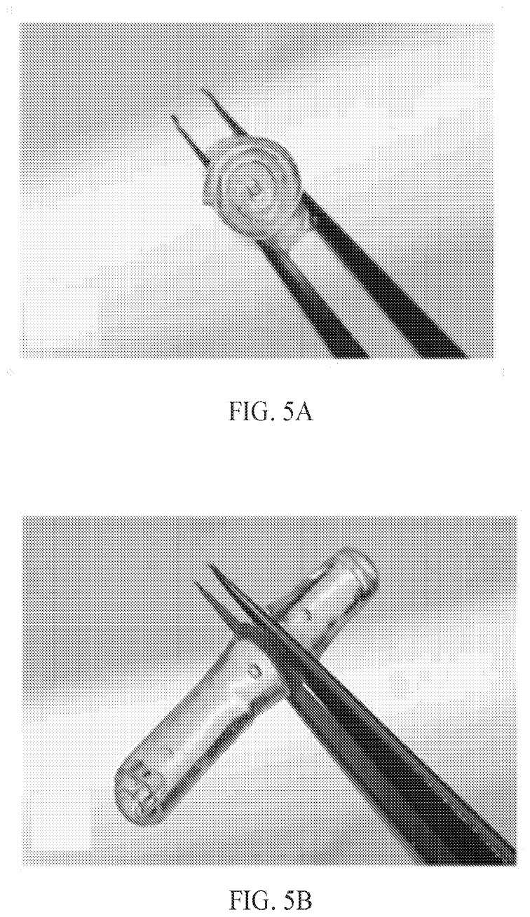

At 260, process 250 includes furling or rolling up the sheet into a spiral configuration (e.g., FIGS. 1, 5A, and 5B). In some embodiments, the sheet spontaneously furls up into the spiral configuration when the sheet is hydrated with a solvent (e.g., a hydrophilic solvent, such as water). In some cases, hydrating the sheet to roll (or furl) up the sheet includes applying droplets of a solvent (e.g., water) to the sheet until the sheet furls up. The spiral configuration allows the channels to be oriented axially (e.g., axially relative to the shape of the spiral). This way, the substantially parallel channels are orientated axially, and at various radial positions throughout the spiral. This can be advantageous in that there is a large ratio of channels relative to an cross-sectional area of the matrix (e.g., the view of the lower left quadrant of FIG. 1). In other words, there are a large number of cell elongation sites (e.g., the cells elongating along the channels) for the cross-sectional area of the spiral.

### EXAMPLES

The following examples have been presented in order to further illustrate aspects of the disclosure, and are not meant to limit the scope of the disclosure in any way.

### Example 1

Neuroregeneration after spinal cord injury is limited and unfortunately, to date, there is no therapy available that reverses the neurological impairments caused by trauma to the cord. Combinatorial treatments need to be investigated, since it may take multiple routes to promote a growth-enhancing environment and axonal regeneration. There is significant scientific precedent for the use of biomaterial scaffolds combined with cell transplantation; additionally, self-assembled extracellular matrix (“ECM”) scaffolds are an emerging field of research. We therefore aimed to generate a three-dimensional, cell-derived hybrid matrix by co-culturing human mesenchymal stem cells (“hMSCs”) and Schwann cells (“SCs”) onto oligo[(polyethylene glycol) fumarate] (“OPF”) scaffold sheets. The scaffolds were micropatterned with a nanometer thin chemical interface for ultrastructural cellular alignment. Without being bound by theory, we hypothesized that a co-culture of hMSCs and SCs on chemically patterned OPF scaffolds enhances neurite alignment, attachment and density.

In some embodiments, OPF scaffolds were chemically micropatterned using a titanium dioxide precursor solution and phosphonic acid to obtain 30 μm by 30 μm stripes (e.g., 30 μm width stripes separated by 30 μm width channels between adjacent titanium dioxide and phosphonic acid stripes) across the hydrogel surface. hMSCs and SCs were co-cultured in a 50:50 ratio onto the OPF sheets for 10 days. On the last 4 days, dissociated neurons from rat dorsal root ganglia (“DRG”) were cultured either onto the cell monolayer or onto fibronectin coated scaffolds. Unpatterned and single cell type-scaffolds were used as controls. Immunofluorescent images were captured to determine neurite alignment and density (via 0-tubulin fluorescent staining intensity) as well as to quantify neuronal attachment on the scaffolds. The amount of cell-deposited fibronectin was quantitated by measuring fibronectin fluorescent intensities to determine this component of the ECM.

The chemically patterned OPF scaffolds promoted a high degree of neuronal attachment on the chemical and alignment along the bidirectional stripes irrespective of cell type. Seeding scaffolds with co-culture of hMSCs and SCs or SCs alone significantly enhanced neuronal attachment and extension when compared to fibronectin coated scaffolds. In addition, co-cultures led to a significantly higher neurite density in comparison to hMSCs. Notably, co-cultures with dissociated neurons supported a significantly greater fibronectin deposition than each cell type alone with neurons.

In some embodiments, and without being bound by theory, the present study highlights the ability of a SC and hMSC co-culture in augmenting the neuro-regenerative potential of both cell types by synergizing their beneficial properties and enhancing their ECM deposition. The study further underlined that the two-component chemical micropatterns provide cell-attractive, micro-topographical cues to spatially direct neurite outgrowth and ECM fibril orientation on the surface of OPF scaffolds.

### Literature Review

### Spinal Cord Injury in General and its Epidemiology

Traumatic SCI is defined by any physical impact inflicting damage on spinal cord tissue and constitutes up to 90% of SCI cases [Ref. 1]. Motor vehicle accidents represent the leading cause (38.4%), followed by falls (30.5%), acts of violence (13.5%), and sport accidents (8.9%) [Ref 15]. Albeit a minority, nontraumatic SCIs are anticipated to increase and are most frequently a consequence of cancer or infection [Ref 16]. However, traumatic causes remain the pivotal focus of SCI research, including the one conducted in our laboratory.

Globally, the reported annual incidence of SCI ranges between 250,000 and 500,000, [Ref. 1] equating to approximately 23 cases per one million of the population [Ref. 17]. With an incidence of 17,500 new cases per year, [Ref 15] SCI occurs more frequently in the United States than in other countries, presumably due to higher rates of road accidents and violence [Ref. 18]. SCI predominantly affects people aged 15-29 years and those above 65 years of age, resulting in a bimodal age distribution [Ref 19]. Due to the population's rising life expectancy, the SCI landscape is gradually shifting towards a higher median age of injury: from 28.3 years in 1970 to 42 years at present [Ref. 15]. Correspondingly falls, a major cause of SCI among the elderly, are continually increasing in number [Ref 18]. In terms of gender, SCI is more prevalent in the male population, with a male-to-female ratio of at least 3:1 [Refs. 15, 18]. The proportion of females is, however, expected to increase—a foresight attributed to the aging of the population.

Clinically, SCIs can be fundamentally classified as being neurologically complete or incomplete. In complete injuries both sensory and motor function are absent below the level of injury, while some preservation of sensory and/or motor function can be found in incomplete injuries [Refs. 20, 21]. Owing to modern medical advances, complete SCIs have decreased overtime (from 53.6% in 1970 to 48.7% since 2000) and incomplete injuries are seen more commonly [Ref. 22]. Contusion/compression of the spinal cord represents the majority of injuries (¾), while open cord lacerations concomitant with complete or incomplete transection lesions are less prevalent (¼) [Ref. 23]. The cervical spine represents the anatomical area most frequently affected, accounting for 55.7% of SCIs, followed by the thoracolumbar junction [Ref. 22]. While respiratory compromise is very likely to occur at any injury level, impairment in the cervical spine is most detrimental to respiratory function by inflicting damage on phrenic nerve motor nuclei [Ref 23]. Overall, respiratory complications embody the leading cause of morbidity and mortality in both early and late SCI stages [Ref 24]. Beyond that, septicemia, bladder and bowel dysfunction, autonomic dysreflexia, spasticity, chronic pain syndromes, and pressure ulcers constitute major long-term, secondary complications of SCI [Refs. 1, 24].

Advancements in SCI research and medical management have led to greater post-injury survival over the past decades. However, clinically approved treatments leading to regeneration, neurologic and functional recovery are lacking and effective treatments that target the multiple pathophysiological mechanisms of SCI remain to be elucidated.

### SCI Pathophysiology and Impediments to Endogenous Regeneration

The spinal cord has limited endogenous potential to regenerate and re-establish neuronal pathways and function after injury. In contrast to the PNS, axons in the fully developed mammalian CNS are restricted in their intrinsic capacity to re-initiate growth after axotomy and Wallerian degeneration [Ref 2]. Given the right environment, research has established that neurons do present some regenerative ability, which is dependent on neuronal type and age, [Ref. 25] and can be enhanced by providing neurotrophic factors [Ref. 26]. Even so, however, CNS neurons do not succeed in substantial, targeted re-growth, resulting in poor functional recovery. [Ref 25, 27]. In addition, the cascade of events following spinal cord trauma creates a highly growth-impeding microenvironment, which compounds on the poor intrinsic capacity of neural cells to regenerate.

SCI results in a complex, biphasic pathophysiologic process, commencing with the primary phase which is the initial mechanical insult to the cord. Compression or traction forces lead to immediate disruption of neural tissue with considerable vascular damage and necrotic death of neuronal and glial cells, causing neurological deficits below the level of injury [Ref 3]. Edema and microhemorrhages within the grey matter ensue, result in secondary ischemia as venous blood pressure is increased by cord swelling [Ref. 28]. The primary injury induces the succeeding secondary phase—a propagating wave of cellular, vascular and biochemical events, which compounds the initial trauma by inflicting damage on any residual neurons and glia. As data increasingly suggest, it is in fact the protracted secondary injury that hinders neuroregeneration [Ref. 29].

A key contributor to necrotic and apoptotic cell death immediately after SCI is excitotoxicity concomitant with loss of ionic homeostasis. Pathologically increased levels of extracellular glutamate and consequent overactivation of α-amino-3-hydroxy-5-methyl-4-isoxazolepropionic acid (“AMPA”) and N-methyl-D-aspartate (“NMDA”) receptors lead to excessive calcium influx [Ref. 30]. This leads to mitochondrial dysfunction with release of reactive oxygen species, activation of degrading enzymes, and inadequate ATP production [Ref, 31, 32]. Free radical formation also emerges as a consequence of ischemia, reperfusion, and immune infiltration, inflicting oxidative stress and consequently self-perpetuating lipid peroxidation and cell decay [Refs. 33-35]. Furthermore, apoptosis in oligodendrocytes and neurons was demonstrated to be in part triggered by stimulation of the Fas receptor and via p75 receptor signaling, compounding myelin and neuronal loss [Refs. 36, 37].

Neuroinflammation after SCI is highly complex and controversial, with some aspects contributing beneficially to the lesion microenvironment (e.g., clearance of growth-impeding CNS debris), and others inflicting neurotoxic, secondary injury. Microglia are one of the first CNS cells reacting to trauma. They transform into an active state with amoeboid morphology, aiming to enclose the injury site, and upregulate inflammatory cytokines, including TNF-α and IL-1β [Refs. 38, 39]. After 3 days, macrophages become activated followed by a wave of infiltrating lymphocytes [Ref. 40]. Inflammation, in conjunction with direct vascular trauma, figure prominently in increasing blood-spinal cord barrier (“BSCB”) permeability and hemorrhaging [Ref 41]. This results in infiltration of non-CNS molecules, such as fibrinogen, and in the induction of the transforming growth factor beta (TGF-β)/Smad signaling pathway leading to the activation of astrocytes [Ref. 42].

Predominantly at the periphery of the lesion, astrocytes proliferate and become hypertrophic—a process referred to as reactive gliosis. This process is characterized by the upregulation of intermediate filaments such as glial fibrillary acidic protein (“GFAP”) [Ref. 43]. Notably, reactive astrocytes abundantly express inhibitory extracellular matrix components of the chondroitin sulfate proteoglycan (“CSPG”) family, comprising aggrecan, neurocan, brevican, versican, phosphacan, and neuron-glial antigen 2 (“NG2”) [Ref. 44]. Their axonal growth-inhibiting nature is well established, [Refs. 45, 46] which is mediated by activation of the receptors leukocyte common antigen-related phosphatase (“LAR”) and protein tyrosine phosphatase sigma (“PTPσ”). [Ref. 47] Subsequent downstream signaling via the Rho/ROCK pathway, amongst others, has been implicated in growth cone collapse [Ref. 47-49].

Concurrently, activated astrocytes extend large, intertwining cytoplasmic processes, which border the lesion to contain inflammation and demarcate surrounding healthy tissue [Refs. 50, 51]. Overtime a glial scar forms beyond which regenerating axons cannot extend [Refs. 51, 52]. The glial scar thus represents a major impediment to regeneration by embodying a physical as well as a molecular barrier. Additively, a fibrotic scarring response hampers axonal regeneration attempts. The release of TGF-β2 by macrophages, [Ref 53] amongst others, induces perivascular fibroblasts [Ref 54] and pericytes [Ref. 55]. These cells subsequently migrate towards the core of the lesion and deposit extracellular collagen—yet another physical obstacle to elongating axons [Ref. 56].

Notably, CNS myelin can also inhibit regeneration. Following injury, it accumulates as debris and releases myelin associated inhibitors (“MAIs”), including Nogo-A, [Ref. 57] myelin-associated glycoprotein (“MAG”), [Ref 58] oligodendrocyte-myelin glycoprotein (“OMgp”), [Ref. 59] and semaphorin 4D [Ref 60]. They all induce axonal growth cone collapse via activation of Nogo receptor 1 and its co-receptors (e.g. TROY, [Ref 61] p 75, [Ref 62] LINGO-1 [Ref 63]), activating Rho/ROCK downstream signaling [Ref 64].

These diverse and numerous barriers in concert with the limited intrinsic restorative potential prevent axons from regenerating beyond the scar tissue and re-integrating neuronal pathways. The chronic phase of the secondary injury (1-2 years post-SCI) is hallmarked by maturation of the glial scar, cystic cavitation, and sometimes formation of syrinxes [Ref. 65]. The challenge in research is to surmount these barriers to neuroregeneration and bridge the gap of non-functional tissue in order to achieve neurologic recovery.

### Current Treatment

At present, there is no effective treatment available that reverses the neurological deficits resulting from SCI. Clinical interventions for acute spinal cord injury comprise high-dose methylprednisolone sodium succinate (“MPSS”), hemodynamic control, and surgical decompression followed by stabilization. The former is currently the only pharmacologic therapy shown to improve functional recovery, when administered within eight hours of injury for one or two days, as indicated by a systematic review in 2012 [Ref. 66]. However, its use after SCI remains a topic of controversy, partially due to safety concerns [Refs. 67, 68]. The same pertains to surgical interventions, with an ongoing debate regarding optimal timing [Refs. 69-71]. In chronic SCI therapeutic options are limited to rehabilitative care and physiotherapy. While progressive insight into the cellular and molecular events post-injury and modern medical advances have improved patient outcome, there is still an unmet need for a therapeutic standard of care. These include neuroprotective treatments, which preserve cells by mitigating secondary injury, as well as neuroregenerative approaches, aiming at recovering severed axonal tracts and therefore chronic deficits. The intricate, multifactorial, and multiphasic nature of SCI necessitates a combinatorial therapeutic approach.

### Tissue Engineering

As aforementioned, glial scarring and cystic cavitation post-injury embody barriers to regeneration. There are two possibilities to bridge this gap and consequently re-establish neuronal pathways. One approach is to bypass the block by using nerve autografts, which re-connect regions above injury level to peripheral targets, such as motor end plates or nerve roots [Ref. 72, 73]. The second option is to replace the scar and cyst areas with bioengineered tissue and grafts, thereby creating a structural guide for regenerating axons.

Tissue engineering constitutes a highly promising approach to SCI treatment, providing a wide array of options to modulate the inhibitory environment at the lesion site into a growth-permissive one to enhance regenerative processes. In the past years, biomaterial polymer scaffolds, in particular, have aroused attention due to their unique biological and mechanical properties, and the versatility with which their chemistry and architecture can be adjusted. The latter enables them to be applied in diverse ways: as injectable gels, suitable for contusion and small tears, or as tubes and multichannel scaffolds designed to bridge larger gaps as found in transection injuries [Ref 23]. The scaffolds can be further converted into delivery systems via incorporation of therapeutic drugs, [Ref 74] cells, and growth factors [Refs. 75, 76].

Although the complete transection lesion accounts for a minority of patients, it is predominantly used as a lesion model in animal research. For one thing, as no axonal tracts are spared, truly regenerating axons can be studied and analyzed in a well-controlled manner [Ref. 77]. Additionally, transection lesions repaired by scaffold engraftment provide an environment, which can be precisely directed and manipulated in order to augment neuroregeneration.

In this regard, the concept of placing three-dimensional polymer scaffolds into spinal cord transection models has attracted immense attention in research and has achieved promising preclinical results in terms of axonal regeneration and locomotor improvement, particularly in combination with cell therapy [Refs. 4, 11, 14, 78].

### Synthetic Polymer Scaffolds

The scaffold's architecture, intrinsic chemical and physical properties of the employed polymer and additional surface modifications influence cell-matrix interaction affecting the graft's potential to regenerate axons. Importantly, polymer scaffolds must be biocompatible within the spinal cord environment to promote graft integration and survival. Biocompatibility encompasses properties such as permeability, stiffness, strength, degradation kinetics and products, swelling, and porosity [Ref. 23]. The latter has been shown to allow for graft vascularization and facilitate cell attachment, migration and differentiation [Refs. 79, 80]. With an overall advantageous effect on neuronal regeneration, [Ref. 80] porosity is thus an important attribute to be considered. In this respect, hydrogels have been highly investigated in biomaterial research and consistently been demonstrated to be an effective medium for SCI repair [Refs. 81-83]. They are macroporous, highly aqueous polymeric networks, readily enabling nutrient diffusion and cell migration. These soft materials resemble spinal cord tissue in their mechanical properties, [Ref. 23] improving graft integration, and function as sophisticated drug—, [Refs. 74, 84] gene—, [Ref. 85] and cell—[Ref. 86] delivery vehicles.

Biomaterial polymers can also be classified as naturally derived, synthetic biodegradable or synthetic non-biodegradable. Type I collagen, [Ref. 87] agarose, [Ref. 82] hyaluronic acid, [Ref. 88] chitosan, [Ref 89] fibronectin and fibrin [Ref 90] constitute the main natural hydrogels that have been explored in SCI models so far. As data suggest, all of them exert beneficial effects on axonal preservation and regeneration mainly through their inherent bioactive properties and functions, with the advantage of being naturally remodeled and having low antigenicity [Ref. 23]. Synthetic polymers studied as yet in the spinal cord comprise biodegradable materials based on polyesters of lactic and glycolic acid (“PLA” and “PGA” respectively), biodegradable hydrogels based on polyethylene glycol (“PEG”), and non-biodegradable hydrogels based on methacrylate [Ref 23]. A core advantage of synthetic polymers is that their utilization enables greater flexibility and scope in terms of material design and manipulation of chemical and physical properties, by adjusting polymer concentration or concentration ratios [Ref. 91]. This allows for fine tuning according to the desired application form and optimization of key polymer parameters.

Oligo[(polyethylene glycol) fumarate] (“OPF”), in particular, is an attractive synthetic hydrogel for use in spinal cord bioengineering applications due to its high compatibility with native cord tissue. OPF is a polyethylene glycol (“PEG”) based macromer with an incorporated fumarate moiety. It is photo-cross-linked with a thermal radical initiation system to become a soft, biocompatible hydrogel, having properties which can be easily be adjusted by changing cross-linker/polymer ratio [Ref 92]. Further, modulation can be achieved via OPF copolymerization with [2-(methacryloyloxy) ethyl)]-trimethylammonium chloride (“MAETAC”), resulting in a positively charged hydrogel (“OPF+”) [Ref 93]. In a quantitative comparison of four different synthetic, multi-channel scaffolds, namely poly(lactic co-glycolic acid) (“PLGA”), poly(ε-caprolactone fumarate) (“PCLF”), OPF and OPF+ hydrogel, for implantation with Schwann cells into transection animal models, OPF and OPF+ hydrogels were demonstrated to most closely resemble rat spinal cord in terms of their mechanical properties [Ref. 5]. While all polymers promoted axonal regeneration, it was found that OPF+ significantly led to the highest number of axons with a more centralized distribution. In complete spinal cord transection animal models, OPF+ scaffolds seeded with Schwann cells were demonstrated to not only augment axonal regeneration, [Ref 5] but also support a growth-permissive milieu at the lesion site by attenuating secondary injury [Ref. 94]. Despite these promising effects, there was no significant improvement of hindlimb function noted [Ref 5]. However, recently, functional improvement could be achieved by implanting OPF+ scaffolds loaded with genetically modified SCs that secrete high concentrations of glial cell-derived neurotrophic factor (“GDNF”) [Ref 14]. This demonstrates the value of investigating different combinatorial treatments for SCI, since it may take multiple routes to elicit neuroanatomical and functional recovery.

### Scaffold Macro- and Micro-Architecture

In this regard, the concept of placing three-dimensional polymer scaffolds into spinal cord transection models has attracted immense attention in research and has achieved promising preclinical results in terms of axonal regeneration and locomotor improvement, particularly in combination with cell therapy [Refs. 4, 11, 14, 78].

Re-connection of highly-organized spinal cord tracts is imperative for functional recovery following SCI. To achieve this, scaffolds need to be engineered at a macro and micro level, affecting neurite outgrowth at a fascicular and axonal/growth cone level, respectively.

Macro-architectural, topographical cues exert considerable influence on axonal regeneration and environmental modulation. Comparing synthetic polymer scaffolds manufactured with five distinct architectural designs (i.e. cylinder, tube, channel, open-path with and without core) for implantation in a transected spinal cord, Wong et al. [Ref 95] was able to demonstrate the superiority of open path constructions. Not only did they support greater axonal regeneration, but they were also the only model that beneficially effected secondary injury progression in terms of less necrosis and scar tissue.

Scaffold microstructures, including oriented fibers, grooves and ECM molecules, are proposed to augment axonal adherence and provide precise directionality to growth, [Ref 23] thereby establishing neuronal alignment. Microgrooves, manufactured on polymer surfaces with imprinted patterns or laser etching, have been shown to enhance neurite outgrowth and contact adhesion [Refs. 96, 97]. These effects could be further improved by placing the grooves more narrowly, employing smaller groove sizes (5 μm vs. 10 μm), [Ref. 96] and coating the grooved surface with laminin [Ref 98]. The repeatedly illustrated effect of laminin gradients on axonal growth cone orientation and outgrowth [Refs. 99, 100] was also applied to hydrogel scaffolds, utilizing the gradients as chemical guidance cues [Ref. 101]. Additionally, aligned, electrospun nanofibers of polymers have achieved promising results in terms of cell attachment, migration, and alignment [Refs. 102-104]. Their three-dimensional network closely resembles the native ECM in size and architecture and thus renders them highly suitable as an environment for axonal regeneration [Ref 105].

Photolithographic surface chemical templating is a new method to direct cell adhesion and spreading. It yields a micron scale, nanometer thin, self-assembled alkylphosphonate monolayer (“SAMP”) interface, which for example can be formed from 1,4-butanediphosphonic acid, synthesized on in situ generated zirconium or titanium dioxide (TiO₂/SAMP, ZrO₂/SAMP) [Ref. 6]. This cell-adhesive chemical pattern has been demonstrated to achieve sophisticated cell alignment, which is maintained as cells grow to form a confluent monolayer across an entire two-dimensional surface [Refs. 6, 7, 106]. While it is proposed to be readily translatable to a wide array of polymer surfaces, [Refs. 7, 106] its application onto OPF hydrogels remains to be explored. Rather, without being bound to theory, it is believed that photolithography is incompatible with soft polymers (hydrogels, such as OPF) at least because removal of a photoresist is typically done in alkaline aqueous media, which could damage, or even worse, dissolve the soft polymer.

Interestingly, we observed that an open space, multichannel scaffold design resulted in regeneration of axons not only through the scaffold but on its exterior surface as well [Refs. 14, 95]. This led to the question, whether increasing surface area could further augment regeneration. On that account, our laboratory has introduced a new scaffold design: An OPF hydrogel sheet that spontaneously convolves upon hydration and provides bidirectional ridges on its surface, thereby maximizing available surface area for axonal growth (e.g., FIG. 1). Our laboratory demonstrated that laminin coated OPF+ scaffolds with closer spaced ridges (0.2 mm versus 0.4 mm versus 1 mm), thereby even further increasing the surface area, significantly improved cell attachment and led to greater neuronal alignment (Siddiqui et al. in submission). When SCs were seeded onto these ridged OPF+ sheets, they organized along the ridges over a period of 6 days. However, not all cell types behaved this way. MSCs, for instance, formed spheres upon the ridges but not on flatter surfaces, illustrating the influence of substrate nano-topographies on cell morphology. This led to the rationale of incorporating a different form of directional cues into our scaffold design, namely aforementioned micropatterned chemical interface. Our co-inventors at Princeton University have developed a method to achieve chemical patterns spaced 30 microns apart, which is closer than what is able to be physically molded. The ridges were retained on the opposite side of the OPF hydrogel, enabling the sheets to spontaneously spiral up and at the same time acting as a spacer (e.g., FIGS. 5A, 5B). As they have previously been shown to guide neurite outgrowth (Siddiqui et al.), the ridges may further provide the possibility of establishing directed neuronal spreading on the exterior surface of the scaffold when implanted in vivo.

The new scaffold design (e.g., FIG. 1) was formed of out of an OPF hydrogel sheet. The OPF sheet had TiO₂/SAMP on one surface and ridges on the other, allowing the OPF sheet to roll up into a 3D spinal configuration (e.g., a spiral) upon hydration.

### The ECM as a Therapeutic Approach for SCI

The ECM is a complex, three-dimensional, natural scaffold for tissue and cells, consisting cardinally of proteins, polysaccharides and water. It initiates essential biochemical and biomechanical stimuli for organization and morphology, including cell-cell and cell-matrix signaling, cell migration, differentiation, and adhesion [Ref. 107]. After SCI, disintegration of the ECM leads to loss of architectural support. Failure to rebuild a regenerative microenvironment impedes axonal regeneration and contributes to neuronal death, including anoikis due to a deficiency in binding substrate [Ref. 108]. The re-creation of an ECM vested with regulatory cues for survival and devoid of inhibitory ones is thus a crucial objective of bioengineering. The ECM proteins collagen, [Refs, 87, 109] fibronectin, [Refs. 90, 110] and laminin [Refs. 96, 98] have been consistently utilized in SCI due to their innate supportive properties on axon-growth, cell affinity and adhesion. Predominantly, their application has been limited to two-dimensional surface coating [Ref. 111]. However, it is now understood that the three-dimensionality and complex architecture of the native ECM provides essential biomechanical guidance and regulatory cues for cells [Refs. 112, 113]. Therefore, developing three-dimensional fibrillar networks with native tissue anisotropy through decellularization techniques has aroused immense attention in research [Ref. 113, 114]. In a recent study, a decellularized fibroblast-derived ECM was created on PET polymer micropatterned with aforementioned chemical interface [Ref. 7]. The fibroblasts were shown to assemble their ECM in alignment with the ZrO₂/SAMP pattern; neural cells consecutively spread in line with the linearly organized ECM fibrils [Ref 7]. Thus, the generation of a spatially aligned, cell-specific ECM, which is able to control the spreading of neurons across polymers and provides a full array of native proteins, was demonstrated to be feasible and offers tremendous potential in bioengineering.

### Cell Therapy

Providing both neuroprotective and neuroregenerative effects in SCI, cell therapies constitute a promising therapeutic approach. A great number of cells has been investigated, including Schwann cells (“SCs”), mesenchymal stromal cells (“MSCs”), olfactory ensheathing cells (“OECs”) [Ref. 10], oligodendrocyte progenitor cells (“OPCs”) [Ref. 115], and neural progenitor cells (NPCs) [Ref. 78], all of which were demonstrated to support axonal regeneration. With a large repertoire of potential mechanisms depending on cell type, cells have been shown to not only repopulate areas of necrotic cell loss, but also to modulate neuroinflammation, enhance plasticity, and express various growth-enhancing signaling molecules and neurotrophic factors. Intrathecal infusion, injection into the cord substance, and integration into polymer scaffolds or microspheres are feasible forms of application. As cell therapy by itself is lacking in sufficient architectural support and physical guidance cues for regenerating axons, their use with bioengineered scaffolds may offer the most promising potential for treating SCI.

### Schwann Cells

Schwann cells may be one of the most effective therapeutic cells in SCI. They produce myelin sheath within the PNS, thereby supporting axons and increasing nerve conduction velocity. In 1980, the pioneering work of Richardson and colleagues could demonstrate, that adult CNS axons are able to regenerate when provided with a PNS environment [Ref. 116]. Subsequently, SCs were shown to be a key constituent of this growth-enabling milieu [Ref 117]. Ever since, a considerable number of studies confirmed the supporting effect of SCs on axonal preservation and regeneration in the injured spinal cord, particularly when seeded onto biomaterial scaffolds [Refs. 5, 11,14]. The main contribution of exogenous SCs in spinal cord regeneration is attributed to remyelination of demyelinated axons. In fact, transplanted SCs have been demonstrated to (re)myelinate host CNS axons both within the transplant site itself and in the spared tissue rim. [Ref. 118] In addition, SCs produce a number of growth factors (“GFs”), [Ref. 119] which further makes the environment highly conducive to axonal sprouting and re-growth. When compared to polymer scaffolds seeded with NSCs [Ref 78] and MSCs, [Ref. 11] SC-loaded scaffolds led to greater axonal regeneration. SCs are proposed to augment axon guidance and trophic support, by expressing cell adhesion molecules on their surface [Ref. 120] and synthesizing extracellular components, including laminin [Ref. 121] and type IV collagen [Ref 122]. They furthermore attenuate reactive gliosis, CSPG levels and cystic cavity formation after injury, [Ref. 10, 118] thereby promoting tissue sparing. Ultimately, SCs led to improvement of functional motor recovery in animal models, [Refs. 10, 14, 118] stressing their therapeutic value.

Growth-supporting effects may further be enhanced by transducing SCs to express neurotrophins prior to transplantation [Ref 123]. As aforementioned, superior axon regeneration and remyelination as well as functional improvement could be achieved by loading OPF+ hydrogels with SCs modified to secrete high amounts of GDNF [Ref. 14].

Given the successful preclinical outcomes, clinical translation has been encouraged. Transplantation of autologous, highly purified SCs harvested from the sural nerve of SCI-patients has been proven feasible and safe [Refs. 124, 125]. However, data on the beneficial effects of these cells on functional recovery are still pending (NCT: 02354625).

### Mesenchymal Stromal Cells

Originating from the mesodermal lineage, mesenchymal stromal cells, also known as mesenchymal stem cells, are multipotent progenitors capable of differentiating into osteoblasts, chondrocytes, adipocytes, stroma cells, and skeletal myoblasts [Ref 126]. These cells can be easily and reproducibly isolated from bone marrow [Ref. 127] and adipose tissue [Ref. 128]. They can sequentially be expanded extensively by means of clinically applicable methods, [Ref. 126] rendering them amenable for autologous transplantation.

It is largely recognized, that MSCs support a growth-conducive environment following SCI by exerting trophic effects [Ref. 129]. They were shown to synthesize nerve-permissive ECM components [Refs. 12, 128] and neurotrophic cytokines, including brain derived neurotrophic factor (“BDNF”) and neural growth factor (“NGF”) [Ref 130]. Moreover, MSCs are under investigation for their unique immunoregulatory properties. Numerous studies have revealed that these cells diminish the proliferation of stimulated T-cells, [Refs. 131, 132] alter antigen-presenting cell (APC) maturation, [Ref 133] and increase circulating levels of interleukin-10—a potent anti-inflammatory cytokine [Ref 134]. Through these immunosuppressive effects, MSCs are likely capable of reducing BSCB permeability and mitigating fibrotic and glial scar formation [Refs. 8, 134]. MSCs have further been revealed to amplify the endogenous regenerative capacity of the CNS by inducing mitosis and differentiation of intrinsic stem and restorative cells as well as inhibiting apoptotic processes. [Ref. 135]. Ultimately, a systematic meta-analysis in 2013 could show that in animal models of SCI, MSCs overall significantly improved functional recovery [Ref. 9].

Clinical trials of MSCs for SCI have produced some encouraging results, confirming the safety of intravenous, intrathecal and intraspinal administration [Refs. 136, 137]. Yet, their multifaceted modulatory mechanism of action remains to be further elucidated and understood in order to optimize their therapeutic potential.

Recent research has provided a more complete understanding of the contribution of a cell-specific, self-assembled ECM that provides native neurotrophic factors and positional information [Refs. 12, 13]. Three-dimensional, naturally deposited ECM/scaffold hybrid constructs, in particular, are an emerging therapeutic approach [Ref. 114]. Our laboratory recently showed for the first time, that when co-cultured onto OPF scaffolds, human mesenchymal stromal cells (hMSCs) and SCs deposit a significantly greater amount of fibronectin than each cell type alone. Contemplating this promising finding and the advantageous effects of SCs and MSCs after SCI raises the still unexplored question, whether the combination of both cell types in a scaffold system might further increase neuroregeneration by co-establishing a growth-enhancing environment.

In conclusion, traumatic spinal cord injury is an intricate, multiphasic, and multifactorial process. Combinatorial treatments which incorporate the use of biomaterials, cell therapy, growth factors, and the ECM are necessary to achieve neuroanatomical and functional recovery [Refs. 7, 11, 14, 76, 94]. These treatments require further optimization and different combinatorial approaches need to be investigated.

### Scientific Hypothesis and Aims

Without being bound by theory, given the promising findings on OPF scaffolds, the ingenuity of surface chemical templating, and the advantageous effects of MSCs and SCs on neuroregeneration, the hypothesis can be made that: co-culture of hMSCs and SCs on TiO₂/SAMP chemically patterned OPF scaffolds enhances neurite alignment, attachment and density. This hypothesis will be achieved by four specific aims: (1) optimizing the TiO₂/SAMP chemical patterning process on OPF hydrogel sheets, (2) examining neuron attachment and alignment of dissociated DRG on TiO₂/SAMP chemically patterned OPF scaffolds, (3) investigating the efficiency of hMSCs and SCs co-cultured onto TiO₂/SAMP chemically patterned OPF scaffolds to support neurite alignment, attachment and density, and studying the ECM deposited by a hMSC and SC co-culture and its role in promoting neuronal extension.

### Material and Methods

### Experimental Design

This study outline serves as an overview of the experimental conditions, controls and crucial timepoints; details shall be described in the succeeding chapter. Aim 1: study material—TiO₂/SAMP chemically patterned OPF sheets. Aim 2: study material—TiO₂/SAMP chemically patterned OPF sheets, n=7. Controls: OPF sheets without chemical, n=3, and OPF sheets covered with unpatterned chemical, n=4. Aims 3 and 4: study material: TiO₂/SAMP chemically patterned OPF sheets with hMSC and SC co-culture, n=4. Controls: TiO₂/SAMP chemically patterned OPF sheets with hMSCs or SCs, n=4.

### Scaffold Fabrication

### OPF Polymer Synthesis

Synthesis of the OPF was done as previously described [Ref 92]. In short, poly(ethylene glycol) (“PEG”) with an average molecular weight (“Mn”) of 10,000 g/mol was first distilled in toluene and subsequently dissolved in distilled methylene chloride. A condensation reaction between PEG and distilled fumaryl chloride generated OPF with an average Mn of 16,246 g/mol. Purification of OPF from methylene chloride via rotatory evaporation ensued by recrystallization in ethylene acetate yielded OPF in a powdered form.

### Ridged OPF Sheet Fabrication

In order to produce the OPF hydrogel, 1 g of OPF powder was dissolved in 9504, deionized water. Sequentially, 0.3 g of the crosslinker N-Vinylpyrrolidinone (“NVP”) and 750 μL of a photoinitiator (Irgacure, 2959, Ciba Specialty Chemicals, Tarrytown, N.Y., USA) were added. The solution was incubated in a water bath at 37° for 30 minutes followed by centrifugation for 5 minutes at 3000 rpm. The OPF sheets were fabricated employing Teflon molds, on which a spacer was used to create sheets of 0.08 mm thickness. The molds contained micro-grooves 100 μm in depth and spaced 1 mm apart in parallel lines to create ridges (e.g., ridges 108 of FIG. 1). The OPF hydrogel solution was pipetted onto one concentrated central area of the Teflon mold; a glass slide was then slowly placed on top in order to obtain an even spread of the liquid polymer. Clamps were placed on all four sides, fixating the glass slide in place and removing any air bubbles. One hour of exposure to UV light (365 nm) at an intensity of 8 mW/cm² led to the polymerization of the OPF scaffolds, which were thereafter allowed to cure overnight. The next day the glass plates were detached from the molds with the polymer adhering to the glass. In order to separate the sheets from the plates more easily, they were partially hydrated with a few drops of water. Afterwards, the OPF sheets were cut into 4 mm by 4 mm squares.

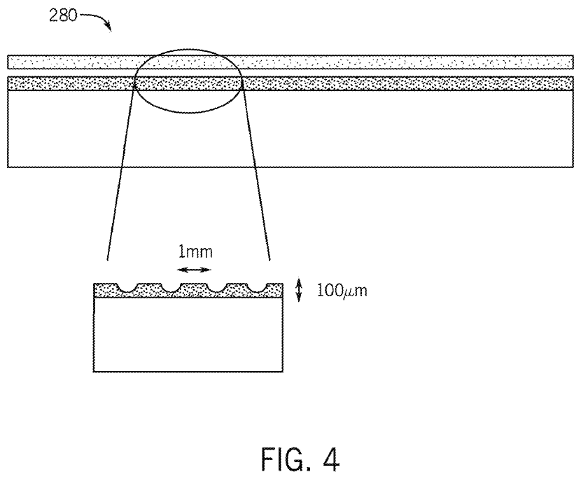

FIG. 4 illustrates a micro-grooved Teflon® mold 280 for OPF sheet fabrication. In order to generate scaffolds with ridges spaced 1 mm apart and 100 μm in depth, molds were utilized that are constituted of glass, grooved Teflon® and Plexiglas™. As shown, the mold 280 has a plurality of cavities, which corresponds with the plurality of ridges (e.g., the plurality of ridges 108) when the OPF sheet is cured.

FIGS. 5A, and 5B shows the spontaneous furling (or rolling up of) the OPF sheet created using the mold of FIG. 4. The ridges (spaced 1 mm apart) on the OPF sheet can, upon hydration of the OPF sheet, cause the OPF sheet to furl or roll up.

### OPF Hydrogel Chemical Patterning

The OPF hydrogel was micropatterned via surface chemical templating so as to promote cell adhesion and alignment along the orientation of the pattern. Briefly, shadow masks and a chemical solvent deposition-based sequence were used to generate 30 μm by 30 μm stripes (FIGS. 6A and 6B). Kapton shadow masks were produced with a Heidelberg DWL 66 laser writer (FIG. 6C).

FIG. 6A shows light microscopic images of TiO₂/SAMP 30×30 micron stripes (e.g., each TiO₂/SAMP strip having a 30 μm thickness) on an OPF surface at 10× magnification, while FIG. 6B shows light microscopic images of TiO₂/SAMP 30×30 micron stripes on an OPF surface at 2.5× magnification. FIG. 6C shows the dimensions of the Kapton® shadow mask for the chemical pattern.

In order to fabricate TiO₂/SAMP coating that is robustly bonded to the polymer substrate, two working steps were necessary: preparing the solutions and debossing OPF hydrogel. Prior to preparing the solutions, glass slides were cut into 1 cm by 3.5 cm rectangles in order to fit into 20 ml scintillation vials. An AtmosBag (Aldrich®, Inflatable Polyethylene Isolation Chamber with Zipper lock, 50 L volume, Z530204-1EA) with two built-in gloves was set up as a working space and purged with nitrogen. In order to achieve low moisture and oxygen levels, 4-5 vacuum/nitrogen purge cycles were performed. Afterwards, the bag was inflated to an operational level that allows easy insertion of hands.

Since titanium (IV) isopropoxide (Acros Organics®, CAS: 546-68-9) is oxygen and moisture-sensitive (and 12-(phosphonododecyl)phosphonic acid (Sigma-Aldrich®, St. Louis, Mo., USA, CAS: 7450-59-1), while not apparent to be oxygen and moisture sensitive, but to exercise caution), all solutions were prepared inside the glove bag in 20 ml glass scintillation vials. In order to produce the titanium solution, 30 μL of titanium (IV) isopropoxide were pipetted into 5 ml of anhydrous toluene (Alfa Aesar®, Ward Hill, Mass., USA, CAS: 108-88-3), a hydrophobic solvent. 1 mg of 12-(phosphonododecyl)phosphonic acid was added to 5 ml of anhydrous toluene, preparing the phosphonic acid solution. The solutions were capped tightly before being taken out of the glove bag and were applied in the debossing step straightaway.

Once hydrated, OPF sheets were carefully placed onto the glass slides. Excess water was removed by blotting the OPF surface with filter paper. The Kapton® shadow mask was placed onto the OPF surface tautly so as to prevent the stripes from sticking together. Thereafter, the OPF-mask composite was heated on a hotplate at 30° C. under a flat-bottomed 200 g weight for 15 seconds. The substrate was then immersed into the vial filled with the titanium isopropoxide solution for 30 seconds and sequentially washed with toluene 3 times for 10 seconds each. It was then heated at 30° C. for 30 seconds to generate the cross-linked TiO₂ base layer that is bound to the polymer surface. The composite was allowed to cool for 15 seconds before being dipped into the phosphonic acid solution. It was left submerged overnight to bind a self-assembled monolayer of phosphonic acid across its surface in patterned strips of 30 μm wide stripes of TiO₂/SAMP with 30 μm wide intervals (30 μm×30 μm pattern) (FIG. 4). The following day, the substrate was rinsed sequentially with toluene, isopropanol, and water three times each. The OPF sheet was detached from the mask. The scaffold was placed onto a piece of Kapton®, heated at 30° C. for 30 seconds and subsequently stored in double-distilled water until used.

The Kapton® shadow masks were cleaned by sonicating them for 15 seconds in a low concentration solution of potassium. Afterwards, they were sonicated three more times in deionized water for 5 minutes each and were left to air-dry. In this way, the masks were reusable until they frayed.

### OPF Sheet Preparation for Culture

The unique property of OPF sheets to spontaneously roll up when hydrated necessitates pinning them down for culturing. First, 4 mm by 4 mm squares were cut in half using a blade. With ridges facing downwards they were pinned flat in culture dishes containing a layer of silicone elastomer (SYLGARD® 184 Silicone Elastomer Kit: base and curing agent, 180409, Electron Microscopy Sciences). As per manufacturer's instructions the Sylgard® 184 base and curing agent were mixed in a 10:1 ratio by weight and filled into petri dishes, where the mixture cured to a flexible elastomer. Insect pins, one per corner, were inserted in an angle so as to not obstruct the field of view on the scaffold (FIG. 5). For the purpose of sterilization, affixed sheets were alternately washed with 70% isopropanol and Dulbecco's Phosphate Buffered Saline (DPBS, Corning®, Manassas, Va., USA). Thereafter, the sheets received two consecutive washes in DPBS (adding up to a total of 7 washes for 5 minutes each) to remove any residual isopropanol.

Scaffolds that were to be seeded with neurons from dissociated dorsal root ganglia (“DRG”) only, were subsequently coated with 100 μl of fibronectin (10 μg/ml; Schwarzbauer laboratory, Princeton University). Scaffolds for hMSC and SC cultures were directly submerged in Dulbecco's Modified Eagle Medium/F12 medium (DMEM/F12). All dishes (coated and uncoated) were incubated at 37° C. in 5% CO₂. Before further usage, the fibronectin solution was removed and sheets were washed 2 times with DPBS followed by 2 washes with Minimal Essential Medium (MEM, Gibco®, Grand Island, N.Y., USA) for 5 minutes each.

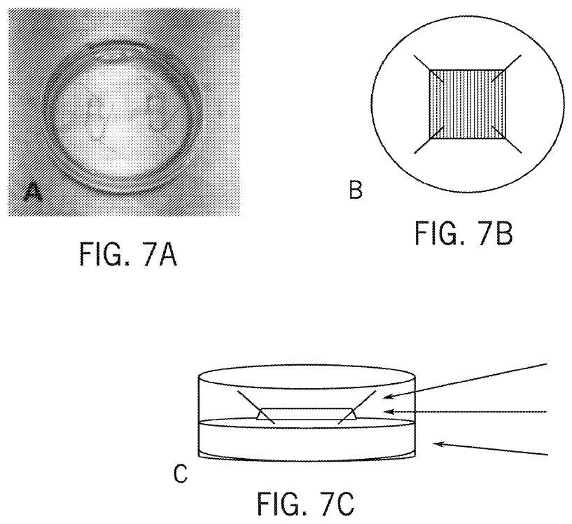

FIG. 7A-7C illustrates the scaffold preparation before cell culturing. OPF sheets were pinned flat in 35 mm culture dishes on a layer of silicon elastomer by use of insect pins (A, B & C). For example, FIG. 7A shows an image of the oligo(poly(ethylene glycol) sheet that has been unfurled (unrolled), and mechanically constrained with the ridges faced towards the petri dish. FIG. 7B is a top view illustration of the configuration of FIG. 7A, and FIG. 7C is a front view illustration of the OPF sheet of FIG. 7B.

### Human Mesenchymal Stem Cell Isolation and Culture

hMSCs were isolated from adipose tissue via an adipose biopsy. The cells were subsequently expanded and cryopreserved in the Mayo Clinic Human Cellular Therapy Laboratory, as previously described [Ref. 138]. hMSCs were cultured at 37° C. in 5% CO₂ in advanced MEM media (Gibco®, cat #12492021) containing Human platelet lysate (Lawson®, Mill Creek), Glutamax (100 units/ml; Gibco®, cat #35050061), Penicillin/Streptomycin (100 units/ml; Gibco®, cat #15240062) and Heparin (Novaplus®, cat #6009596). The release criteria for MSCs comprised the cellular phenotype (CD90+, CD105+, CD73+, HLA Class I+, CD44+, CD14−, CD45− and HLA DR), negative mycoplasma detection, culture sterility, and cytogenetic analysis.

### Human Mesenchymal Stem Cell Plating

When approximately 80-90% confluency was reached, hMSCs were detached from the flasks by adding TrypLE (Gibco®, cat #12604013) for 5 minutes at 37° C. Sequentially, media was added in order to deactivate the enzymatic activity of TrypLE and cells were centrifuged at 1000 rpm for 5 minutes. Afterwards, the supernatant was discarded and hMSCs were resuspended in media. Using a haemocytometer the cells were counted and OPF scaffolds were seeded at a density of 200,000 cells (suspended in 100 μl of media). The dishes were incubated for 1 hour to allow for attachment of cells to the polymer surface before being fully suspended in media.

### Schwann Cell Isolation and Culture

All surgical and care procedures involving animals were approved by the Mayo Clinic Institutional Animal Care and Use Committee (“IACUC”), in strict accordance with the Institute for Laboratory Animal Research (“ILAR”).

Primary rat Schwann cells were obtained from the sciatic nerves of Sprague Dawley pups at postnatal day 5, according to a previously described method [Ref 4]. Briefly, the sciatic nerves from both legs were surgically isolated under aseptic conditions, stripped of connective tissue and epineurium and cut into 1 mm sections. They were treated enzymatically for 45 minutes with 0.25% trypsin EDTA (Mediatech Inc., Herndon, Va., USA) and 0.03% collagenase (Sigma®, St. Louis, Mo., USA) in Hank's Balanced Salt Solution (HBSS). The cells were mechanically dissociated by pipetting up and down after which they were centrifuged for 5 minutes at 1000 rpm. The supernatant was removed before SCs were resuspended in DMEM/F12 with L glutamine (Gibco®, cat #1951120) supplemented with Forskilin (204), Neuroregulin (10 ng/ml; Life Sciences, cat #4711), 10% fetal bovine serum (FBS), and 1% Penicillin/Streptomycin.

### Schwann Cell Plating

SCs were plated onto OPF hydrogel sheets (200,000 cells per scaffold) in a similar method to hMSCs (described above). However, SC media was used containing DMEM/F12 with L glutamine supplemented with Forskilin (204), Neuroregulin (10 ng/ml), 10% FBS, and 1% Penicillin/Streptomycin.

### Co-Culture

SCs and hMSCs were co-cultured in a 50:50 ratio (100,000 SCs and 100,000 hMSCs per scaffold) and media containing DMEM/F12 with L glutamine, Forskilin (204), Neuroregulin (10 ng/ml), 10% FBS, and 1% Penicillin/Streptomycin was used. Once dissociated DRG were added to the culture, 15% AN2 media was employed. It was produced by mixing MEM with 15% calf bovine serum (CBS; HyClone®, Logan, Utah, USA), 20% Glucose (7 mg/ml; Sigma Aldrich®) and L-glutamine (200 mM; Gibco®). Nerve growth factor (NGF, 5 ng/ml in MEM with 2 mg/ml cytochrome c as a carrier protein; Harlan Bioproducts for Science®, Indianapolis, Ind., USA) was added fresh to the media before usage. If SCs were part of the culture, Forskilin (204) and Neuroregulin (10 ng/ml) were added to 15% AN2 media.

### Dorsal Root Ganglion Retrieval

Neurons were obtained from dissociated DRG isolated from Sprague Dawley rats at embryonic day 15. Aseptic precautions and sterile techniques were applied in all steps.

### Large Dissection

The gestating rat was euthanized by gradual carbon dioxide exposure. After sterilizing the abdomen with 70% ethanol, an incision was made from the sternum to the urethra, solely cutting the top layer of the skin. The abdomen was exposed by drawing down the skin on either side using two homeostatic forceps and sequentially cut open with scissors. Particular attention was paid to not cut the bowel as this can be a source of bacterial contamination. The uterus was cut at its base, carefully removed and placed into a sterile glass petri dish. The succeeding steps were conducted in a laminar flow hood, starting with an incision from the top of one uterine horn toward the uterine base. Each prenatal rat was then separated from its placenta and amniotic sac and placed into a dish filled with Leibovitz's-15 media (L-15) (Gibco®, Grand Island, N.Y., USA).

### Microdissection

The microdissection was carried out under a dissecting microscope (Carl Zeiss, Jena, Germany) and commenced with the removal of the head. Sequentially, an incision was made on either side from between the tail and the hipbone to above the shoulder with micro scissors. At this time, it was feasible to separate the vertebral column from the rest of the body; any residual organs or large blood vessels were removed. With the ventral side tending upwards, a horizontal incision was made along both sides of the vertebral column so as to remove the vertebral bodies. As a result, the spinal cord was exposed, which could then be removed by carefully pulling at its rostral end utilizing micro forceps. The DRG were removed from the isolated cord, submerged in L-15 media and further used for neuronal dispersal.

### Dissociated DRG Plating

In order to plate dissociated DRG neurons onto the OPF sheets, 15% AN2 media was prepared (specified above). With approximately 1.5×10⁶ neurons isolated per ganglion and 30-40 DRG obtained per prenatal rat, the DRG of about 5 spinal cords were required for one experimental set. The collected DRG were pipetted into a plastic centrifuge tube. Beforehand, the pipette's plastic tip had been washed with AN2 media so as to avoid sticking of the DRG to the plastic. The tube was spun at 2000 rpm for 10 minutes and the supernatant was removed afterwards. For the purpose of dissociation, the DRG were gently resuspended in 1 ml of a filter-sterilized trypsin solution, consisting of 0.25% trypsin (Worthington Biochemical Corporation®, Lakewood, N.J., USA) in HBSS, and incubated for 30 minutes at 37° C. in 5% CO₂. Thereafter, the cells were centrifuged once more at 2000 rpm for 10 minutes. After removing the supernatant, 1 ml of 15% AN2 media was added and the cells were mechanically dissociated by pipetting the media up and down. A Pasteur pipette was employed, whose opening had been narrowed to <200 μm with fire polishing, in order to guarantee dissociation. 100 μL (containing approximately 50,000 neurons) of resuspended cells were then pipetted onto each polymer sheet. The scaffolds were subsequently placed into the incubator for 1 hour, allowing the cells to become adherent to the polymer surface. Afterwards, 1-2 ml of 15% AN2 media were added so as to fully cover the cells and the plates were incubated overnight.

In order to attain purified neuron dispersal, contaminative non-neuronal supporting cells needed to be removed. On that account, scaffolds cultured only with DRG neurons were treated the following day with the antimitotic reagent 5-fluoro-2-deoxyuridine (“FUDR”) and uridine (204 each; Sigma®, F0503 Lot SLBL9184V and U3003 Lot 061K12165 respectively, St. Louis, Mo., USA) in 15% AN2 media supplemented with NGF (5 ng/ml). After 3 days the purifying solution was replaced by fresh 15% AN2 media containing NGF (5 ng/ml). Since FUDR and uridine would most likely cause undesired cell death of hMSCs and SCs, scaffolds cultured with these cells were not treated.

The cells were fixed 2 days later by submerging them in 4% paraformaldehyde for 30 minutes. Thereafter, the sheets were washed three times with PBS for 5 minutes each.

### Immunocytochemistry

### Antibody Staining

Cell-seeded scaffolds were blocked for 30 minutes with 100 μl of blocking solution containing PBS with 0.3% Triton X-100 and 5% normal donkey serum (NDS; Millipore Chemicon®). The primary antibody (Table 1) was diluted in 0.3% Triton X-100 PBS with 5% NDS and 100 μl were pipetted onto each scaffold. The dishes were incubated at 4° C. overnight in a humidified slide box to prevent scaffolds from drying out. The next day, OPF sheets were washed 3 times in PBS with 0.1% Triton X-100 for 5 minutes each. The scaffolds were sequentially incubated in secondary antibody (Table 1) diluted in 5% NDS and PBS with 0.3% Triton X-100 for 60 minutes at room temperature. Afterwards, samples were washed in PBS with 0.1% Triton X-100 3 times for 5 minutes each followed by 2 washes solely in PBS and were then stored at 4° C. Before imaging, polymer sheets were freed from insect pins and gingerly placed onto glass slides with the cell surface facing upwards. After applying Slow Fade Gold Antifade Reagent with DAPI nuclear stain (Molecular Probes®, Eugene, Oreg., USA), glass coverslips were mounted on top and slides were stored in the dark.

TABLE 1
Primary and secondary antibodies, their targets and dilutions.

| | | | |
|---|---|---|---|
| | | Dilution | |
| Antibody | Target | factor | Manufacturer |

Primary Antibody:

| | | | |
|---|---|---|---|
| Mouse anti-β-tubulin | Neurons | 1:300 | Millipore MAB1637 |
| Rabbit anti-S-100 | Schwann | 1:4000 | Dako |
| | cells | | |
| Rabbit anti-fibronectin | Fibronectin | 1:100 | R184 was gifted from |
| | | | Princeton |
| Mouse anti-laminin | Laminin | 1:100 | 2E8 was purchased from |
| | | | DSHB (Developmental |
| | | | studies hybridoma bank) |

Secondary Antibody:

| | | | |
|---|---|---|---|
| Donkey anti-mouse | Mouse 1°Ab | 1:600 | Millipore |
| Cy3 | | | |
| Donkey anti-rabbit | Rabbit 1°Ab | 1:600 | Millipore |
| Cy5 | | | |

### Image Acquisition and Analysis

All fluorescent images were acquired using a Zeiss Axio Observer Z-1 microscope (Carl Zeiss, Inc., Oberkochen, Germany) with the aid of ZEN 2 (blue edition) imaging software (Zeiss). Pictures were taken by use of an Axiocam 503 mono (Zeiss) with a lens of 0.3 numerical aperture. Z-stacks with 5 slices at magnification of 10× and 20× were acquired employing an EC Plan-Neofleur 10×/0.30 ph1 objective. In order to minimize bias, 4 representative areas on each scaffold were captured in a systematic manner. Proceeding from the center position one field of view was moved up or down followed by one field of view left or right, yielding a total of 4 quadrants.

All fluorescent images were analyzed with the aid of ImageJ (NIH) software. Staining intensities were quantified by measuring the mean grey values of β-tubulin and fibronectin on each representative field of view. In order to adapt different exposures, each mean grey value was normalized to DAPI fluorescent intensity per cell. An average value derived from the 4 quadrants was calculated for each scaffold. For the purpose of neuron quantification, all cell nuclei (DAPI, blue) that were associated with neurites (β-tubulin, red) were counted on each quadrant and an average value was calculated per OPF sheet.

### Statistical Analysis

The number of neurons and fluorescent intensities were described as the average value ± standard error of the mean (“SEM”) with the aid of GraphPad Prism 7 (GraphPad Software, Inc., USA). One-way analysis of variance (one-way ANOVA) and Tukey's post-hoc test were run to test for significant differences between experimental groups after Gaussian distribution was determined with the Shapiro-Wilk test. Statistical significance was defined as p<0.05. The degrees of significance are indicated as follows: p<0.05 (*), p<0.01 (**), p<0.001 (***), and p<0.0001 (****).

### Results

### Application of TiO₂/SAMP Chemical Pattern onto OPF Hydrogel Sheets

OPF sheets were chemically micropatterned to obtain 30 μm by 30 μm stripes (e.g., stripes having a uniform width of 30 μm) across the hydrogel (OPF) surface. The substrates were debossed by use of shadow masks and exposure to a titanium dioxide (TiO₂) precursor and heat yielded the cross-linked TiO₂ base layer bound to the polymer surface. Incubation in phosphonic acid assembled the SAMP on the oxide (TiO₂/SAMP) in striped patterns.

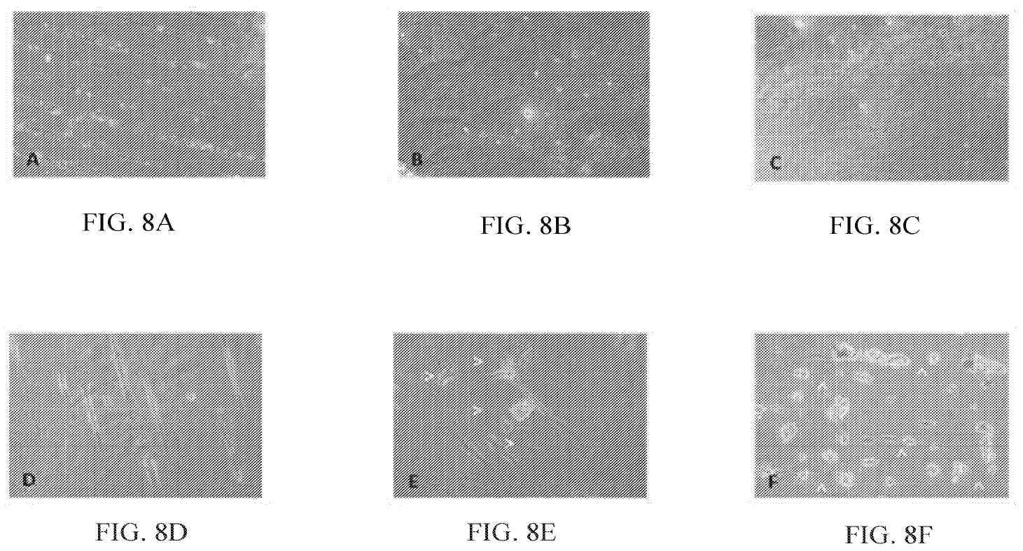

Different temperatures (20° C., 30° C. and 40° C.) were tested for the chemical patterning process. Heating scaffold-mask composites with 30° C. generated well-defined stripes across the hydrogel surface, which is shown in FIG. 8A. As opposed to this, stripes turned out disseminated and indistinct when 20° C. and 40° C. were used as heating temperatures, which is shown in FIGS. 8B, and 8C, respectively.

Comparing different concentrations of titanium isopropoxide of 3 μL/5 mL (e.g., 3 of −98% titanium (IV) isopropoxide in 5 mL anhydrous toluene) and 30 μL/5 mL (e.g., 304, of −98% titanium (IV) isopropoxide in 5 mL anhydrous toluene), showed that the higher concentration yielded continuous and uniform stripes along which cells were able to elongate. For example, FIG. 8D shows an image of the resulting titanium dioxide stripes using the 30 uL/5 mL concentration of titanium dioxide, with cells elongated. Conversely, stripes of the 3 μL/5 mL concentration of titanium isopropoxide used to form the titanium dioxide layer led to the stalled (or otherwise thwarted) elongation of cells along the pattern. For example, in FIGS. 8E and 8F the titanium dioxide layer was created using the 30 μL/5 mL concentration.

FIGS. 8A-8F show microscopic images of chemically micropatterned hydrogels formed at different temperatures and using different titanium isopropoxide concentrations. These microscopic images were captured at 10× magnification. FIG. 8A shows confined and precise stripes across the polymer surface, possibly due to the 30° C. heating temperature. FIG. 8B shows a polymer surface of a polymer heated at 20° C. FIG. 8C shows a polymer surface of a polymer heated at 40° C. FIG. 8D shows a polymer surface treated using a 30 μL/5 mL titanium isopropoxide concentration, and having hMSCs elongated on the stripes. FIG. 8D was taken three days after culturing the hMSCs on the surface. FIG. 8E shows a polymer surface treated using the 3 μmL/5 mL titanium isopropoxide concentration, and having hMSCs on the surface. FIG. 8F shows a polymer surface treated using 3 μL/5 mL the titanium isopropoxide concentration, and having SCs on the surface. FIGS. 8E and 8F show arrested extension of hMSCs and SCs, respectively.

To conclude, the application of TiO₂/SAMP patterned surface chemistry onto OPF sheets is feasible, with 30° C. heating temperature and 30 μl/5 ml titanium isopropoxide concentration as an optimal parameters for the process.

### Chemically Micropatterned OPF Sheets Direct Neural Cell Alignment

To determine the capability of chemically patterned OPF scaffolds to promote neurite alignment, dissociated neurons from DRG were seeded onto the 30 μm×30 μm TiO₂/SAMP patterned sheets with fibronectin coating. Two control scaffolds were used for comparison: OPF without chemical (fibronectin coating only) and OPF covered with unpatterned chemical (full TiSAMP).

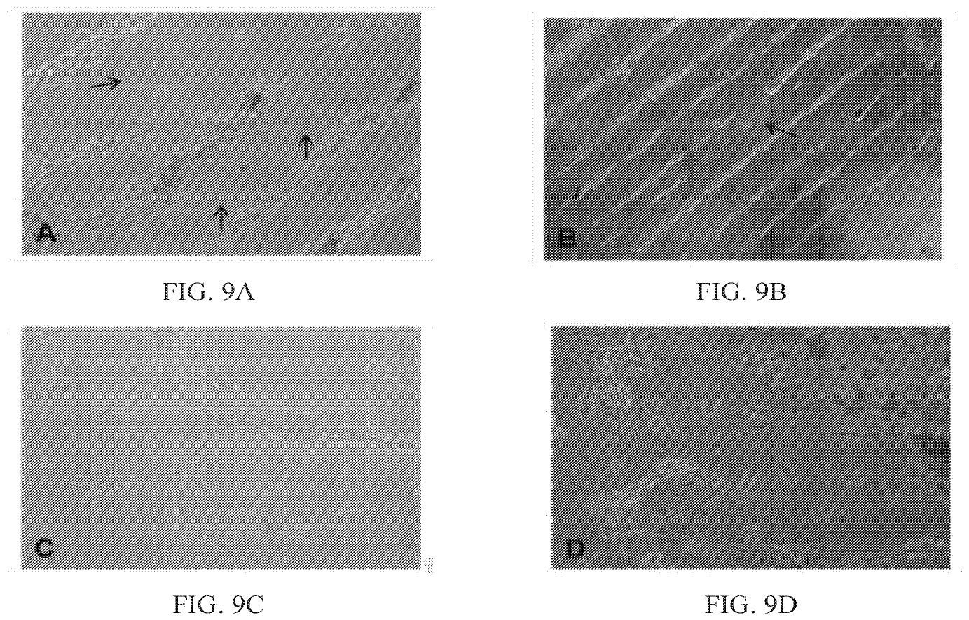

Light microscopic images were captured after 3 days of culture. On the patterned scaffolds neurons were shown to preferentially adhere to the surface chemistry and elongate in alignment with the stripes (FIGS. 9A and 9B), the stripes providing micro-topographical cues. Scarcely any cells were observed in the chemistry-free intervals. Sometimes neurons were able to extend across unpatterned intervals and elongate along adjacent stripes, which is shown by the black arrows within FIGS. 9A and 9B. By contrast, neurons grew in random orientation on unpatterned control scaffolds (FIGS. 9C and 9D).

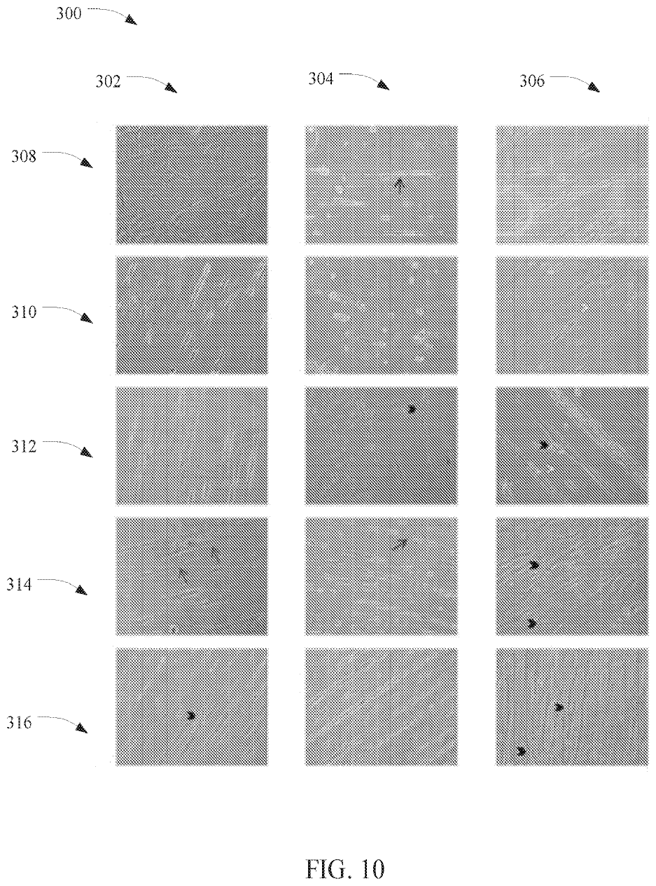

FIGS. 9A-9D shows dissociated DRG neurons aligned on chemically patterned OPF with fibronectin coating, chemically unpatterned OPF, and chemically lacking OPF after 3 days of culture. FIG. 9A shows a microscopic image of a 30 μm×30 μm TiO₂/SAMP patterned OPF scaffolds at 10× magnification with a fibronectin coating on the OPF sheet (e.g., in the channels and on the TiO₂/SAMP layer). FIG. 9B shows a microscopic image of a 30 μm×30 μm TiO₂/SAMP patterned OPF scaffolds at 2.5× magnification. FIGS. 9A and 9B show that the surface promoted a high degree of neurite alignment. FIG. 9C shows a microscopic image of an OPF scaffold having no surface chemicals (e.g., no TiO₂/SAMP), taken at 10× magnification. FIG. 10D shows a microscopic image of an OPF scaffold having surface chemicals (e.g., TiO₂/SAMP), but having no chemical pattern (e.g., no channels), taken at 10× magnification. FIGS. 9C and 9D, both controls, show neurites having no spatial directionality.

### The Impact of Co-Culturing hMSCs and SCs on Neuron Attachment and Density Neuron Attachment and Alignment

To investigate the efficiency of scaffolds with different cell types in supporting neuronal attachment and alignment, dispersed DRG neurons were plated onto chemically patterned OPF sheets that had been cultured with both hMSCs and SCs. Scaffolds seeded with either hMSCs or SCs alone and scaffolds with fibronectin coating (DRG neurons only) were used as controls.

Before neurons were cultured on the sheets, it was shown that SCs and hMSCs aligned according to the orientation of the TiO₂/SAMP pattern during 6 days of culture (FIG. 10). Neurons were either plated onto the cell-monolayer or a fibronectin coat for 4 days (FIG. 10). Immunofluorescent images were acquired and quantification was done by counting the number of neurons on and in between the patterned chemistry (Table 2).

FIG. 10 shows a series of microscopic images 300 of cells cultured on chemically patterned OPF sheets. FIG. 10 specifically shows a column 302 representing all images within FIG. 10 being cultured with human mesenchymal stem cells, a column 304 representing all images within FIG. 10 being cultured with Schwann cells, and a column 306 representing all image within FIG. 10 being co-cultured with both human mesenchymal stem cells and Schwann cells. FIG. 10 also shows rows of microscopic images taken at different time intervals of culturing, and including or not including rat dorsal root ganglia (“DRG”). For example, FIG. 10 also includes a row 308 of images not including the addition of DRG and taken after 1 Day of culturing. Row 310 is a row of microscopic images within FIG. 10 not including the addition of DRG and taken after 3 days of culturing. Row 312 is a row of microscopic images within FIG. 10 that includes the addition of DRG and taken after 6 days of culturing. Row 314 is a row of microscopic images within FIG. 10 that includes the addition of DRG and taken after 8 days of culturing. Row 316 is a row of microscopic images within FIG. 510 that includes the addition of DRG and taken after 10 days of culturing.

FIG. 10 generally shows hMSCs, SCs, and dispersed neurons cultured onto chemically patterned OPF sheets aligned along the orientation of the chemically patterned stripes. hMSCs and SCs as well as neurons attached to and aligned along the 30×30 μm stripes during 10 and 4 days of culture, respectively. Notably, the cells' cytoskeletons were observed to become thinner as the cells elongated along the pattern, as indicated by → of column 304. Interestingly, the cell morphology may differ depending on the thickness of the stripes (which may vary due to discrepancies in manufacturing), with thicker cell bodies found on thicker stripes and vice versa, as indicated by → of column 304. Cells started to grow to confluence during culture, while maintaining the orientation of the pattern, as indicated by the black chevrons (e.g. [symbol] ). Discontinuity in chemical pattern (which may appear due to discrepancies in manufacturing) led to stalled cell elongation along the stripes, as indicated by > of column 306.

Results showed that the neurons predominantly attached to the chemical on all investigated scaffolds, with the average percentage of neurons on the chemical ranging from 90.05% (SCs) to 93.38% (co-culture). The neurons were observed to spread in the direction of the chemical stripes on all experimental sheets. These findings indicate a high degree of neuron alignment in accordance with the patterned surface chemistry that is irrespective of the growth condition.

The number of neurons on the chemical was found to be highest on SC-cultured (50.29±15.07) and second highest on co-cultured scaffolds (40.88±5.45). Both groups significantly increased the number of neurons on the chemical when compared to scaffolds seeded with neurons only (10.51±2.52) (p=0.009 and p=0.049 respectively). These results suggest that seeding OPF sheets with hMSC and SC co-culture or SCs alone enhances neuron attachment.

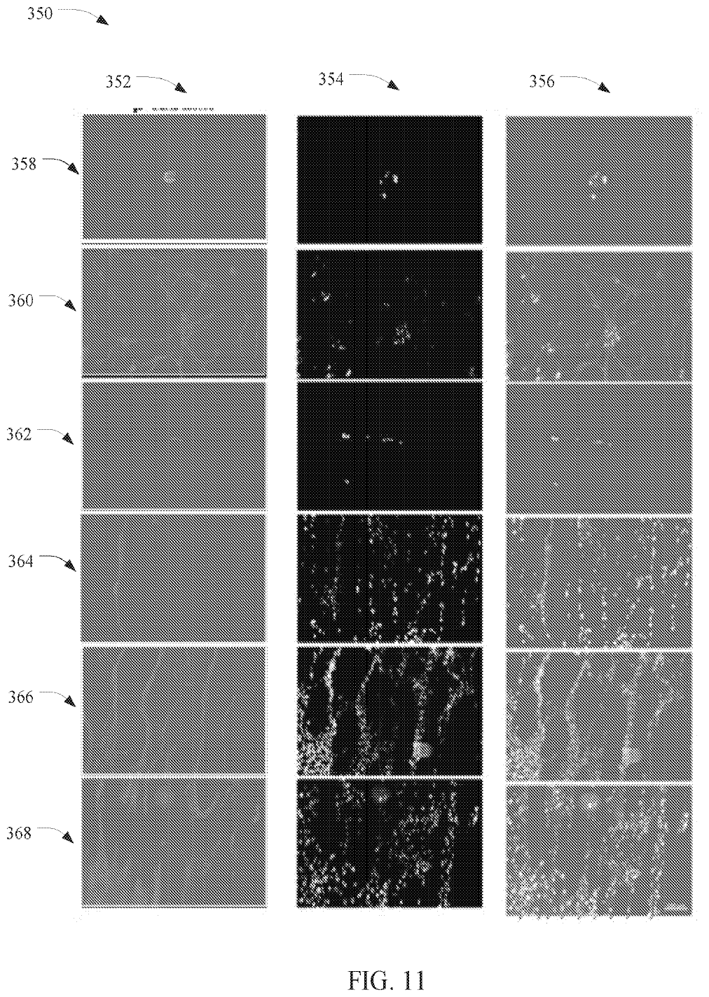

FIG. 11 shows a series of immunofluorescent images 350 stained with different staining targets for various OPF sheets. For example, column 352 represents all images within FIG. 11 being stained for β-tubulin, where the β-tubulin appears as red. Column 354 represents all images within FIG. 10 being stained for the cell nuclei (e.g., using 4′,6-diamidino-2-phenylindole “DAPI”), where the cell nuclei appear as white. Column 356 is the superimposed combination of the images of columns 352 and 354. Thus, column 356 represents the combination of both staining regimens, such that the images within column 356 have red and white portions (e.g., superimposed images of the images of columns 352, 354 for a given row). Row 358 is a row of immunofluorescent images within FIG. 11 not including a chemical patterning on the OPF surface (e.g., no TiO₂/SAMP layer), but including a fibronectin coating on the OPF surface, and cultured with DRG neurons. Row 360 is a row of immunofluorescent images within FIG. 11 including chemical treatment across the entire OPF surface (e.g., a TiO₂/SAMP layer), and a fibronectin coating, and cultured with DRG neurons. Row 362 is a row of immunofluorescent images within FIG. 11 including a chemical patterning (e.g., a TiO₂/SAMP layer) on the OPF surface, having a 30 μm width repeating pattern (e.g., 30 μm×30 μm), as discussed above, and a fibronectin coating, and cultured with DRG neurons. Row 364 is a row of immunofluorescent images within FIG. 11 including a chemical patterning (e.g., a TiO₂/SAMP layer) on the OPF surface, having a 30 μm width repeating pattern (e.g., 30 μm×30 μm), as discussed above, and cultured with human mesenchymal stem cells on the chemical pattern, and DRG neurons cultured on the human mesenchymal stem cells. Row 366 is a row of immunofluorescent images within FIG. 11 including a chemical patterning (e.g., a TiO₂/SAMP layer) on the OPF surface, having a 30 μm width repeating pattern (e.g., 30 μm×30 μm), as discussed above, and cultured with Schwann cells on the chemical pattern, and DRG neurons cultured on the Schwann cells. Row 368 is a row of immunofluorescent images within FIG. 10 including a chemical patterning (e.g., a TiO₂/SAMP layer) on the OPF surface, having a 30 μm width repeating pattern (e.g., 30 μm×30 μm), as discussed above, and cultured with a co-culture of human mesenchymal stem cells and Schwann cells on the chemical pattern, and DRG neurons cultured on the co-culture.

FIG. 11 generally shows immunofluorescent pictures demonstrating neurite alignment promoted by chemically patterned OPF. With regard to immunocytochemistry, the samples were stained for β-tubulin (neurons, red) and DAPI (cell nuclei, white). Images were captured at 10× magnification. An aligned neurite outgrowth of the dispersed neurons along the chemical stripes was observed. No spatial directionality was found on unpatterned scaffolds (TiSAMP full and no chemical).

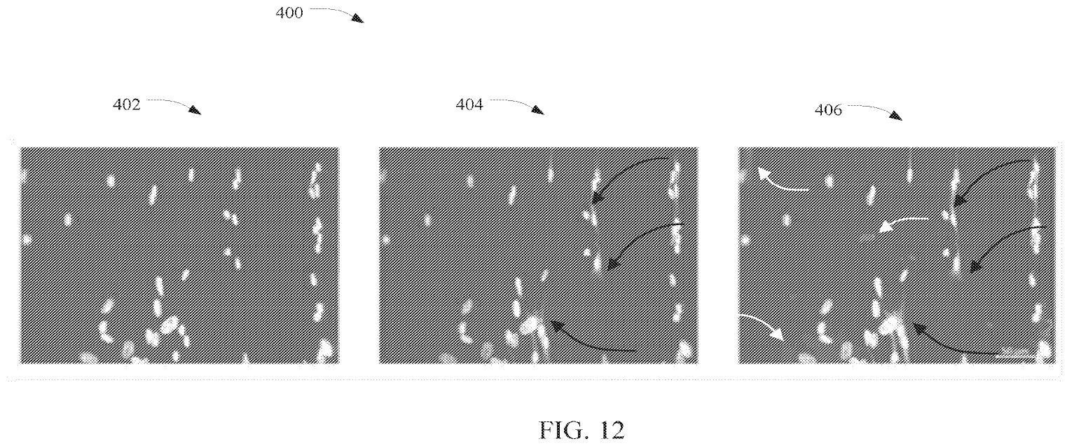

Since cells can outcompete each other for resources, the presence of both hMSCs and SCs in co-culture conditions was confirmed by visualizing the different cell types with immunocytochemistry after 10 days of culture. The samples were stained with S-100 antibody which selectively labels Schwann cells (green, shown in the black arrows in FIG. 12) and β-tubulin labeling neurons (red, shown with the black arrows in FIG. 12). All remaining cells that were neither associated with green nor red represented hMSCs.

FIG. 12 shows immunofluorescent images confirming the presence of hMSCs, SCs, and DRG neurons in co-cultures. Pictures were taken at 20× magnification. With regard to immunocytochemistry, the scaffolds were stained for DAPI (cell nuclei, white), S-100 (SCs, green) and β-tubulin (neurons, red) in order to demonstrate the presence of all three cell types (hMSCs, SCs and neurons) in co-culture samples. Specifically, FIG. 12 shows a series of immunofluorescent images 400 with different staining targets for various OPF sheets. Immunofluorescent image 402 shows staining of cell nuclei (e.g., using 4′,6-diamidino-2-phenylindole “DAPI”), where the cell nuclei appear as white, of a co-culture of both human mesenchymal stem cells and Schwann cells, and DRG neurons. Immunofluorescent image 404 shows staining with S-100, appearing as green (shown in FIG. 12 with black arrows), of a co-culture of both human mesenchymal stem cells and Schwann cells, along with DRG neurons. Immunofluorescent image 406 shows staining of β-tubulin, staining for cell nuclei, and staining with S-100 for the co-culture of human mesenchymal stem cells, Schwann cells, and DRG neurons. The β-tubulin appears as red (show in FIG. 12 with white arrows) and indicates the activity of the DRG neurons, while the green staining (shown in FIG. 12 with black arrows) indicates the activity of the Schwann cells.

TABLE 2
Number and percentage of neurons on the chemical and number of neurons off the
chemical at different growth conditions. Data are presented as mean ± SEM.

| | DRG neurons only | hMSCs | SCs | Co-culture |
|---|---|---|---|---|
| | (n = 7) | (n = 4) | (n = 4) | (n = 4) |
| % of neurons on the | 90.07 ± 3.84% | 92.45 ± 3.48% | 90.05 ± 1.35% | 93.38 ± 0.57% |
| chemical | | | | |
| Number of neurons | 10.51 ± 2.52 | 21.40 ± 5.50 | 50.29 ± 15.07 | 40.88 ± 5.45 |
| on the chemical | | | | |
| Number of neurons | 1.42 ± 0.40 | 2.00 ± 0.98 | 5.29 ± 1.31 | 2.94 ± 0.49 |
| off the chemical | | | | |

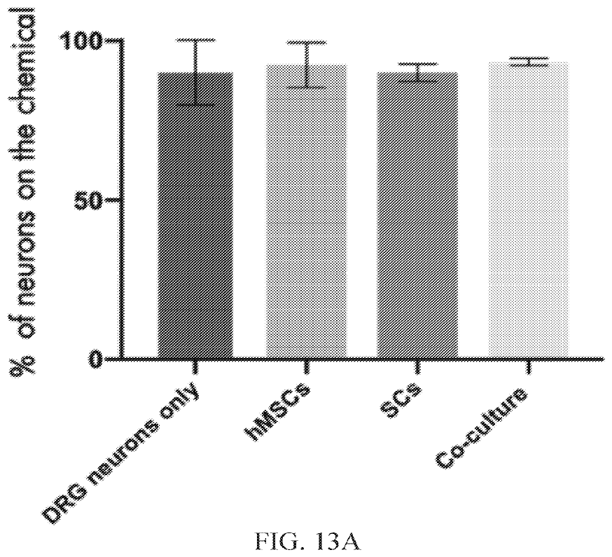

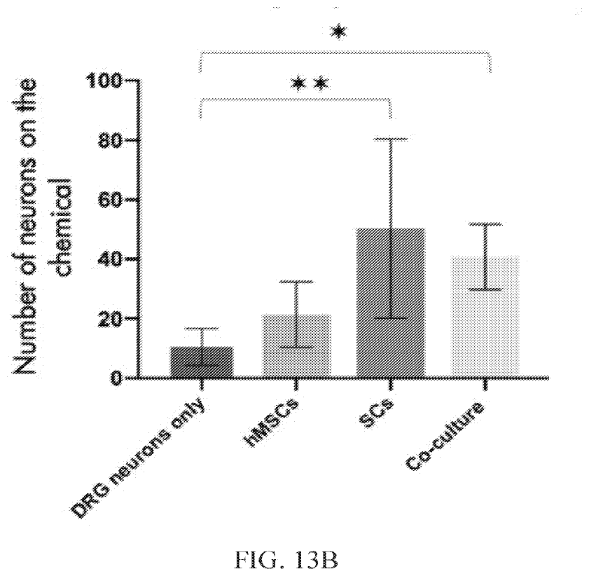

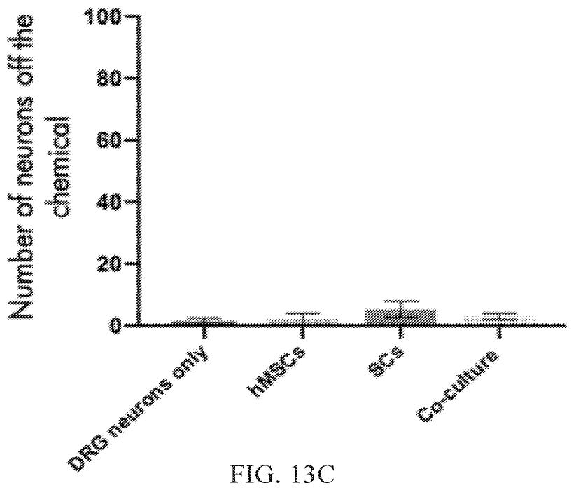

FIGS. 13A-13C show graphs indicating the results of various cell compositions cultured on chemically patterned OPF scaffolds. As shown, the OPF scaffolds promoted a high degree of DRG neuron attachment on the chemical. Error bars represent SEM. FIG. 13A shows equivalent high percentages of neurons on the chemical at different growth conditions. FIG. 13B shows that SC-loaded and co-cultured scaffolds promoted a significantly higher number of neurons on the chemical in comparison to scaffolds seeded with neurons only, as indicted by the p-values from the one-way ANOVA and Tukey's post-hoc test; * indicating p<0.05 and ** p<0.01. FIG. 13C shows equivalently low numbers of neurons off the chemical when compared with the different growth conditions.

### Neurite Density

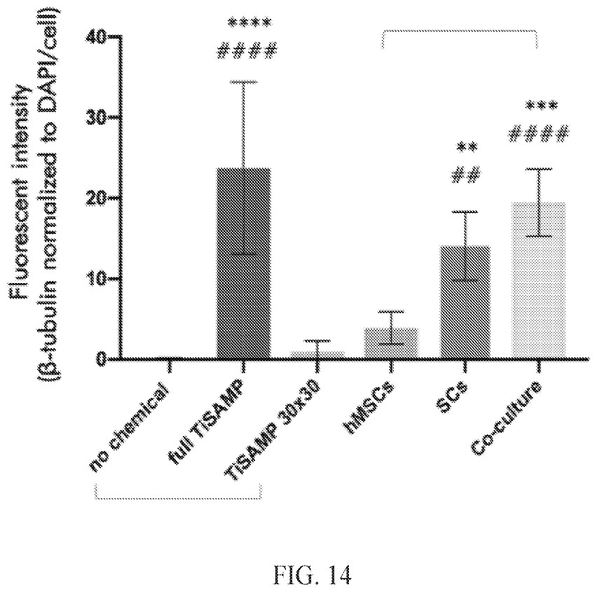

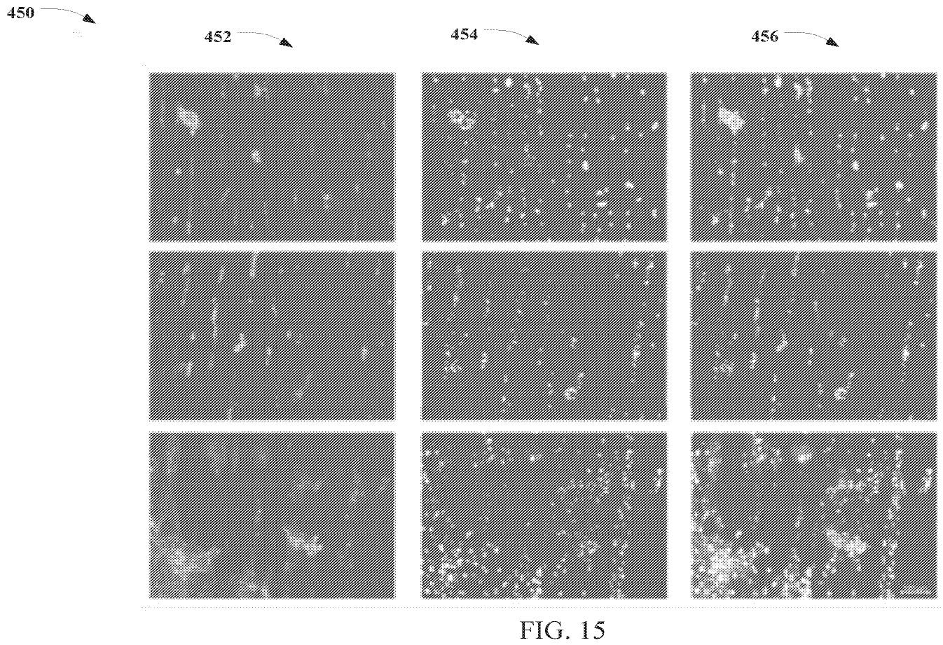

To assess the capacity of hMSC and SC co-cultured scaffolds to augment DRG neural extension, neurite density was compared between co-culture, single cell type and fibronectin covered scaffolds. Within the last group further distinction was made between scaffolds treated with 30 μm×30 μm TiO₂/SAMP chemical pattern (TiSAMP 30×30), unpatterned chemical (full TiSAMP) and sheets without chemical. Neurite density was quantitated by measuring β-tubulin fluorescent intensity normalized to DAPI staining intensity per cell (Table 3, FIG. 14).

Co-culturing hMSCs and SCs onto OPF sheets was found to support the second highest neurite density (19.47±2.07), with a significant increase compared to scaffolds seeded with hMSCs only (3.90±1.00) (p=0.03). A trend of co-cultures promoting higher neurite density in comparison to the SC group (14.03±2.12) was identified, albeit not a statistically significant one (p=0.62). Neurite density was determined to be highest on scaffolds that were fully covered with unpatterned chemistry (full TiSAMP) (23.72±5.32), suggesting that the surface chemistry is cell-adhesive in nature. However, neurons showed no spatial directionality, as displayed in FIG. 9. Significantly higher neurite density was found on SC- and co-cultured scaffolds compared to fibronectin coated sheets with TiSAMP 30×30 (0.92±0.53) (p=0.05 and p=0.004 respectively) and without chemical (0.10±0.07) (p=0.04 and p=0.004 respectively). These findings provide support for the idea above that loading OPF scaffolds with SCs or co-cultures increases neuronal attachment.

TABLE 3
β-tubulin fluorescent intensity measured as the mean grey value and normalized to DAPI
staining intensity per cell at different growth conditions. Data are displayed as mean ± SEM.

| | No | Full | TiSAMP | | | |
|---|---|---|---|---|---|---|
| | chemical | TiSAMP | 30 × 30 | hMSCs | SCs | Co-culture |
| | (n = 3) | (n = 4) | (n = 7) | (n = 4) | (n = 4) | (n = 4) |
| β-tubulin/ | 0.10 ± 0.07 | 23.72 ± 5.32 | 0.92 ± 0.53 | 3.90 ± 1.00 | 14.03 ± 2.12 | 19.47 ± 2.07 |
| (DAPI/cell) | | | | | | |

To assess the capacity of hMSC and SC co-cultured scaffolds to augment DRG neural extension, neurite density was compared between co-culture, single cell type and fibronectin covered scaffolds. Within the last group further distinction was made between scaffolds treated with 30 μm×30 μm TiO₂/SAMP chemical pattern (TiSAMP 30×30), unpatterned chemical (full TiSAMP) and sheets without chemical. Neurite density was quantitated by measuring β-tubulin fluorescent intensity normalized to DAPI staining intensity per cell (Table 3, FIG. 12).

FIG. 14 shows a graphical representation of β-tubulin fluorescent intensity in different growth condition groups. Error bars display SEM. Co-cultures supported significantly higher neurite density compared to hMSCs alone (& indicating p<0.05). Significant differences in neurite density were found between co-culture, SC and full TiSAMP groups to fibronectin coated OPF sheets with TiSAMP 30×30 and without chemical. # indicates comparison to fibronectin coated scaffolds patterned with TiSAMP 30×30, * indicates comparison to fibronectin coated sheets without chemical (##, ** p<0.01; *** p<0.001; ####, **** p<0.0001). No chemical, full TiSAMP and TiSAMP 30×30 were neurons only conditions. One-way ANOVA and Tukey's post-hoc test were run to compare experimental groups.

### The Effect of a hMSC and SC Co-Culture on ECM Deposition

The impact of co-culturing hMSCs and SCs on ECM deposition was evaluated by measuring fibronectin fluorescent intensity normalized to DAPI staining intensity per cell after 10 days of culture (Table 4). Co-cultures with DRG neurons promoted the highest fibronectin deposition (39.90±4.34) with a significant difference to both SCs (24.95±1.23) and hMSCs (15.00±3.80) with neurons (p=0.03 and 0.002 respectively). Interestingly, scaffolds seeded with SCs and neurons led to a significantly greater fibronectin deposition compared to scaffolds seeded with hMSCs and neurons (p=0.047). Notably, the assembled ECM fibrils exhibited a spatial directionality according to the chemical pattern and neurites were shown to weave through the fibrillar network of the naturally-deposited matrix.

FIG. 15 shows the spatially directed ECM fibrils of chemically micropatterned OPF scaffolds after 10 days of culture. These immunofluorescent images were captured at 10× magnification. Samples were stained for fibronectin (green) and DAPI (cell nuclei, white). Specifically, FIG. 15 shows a series of immunofluorescent images 450 with different staining targets and for various OPF sheets. All images within FIG. 15 were chemical patterned (e.g., a TiO₂/SAMP layer) having a 30 μm width repeating pattern (e.g., 30 μm×30 μm), as discussed above. Column 452 represents all images within FIG. 15 being stained for fibronectin, where the fibronectin appeared as green. Column 454 represents all images within FIG. 15 being stained for cell nuclei (e.g., using 4′,6-diamidino-2-phenylindole “DAPI”), where the cell nuclei appeared as white. Column 456 is the superimposed combination of the images of columns 452 and 454, for a given row such that a given image in column 456 is stained for both fibronectin and cell nuclei, which had appeared as containing green and white. The first row of images in FIG. 15 show scaffolds with human mesenchymal stromal cells, the second row of images in FIG. 15 show scaffolds with Schwann cells, and the third row of images in FIG. 15 show scaffolds with a co-culture of human mesenchymal stromal cells, and Schwann cells.

TABLE 4
Fibronectin fluorescent intensity measured as the mean grey value
and normalized to DAPI staining intensity per cell at different
growth conditions. Data are presented as mean ± SEM.

| | hMSCs | SCs | Co-culture |
|---|---|---|---|
| | (n = 4) | (n = 4) | (n = 3) |
| Fibronectin/ | 15.00 ± 3.80 | 24.95 ± 1.23 | 39.90 ± 4.34 |
| (DAPI/cell) | | | |

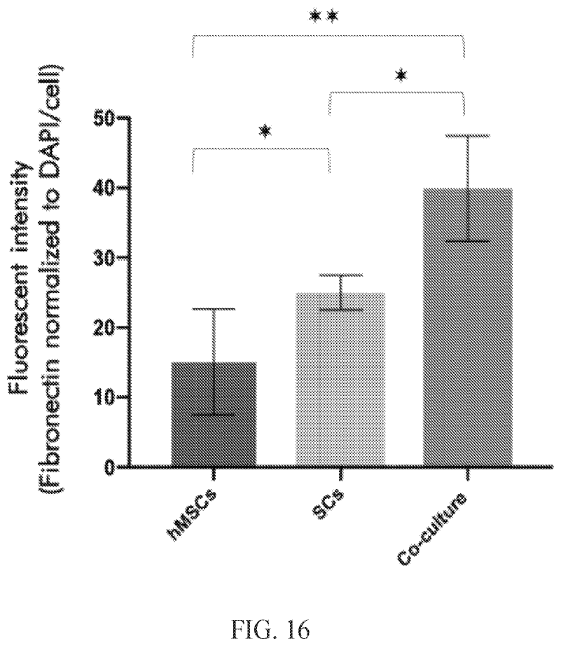

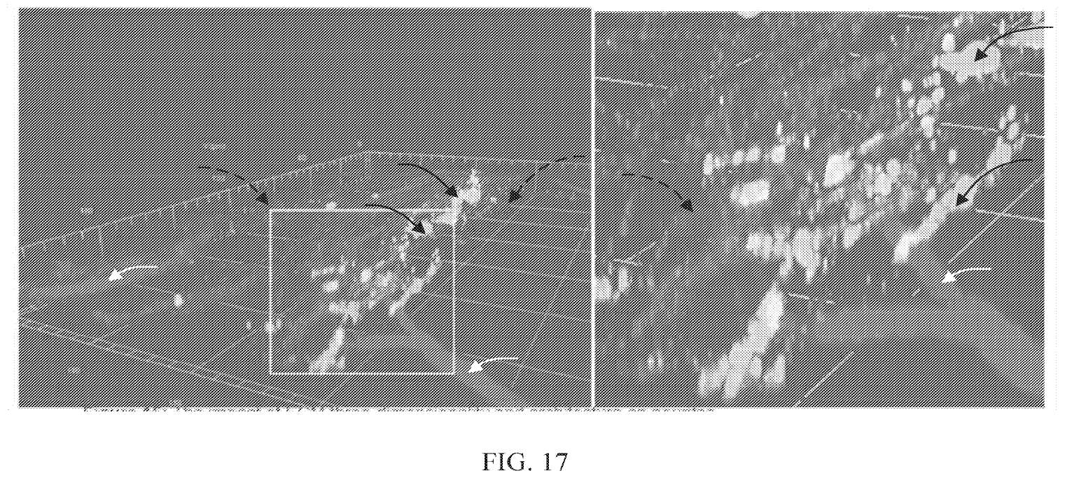

The impact of co-culturing hMSCs and SCs on ECM deposition was evaluated by measuring fibronectin fluorescent intensity normalized to DAPI staining intensity per cell after 10 days of culture (Table 4, FIG. 16). Co-cultures with DRG neurons promoted the highest fibronectin deposition (39.90±4.34) with a significant difference to both SCs (24.95±1.23) and hMSCs (15.00±3.80) with neurons (p=0.03 and 0.002 respectively). Interestingly, scaffolds seeded with SCs and neurons led to a significantly greater fibronectin deposition compared to scaffolds seeded with hMSCs and neurons (p=0.047). Notably, the assembled ECM fibrils exhibited a spatial directionality according to the chemical pattern (FIG. 15) and neurites were shown to weave through the fibrillar network of the naturally-deposited matrix (FIG. 17).

FIG. 16 shows a graphical representation of fibronectin fluorescent intensity in different growth condition groups. Error bars represent SEM. Co-cultures with DRG neurons resulted in the highest fibronectin deposition with a significant difference to hMSC and SC groups (* indicating p<0.05 and ** p<0.01). SCs and neurons significantly increased fibronectin deposition in comparison to the hMSC group. One-way ANOVA and Tukey's post-hoc test were used for group comparisons.

FIG. 17 shows the impact of ECM three-dimensionality and architecture on neurites. The left side of FIG. 17, shows a 3D reconstruction of a confocal z-stack image with a 50 microns scale bar. The right side of FIG. 17 is a zoomed-in section of the left side of FIG. 17. Samples were stained for fibronectin (green, black arrows in FIG. 17), β-tubulin (neurons, red, white arrows of FIG. 17) and DAPI (cell nuclei, blue, dashed arrows of FIG. 17) after Schwann cells and neurons had been in culture for 10 and 4 days, respectively. Neurites were observed to weave over and through the native, SC-specific ECM fibrils, demonstrating that the three-dimensional architecture influences the spreading of neurons across the polymer surface.

### Discussion

Without being bound by theory, in the present study, we hypothesized that co-culture of hMSCs and SCs on TiO₂/SAMP patterned OPF scaffolds supports neurite alignment, attachment and density. Our results show that the chemically micropatterned scaffolds promoted a high degree of neuronal attachment on and alignment along the bidirectional stripes regardless of cell type. However, scaffolds with SCs or co-cultured cells further enhanced neuronal attachment and led to a significantly higher neurite density compared to fibronectin coated scaffolds with or without the chemical pattern. In addition, co-cultures significantly increased neurite density compared to hMSCs alone. By studying the cell-assembled ECM, we found that co-cultures with dissociated DRG neurons led to significantly greater fibronectin deposition compared to each cell type alone. Interestingly, SCs with neurons supported a significantly higher fibronectin deposition than hMSCs with neurons.

### Methodology Discussion

A variety of synthetic biomaterials have been investigated for their use in SCI. In the present study, OPF was employed as the substrate for our new spiral scaffold design, harnessing the macroporous structure of hydrogels, which poses an effective medium for neuroregeneration [Refs. 81, 85]. Notably, OPF was demonstrated to most closely resemble rat spinal cord in terms of its mechanical properties (3-point bending and stiffness), when compared to PLGA and PCLF [Ref. 5]. Its compatibility within native cord tissue may improve scaffold integration and survival once implanted into transection lesions. In the past, the Windebank laboratory has demonstrated that charge modification of the OPF surface via MAETAC copolymerization, yielding OPF+, improved neuron attachment leading to significantly greater neurite outgrowth in vitro [Ref. 93] and axonal regeneration in vivo [Ref. 5] when compared to neutral OPF. However, this was the first attempt to integrate the moisture-sensitive chemicals titanium dioxide and phosphonic acid into highly aqueous OPF hydrogel, which involved, unsurprisingly, some troubleshooting. Additively, the charges of OPF+ were likely to interfere with the charges of the utilized chemicals. Taken together, the rationale was to first optimize chemical patterning on OPF and to use OPF+ as a substrate in the future.

The macro-architecture of scaffolds has been revealed to be of great importance in terms of axonal regeneration and environmental modulation [Ref 95]. Our new scaffold design is an OPF hydrogel sheet that spontaneously spirals up when exposed to liquid, thereby approximating the structure of the native spinal cord. A core advantage of the design is the maximization of available surface area for regenerating axons due to increased open space. This might provide a matrix for axons to attach to since it was observed that the axons would elongate along multichannel scaffolds' exterior surface [Refs. 14, 95]. Moreover, the sheet is only 80 μm thin, thereby facilitating diffusion of nutrients, oxygen, and waste products.

Numerous aligned micro-topographies have been investigated in an attempt to spatially guide regenerating neurites and/or axons [Refs. 96-98, 102, 103]. The present work aimed at neurite alignment with a cell-adhesive chemical patterning approach utilizing titanium and phosphonic acid. This surface chemistry was shown to yield two-dimensional, ultrastructural cellular alignment [Ref 6] and was recently demonstrated to form three-dimensional fibrillar networks of a fibroblast-derived ECM in accordance to the pattern [Ref. 7]. A major strength of our method is that stripes can be generated that are only microns apart, which is closer than what is able to be physically molded. Additionally, with the SAMP-TiO₂ surface structures ranging between 10 nm-70 nm, the pattern does not alter the fundamental structure of the polymer nor its physical properties [Ref 6]. This nanometer scale also allows cells to spread from generated stripes across chemistry-free intervals, yet maintaining alignment with the pattern to form a spatially organized confluent monolayer [Ref 6].

The photolithographic and chemical vapor deposition-based sequence applied so far needed to be modified in order to fabricate the pattern on a OPF hydrogel surface. Instead of TiO₂/SAMP being prepared from a pattern that was spin-cast with HMDS and AZ-5412E photoresist, [Refs. 6, 7] manufactured shadow-masks were directly placed onto the sheets. Subsequent exposure to titanium, heat and phosphonic acid (as specified in 5.1.3) created the two-component chemical interface. A major challenge faced was to achieve homogenous chemical pattern across the entire scaffold surface, which was hampered by multiple factors. Fraying of and chemical residue on Kapton shadow masks, for instance, could cause distortion of the stripes. On that account, work was done to obtain masks by reproducible and expeditious means in collaboration with the Mayo Clinic Engineering Department. Additionally, the 200 g weight needed to be evenly applied onto the mask-scaffold composite, otherwise the debossing would not be uniform. By working with different weights, it was shown that lighter ones yielded no pattern and heavier ones would press the mask down too much making it melt into the polymer. Utilized chemicals themselves posed another factor, their moisture and oxygen-sensitive property rendering them amenable to manipulation. This was counteracted by handling them strictly inside the glove bag, by getting the OPF surface as dry as possible prior to debossing, and by performing washes with toluene. These factors did not, however, interfere with study outcomes, since each chemically patterned sample was controlled before its experimental inclusion to ensure adequate quality and flawed samples were excluded. Nevertheless, the OPF chemical patterning process remains to be further optimized in order to obtain more homogenous samples.

PEG-based hydrogels without additional microstructural or charge modification are known to have low cell attachment rates [Ref 93]. This was overcome firstly by the cell-adhesive property of the chemical interface itself, [Refs. 139, 140] and secondly by covering scaffolds with fibronectin protein coats or seeding them with different cell types. Multiple neurite outgrowth studies have demonstrated the cell-attractive and axonal growth-supportive attributes of fibronectin, making it an efficient coating substrate [Ref 90]. Nevertheless, some detachment-issues on scaffolds seeded only with neurons were encountered, rendering further optimization necessary. Using a decellularized ECM may be a prospective attempt in achieving superior neural cell attachment, since it provides several proteins, including fibronectin, laminin and collagen, and multiple anchoring points due to its three-dimensionality [Ref. 114]. Once dissociated DRG neurons were co-cultured with hMSCs and SCs, the OPF sheets were not coated with fibronectin, since these cells are proposed to synthesize fibronectin along with other extracellular matrix molecules on their own [Refs. 121, 122, 128]. In this way, the analyzed effect on neurites is solely attributable to the environment established by the cells themselves.

As to ECM deposition by hMSCs and SCs, this work focused on fibronectin due to its reparative functions in tissue damage and capacity to sequester cell-secreted growth factors [Refs. 108, 110]. It would be also of great interest to study the impact of co-culturing hMSCs and SCs on the deposition of laminin, due to its innate regenerative role in the healthy CNS, and collagen, which provides structural support and regenerative potential [Refs. 99, 108, 130]. However, due to the time limitation, this could not be carried out during the duration of this project. Additionally, in the future, it may be interesting to investigate the cell-specific growth factors, cytokines and exosomes sequestered by the co-cultured ECM which may also enhance neuroregeneration.

The soft property of OPF that makes it, amongst others, highly suitable for the injured spinal cord, also posed some difficulties: If not handled with outmost care, OPF sheets tore easily. Due to disruption of OPF sheets during the multiple steps of handling, low attachment rates, and time-constraints, the ‘n value’ in the experiments is low. Ideally, the experiments would be repeated so as to increase the statistical power of the study.

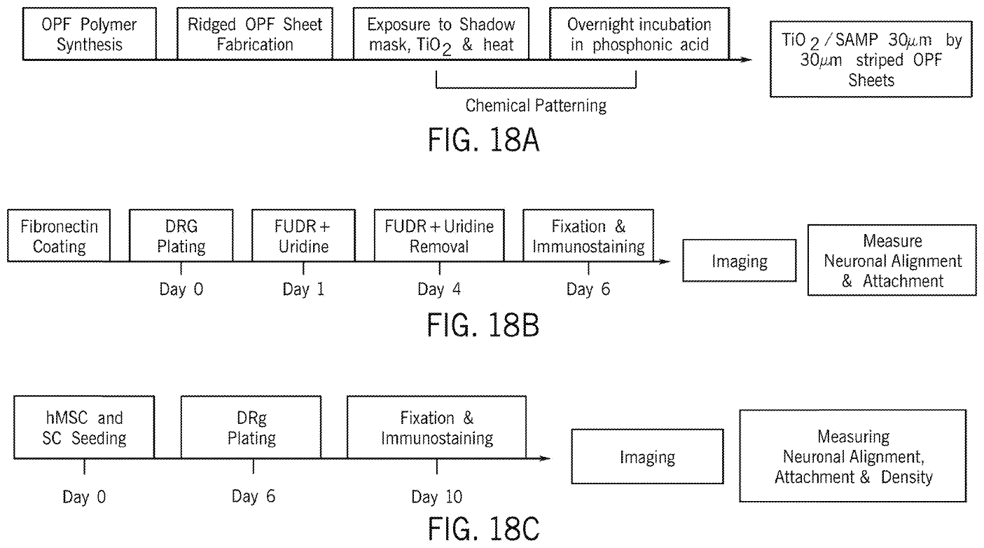

FIGS. 18A-18C show various processes for creating the OPF sheets with the chemical patterning, seeding cells on the sheets, imaging the sheets with the cells, and measuring the cells.

### Discussion of Results and Literature

Neuroanatomical and functional recovery after SCI requires appropriate topographical cues for axons to re-establish neuronal pathways. Our approach for promoting neurite alignment utilized a two-component, micropatterned surface chemistry. The chemical template is able to control the orientation of cells and their self-assembled ECM fibrils along its pattern on OPF hydrogel sheets, as demonstrated by the present study, and across the surface of various other polymers [Refs. 6, 7]. Moreover, we were able to show that cells predominantly attached on the stripes and that scaffolds entirely covered with TiO₂/SAMP supported the highest neurite density. Both findings indicate that the surface chemistry has cell-adhesive properties, an observation that is congruent with other studies' findings, where the nanoscale interface was shown to augment cell affinity for various biopolymer surfaces [Refs. 139, 140].

No single therapy will be sufficient to achieve neurological recovery after SCI; we therefore investigated the combination of SCs and hMSCs on chemically patterned OPF and assessed their efficiency in enhancing neuronal attachment and extension. Numerous studies have demonstrated the neuro-regenerative capacity of SCs, particularly in combination with biomaterial scaffolds [Refs. 11, 14, 78]. Apart from their capability to (re)myelinate axons, the cells are proposed to enhance axon guidance and re-growth by expressing adhesion molecules on their surface and by synthesizing a number of growth factors [Refs. 119, 120]. However, to date, the potential of SCs co-cultured with another cell type in increasing axonal regeneration has hardly been studied and has become an object of research only within the last few years. In the PNS, research on rat sciatic nerve regeneration has shown that SCs co-cultured with adipose-derived adult stem cells (“ASCs”) on therapeutic nerve conduits led to greater functional recovery than single cell type-conduits [Ref. 141]. In vitro studies confirmed concordantly that co-culture of these cell types upregulated the expression of neurotrophic factors [Ref. 141]. Accordingly, a recent study demonstrated that bone marrow stromal cells (“BMSCs”) stimulated the proliferation of SCs in a co-culture system without intercellular contact [Ref 142]. This was suggested to be due to bioactive molecules and ECM proteins secreted by BMSCs, which might have exerted a stimulatory effect on SCs. Notably, in rat models of SCI, SCs co-transplanted with BMSCs into the lesion's epicenter, either on multichannel PLGA scaffolds [Ref. 143] or by themselves, [Ref. 144] promoted enhanced axonal regeneration and functional recovery compared to single cell type-grafts. To our best knowledge, this is the first work so far that studies the effect of co-culturing hMSCs and SCs on neurite regeneration on OPF scaffolds. Our results demonstrate that SCs led to the highest and co-cultures to the second highest number of neurons on the chemical. Interestingly, co-cultured scaffolds, however, promoted a higher neurite density than SC cultured ones. In addition, co-cultures led to a significant increase in neurite density compared to hMSCs alone. Based on these promising findings the assumption can be made that the hybrid environment vested with bioactive molecules of both cell types might stimulate neurite extension to a greater extent than a single cell type-environment. Similar to the findings by the investigators cited above, [Refs. 141, 142] hMSCs and SCs may interact synergistically to increase their regenerative capabilities and the expression of growth factors and other cytokines might be upregulated by their co-culture. However, future work is needed to further study the reciprocal effects hMSCs and SCs exert on each other and the environment co-established by these cells to find a more definite explanation as to how neurite density is enhanced.

The present study further found that scaffolds seeded with SCs or a mixed population of SCs and hMSCs significantly increased neurite density compared to fibronectin coated scaffolds with or without the chemical pattern. Purified ECM proteins, including fibronectin, laminin and collagen, have been consistently utilized for two-dimensional surface coating to enhance axonal adhesion and growth [Refs. 7, 96, 98, 114]. However, this approach lacks in mimicking the complexity of the native ECM. Using a naturally deposited, cell-derived ECM contributes a more complete environment with numerous proteins, neurotrophic factors and a three-dimensional structure providing multiple contact points [Ref 114]. It seems likely that this multifaceted and complex environment provides greater neuro-regenerative potential than a single ECM protein in two dimensions. In our study, SCs and hMSCs have contributed aligned, self-assembled ECM fibrils (as shown by FIGS. 14 and 15) that may facilitate neurite extension and therefore increase neurite density. This idea is supported by a recent study by Harris et al. which found that a decellularized, fibroblast-derived ECM deposited on PET polymer promoted significantly longer neurite extension than the polymeric surface coated with fibronectin [Ref 7]. Considering that fibronectin was the main protein in the decellularized matrix, it was suggested that neuron-fibronectin interactions might be modulated by the ECM composition. Additionally, aligned ECM fibrils provided a pathway for growth cone motility that was proposed to facilitate neurite elongation [Ref. 7]. In the present work, it would, however, require further assessment as to whether the observed effects are due to the deposited ECM, the cells themselves or a combination of both. This could be partly achieved by decellularizing the cell-deposited ECM and study the effect of the remaining matrix on neurites. The future direction for this project therefore involves decellularizing the SC- and hMSC-assembled hybrid ECM on the patterned OPF scaffolds, by previously described methods [Ref. 113].

Therapies involving natively assembled ECM are an emerging field in SCI research, aiming at a regenerative environment with structural organization [Refs. 7, 113,114]. Notably, recent studies in our laboratory showed for the first time that co-culturing hMSCs and SCs on OPF scaffolds without neurons for 7 days led to a 5-fold increase in fibronectin deposition compared to each cell type alone. The current project further demonstrated that co-culture of these cells together with dissociated DRG neurons significantly enhanced ECM deposition after 10 days of culture in comparison to single cell type-scaffolds. Previous studies have similarly demonstrated the value of combining different cell types in terms of ECM synthetization. Work performed by Levorson et al [Ref 145] and Bian et al. [Ref. 146] on polymer/ECM hybrid constructs showed that co-culturing MSCs and chondrocytes led to increased matrix production. It was suggested that MSCs exert a stimulatory effect on chondrocytes through secreted growth factors and direct cell-cell contact. Accordingly, in our study, hMSCs and SCs might reciprocally support their fibronectin production, possibly through the secretion of growth factors. It should be taken into account that neurons and neuronal supporting cells themselves are known to synthesize ECM proteins, [Ref. 149] and that their contribution to the ECM deposition could not be distinguished in our current experiments. In the future, this could be solved by using antibodies specific to human or rat fibronectin, as previously described [Ref 7].

Interestingly, SCs seeded onto OPF scaffolds with dissociated DRG neurons were demonstrated to deposit a significantly greater amount of fibronectin than hMSCs and neurons after 10 days of culture. The present finding is contrary to recent observations in our laboratory, where SCs cultured alone onto OPF sheets produced significantly less fibronectin than hMSCs after one week. Moreover, it is generally assumed, that hMSCs secrete abundant amounts of ECM proteins, while SCs lay down a limited amount of ECM when cultured alone [Refs. 12, 128]. As the present result differs from other studies, possible explanations should be discussed. It has become increasingly clear that a direct physical contact between SCs and axons promotes juxtacrine signals that are critical for SC fate [Refs. 147, 148]. Mechanical stimuli from axo-glial interactions and their transduction into biological responses are not only necessary for myelination but also for the deposition of ECM by SCs [Ref. 148]. Previous experiments in our laboratory on ECM deposition were conducted with SCs alone, whereas in the present study SCs were co-cultured with dissociated neurons from rat DRG. Physical contact between neurites and SCs triggering increased ECM synthetization might be a possible underlying reason for the present finding.

### Future Directions

The re-creation of an ECM that provides residing cells with biomechanical regulatory and signaling cues and structural support is critical after SCI. We aimed at generating a hybrid matrix deposited by a SC and hMSC co-culture on OPF scaffolds in alignment with a 30 μm×30 μm TiO₂/SAMP patterned interface.

The next step has been to generate micro-topographical stripes that were only 20 μm or possibly 10 μm apart in order to obtain an aligned, cell-assembled ECM that is more densely configured and amenable to decellularization. Once decellularized, the challenge will be to roll the ECM/OPF hybrid constructs up into a three-dimensional, spiral configuration without disturbing the architecture of the matrix. We have already started attempts to convolve chemically patterned OPF sheets with a fibroblast-derived ECM that had been decellularized by our co-inventors at Princeton University. Captured immunofluorescent images demonstrated that the ultrastructurally aligned ECM fibrils were successfully preserved across the scaffold's surface. Currently, our lab is working on achieving the same result with a decellularized ECM deposited by a hMSC and SC co-culture on patterned OPF.

The long-term direction of this project will be to transplant the chemically patterned OPF scaffolds, complemented with a decellularized hybrid ECM and loaded with SCs, into rat models of SCI to determine their efficiency in promoting neuroanatomical and functional recovery. Lastly, to create a product more clinically relevant, future studies will explore the use of human SCs, MSCs, and neurons in making a rich 3D ECM on these OPF scaffolds.

### Conclusions

We have fabricated TiO₂/SAMP chemically patterned OPF hydrogel sheets that promoted a high degree of neurite attachment on the chemical and alignment along the stripes across the polymer, the surface chemistry providing cell-adhesive, micro-topographical cues. By loading these scaffolds with SCs and hMSCs we demonstrated that neuronal attachment and density can be enhanced by co-culture of these cells or SCs alone. In addition, we found that hMSCs and SCs assembled their ECM fibrils spatially oriented by the patterned stripes and that their co-culture increased ECM deposition.

SCs and hMSCs may interact synergistically to augment their neuro-regenerative potential and create a hybrid environment that is vested with native bioactive molecules and supports neurite elongation. We are optimizing conditions for axonal regeneration after SCI in terms of topographical cues for cells and a growth-enhancing, cell-specific, three-dimensional hybrid matrix. Future studies with more statistical power and further analyzation of the environment co-established by hMSCs and SCs will be required. Prospectively, our ECM/OPF hybrid scaffolds will be investigated in animal models of SCI. Effective treatments for SCI are lacking and our new therapeutic approach may improve on previous strategies by incorporating the use of biomaterials, patterned cell-attractive surface chemistry, different cell types, and a natively assembled ECM.

### REFERENCES FOR EXAMPLE 1

[1] Spinal cord injury (Fact sheet No 384). In. www.who.int/news-room/fact-sheets/detail/spinal-cord-injury ed: World Health Organization; 2013.
[2] Anderson P N, Lieberman A R. Intrinsic Determinants of Differential Axonal Regeneration by Adult Mammalian CNS Neurons. In. Amsterdam: Harwood Academic Publishers; 1999.
[3] Ahad M. Siddiqui M K, Michael G. Fehlings. Translating mechanisms of neuroprotection, regeneration, and repair to treatment of spinal cord injury. In: Numa Dancause S N, Serge Rossignol, ed. Progress in brain research, Sensorimotor Rehabilitation. Vol 218.2015:15-54.
[4] Chen B K, Knight A M, de Ruiter G C, et al. Axon regeneration through scaffold into distal spinal cord after transection. J Neurotrauma. 2009; 26(10):1759-1771.
[5] Chen B K, Knight A M, Madigan N N, et al. Comparison of polymer scaffolds in rat spinal cord: a step toward quantitative assessment of combinatorial approaches to spinal cord repair. Biomaterials. 2011; 32(32):8077-8086.
[6] Donnelly P E, Jones C M, Bandini S B, Singh S, Schwartz J, Schwarzbauer J E. A Simple Nanoscale Interface Directs Alignment of a Confluent Cell Layer on Oxide and Polymer Surfaces. J Mater Chem B. 2013; 1(29):3553-3561.
[7] Harris G M, Madigan N N, Lancaster K Z, et al. Nerve Guidance by a Decellularized Fibroblast Extracellular Matrix. Matrix Biol. 2017; 60-61:176-189.
[8] Boido M, Garbossa D, Fontanella M, Ducati A, Vercelli A. Mesenchymal stem cell transplantation reduces glial cyst and improves functional outcome after spinal cord compression. World Neurosurg. 2014; 81(1):183-190.
[9] Oliveri R S, Bello S, Biering-Sorensen F. Mesenchymal stem cells improve locomotor recovery in traumatic spinal cord injury: systematic review with meta-analyses of rat models. Neurobiol Dis. 2014; 62:338-353.
[10] Takami T, Oudega M, Bates M, et al. Schwann cell but not olfactory ensheathing glia transplants improve hindlimb locomotor performance in the moderately contused adult rat thoracic spinal cord. The Journal of Neuroscience: The Official Journal of the Society for Neuroscience. 2002.
[11] Madigan N N, Chen B K, Knight A M, et al. Comparison of cellular architecture, axonal growth, and blood vessel formation through cell-loaded polymer scaffolds in the transected rat spinal cord. Tissue Eng Part A. 2014; 20(21-22):2985-2997.
[12] Su Y, Denbeigh J M, Camilleri E T, et al. Extracellular matrix protein production in human adipose-derived mesenchymal stem cells on three-dimensional polycaprolactone (PCL) scaffolds responds to GDF5 or FGF2. Gene Rep. 2018; 10:149-156.
[13] Isaacman-Beck J, Schneider V, Franzini-Armstrong C, Granato M. The lh3 Glycosyltransferase Directs Target-Selective Peripheral Nerve Regeneration. Neuron. 2015; 88(4):691-703.
[14] Chen B K, Madigan N N, Hakim J S, et al. GDNF Schwann cells in hydrogel scaffolds promote regional axon regeneration, remyelination and functional improvement after spinal cord transection in rats. J Tissue Eng Regen Med. 2018; 12(1):e398-e407.
[15] Albin M, Shoemaker W, Thompson W, Holbrook P. Spinal Cord Injury Facts and Figures at a Glance. In. Available at: www.nscisc.uab.edu/public_pages/FactsFiguresArchives/SCI%20Facts%20and%20Figures%20at%20a%20Glance%202017.pdf ed. Philadelphia: National Spinal Cord Injury Statistical Center; 2017:928-932.
[16] Ge L, Arul K, Ikpeze T, Baldwin A, Nickels J L, Mesfin A. Traumatic and Nontraumatic Spinal Cord Injuries. World Neurosurg. 2018; 111:e142-e148.
[17] Fitzharris M, Cripps R A, Lee B B. Estimating the global incidence of traumatic spinal cord injury. Spinal Cord. 2014; 52(2):117-122.
[18] Devivo M J. Epidemiology of traumatic spinal cord injury: trends and future implications. Spinal Cord. 2012; 50(5):365-372.
[19] van den Berg M E, Castellote J M, Mahillo-Fernandez I, de Pedro-Cuesta J. Incidence of spinal cord injury worldwide: a systematic review. Neuroepidemiology. 2010; 34(3):184-192; discussion 192.
[20] Waters R L, Adkins R H, Yakura J S. Definition of complete spinal cord injury. Paraplegia. 1991; 29(9):573-581.
[21] Kirshblum S C, Burns S P, Biering-Sorensen F, et al. International standards for neurological classification of spinal cord injury (revised 2011). J Spinal Cord Med. 2011; 34(6):535-546.
[22] DeVivo M J. Epidemiology of Spinal Cord Injury. Spinal Cord Medicine Principles and Practice. 2010:78-84.
[23] Madigan N N, McMahon S, O'Brien T, Yaszemski M J, Windebank A J. Current tissue engineering and novel therapeutic approaches to axonal regeneration following spinal cord injury using polymer scaffolds. Respir Physiol Neurobiol. 2009; 169(2):183-199.
[24] Sezer N, Akkus S, Ugurlu F G. Chronic complications of spinal cord injury. World J Orthop. 2015; 6(1):24-33.
[25] Chierzi S, Ratto G M, Verma P, Fawcett J W. The ability of axons to regenerate their growth cones depends on axonal type and age, and is regulated by calcium, cAMP and ERK. Eur J Neurosci. 2005; 21(8):2051-2062.
[26] Kwon B K, Liu J, Messerer C, et al. Survival and regeneration of rubrospinal neurons 1 year after spinal cord injury. Proc Natl Acad Sci USA. 2002; 99(5):3246-3251.
[27] Fawcett J W, Keynes R J. Peripheral nerve regeneration. Annu Rev Neurosci. 1990; 13:43-60. [28] McDonald J W, Sadowsky C. Spinal-cord injury. Lancet. 2002; 359(9304):417-425.
[29] Dumont R J, Okonkwo D O, Verma S, et al. Acute spinal cord injury, part I: pathophysiologic mechanisms. Clin Neuropharmacol. 2001; 24(5):254-264.
[30] Gerardo-Nava J, Mayorenko, I I, Grehl T, Steinbusch H W, Weis J, Brook G A. Differential pattern of neuroprotection in lumbar, cervical and thoracic spinal cord segments in an organotypic rat model of glutamate-induced excitotoxicity. J Chem Neuroanat. 2013; 53:11-17.
[31] Park E, Velumian A A, Fehlings M G. The role of excitotoxicity in secondary mechanisms of spinal cord injury: a review with an emphasis on the implications for white matter degeneration. J Neurotrauma. 2004; 21(6):754-774.
[32] Vosler P S, Sun D, Wang S, et al. Calcium dysregulation induces apoptosis-inducing factor release: cross-talk between PARP-1- and calpain-signaling pathways. Exp Neurol. 2009; 218(2):213-220.
[33] Bao F, Liu D. Peroxynitrite generated in the rat spinal cord induces apoptotic cell death and activates caspase-3. Neuroscience. 2003; 116(1):59-70.
[34] Chatzipanteli K, Garcia R, Marcillo A E, Loor K E, Kraydieh S, Dietrich W D. Temporal and segmental distribution of constitutive and inducible nitric oxide synthases after traumatic spinal cord injury: effect of aminoguanidine treatment. J Neurotrauma. 2002; 19(5):639-651.
[35] Sakamoto A, Ohnishi S T, Ohnishi T, Ogawa R. Relationship between free radical production and lipid peroxidation during ischemia-reperfusion injury in the rat brain. Brain Res. 1991; 554(1-2):186-192.
[36] Casha S, Yu W R, Fehlings M G. Oligodendroglial apoptosis occurs along degenerating axons and is associated with FAS and p75 expression following spinal cord injury in the rat. Neuroscience. 2001; 103(1):203-218.
[37] Haase G, Pettmann B, Raoul C, Henderson C E. Signaling by death receptors in the nervous system. Curr Opin Neurobiol. 2008; 18(3):284-291.
[38] Stence N, Waite M, Dailey M E. Dynamics of microglial activation: a confocal time-lapse analysis in hippocampal slices. Glia. 2001; 33(3):256-266.
[39] Bartholdi D, Schwab M E. Expression of pro-inflammatory cytokine and chemokine mRNA upon experimental spinal cord injury in mouse: an in situ hybridization study. Eur J Neurosci. 1997; 9(7):1422-1438.
[40] Gadani S P, Walsh J T, Lukens J R, Kipnis J. Dealing with Danger in the CNS: The Response of the Immune System to Injury. Neuron. 2015; 87(1):47-62.
[41] Schnell L, Fearn S, Schwab M E, Perry V H, Anthony D C. Cytokine-induced acute inflammation in the brain and spinal cord. J Neuropathol Exp Neurol. 1999; 58(3):245-254.
[42] Schachtrup C, Ryu J K, Helmrick M J, et al. Fibrinogen triggers astrocyte scar formation by promoting the availability of active TGF-beta after vascular damage. J Neurosci. 2010; 30(17):5843-5854.
[43] Herrmann J E, Imura T, Song B, et al. STAT3 is a critical regulator of astrogliosis and scar formation after spinal cord injury. J Neurosci. 2008; 28(28):7231-7243.
[44] Tang X, Davies J E, Davies S J. Changes in distribution, cell associations, and protein expression levels of NG2, neurocan, phosphacan, brevican, versican V2, and tenascin-C during acute to chronic maturation of spinal cord scar tissue. J Neurosci Res. 2003; 71(3):427-444.
[45] McKeon R J, Schreiber R C, Rudge J S, Silver J. Reduction of neurite outgrowth in a model of glial scarring following CNS injury is correlated with the expression of inhibitory molecules on reactive astrocytes. J Neurosci. 1991; 11(11):3398-3411.
[46] McKeon R J, Hoke A, Silver J. Injury-induced proteoglycans inhibit the potential for laminin-mediated axon growth on astrocytic scars. Exp Neurol. 1995; 136(1):32-43.
[47] Dyck S M, Alizadeh A, Santhosh K T, Proulx E H, Wu C L, Karimi-Abdolrezaee S. Chondroitin Sulfate Proteoglycans Negatively Modulate Spinal Cord Neural Precursor Cells by Signaling Through LAR and RPTPsigma and Modulation of the Rho/ROCK Pathway. Stem Cells. 2015; 33(8):2550-2563.
[48] Monnier P P, Sierra A, Schwab J M, Henke-Fahle S, Mueller B K. The Rho/ROCK pathway mediates neurite growth-inhibitory activity associated with the chondroitin sulfate proteoglycans of the CNS glial scar. Mol Cell Neurosci. 2003; 22(3):319-330.
[49]. Koprivica V, Cho K S, Park J B, et al. EGFR activation mediates inhibition of axon regeneration by myelin and chondroitin sulfate proteoglycans. Science. 2005; 310(5745):106-110.
[50] Wanner I B, Anderson M A, Song B, et al. Glial scar borders are formed by newly proliferated, elongated astrocytes that interact to corral inflammatory and fibrotic cells via STAT3-dependent mechanisms after spinal cord injury. J Neurosci. 2013; 33(31):12870-12886.
[51] Hu R, Zhou J, Luo C, et al. Glial scar and neuroregeneration: histological, functional, and magnetic resonance imaging analysis in chronic spinal cord injury. J Neurosurg Spine. 2010; 13(2):169-180.
[52] Ramon y Cajal S. Degeneration and regeneration of the nervous system. In. London, Oxford: Univ. Press1928.
[53] Logan A, Green J, Hunter A, Jackson R, Berry M. Inhibition of glial scarring in the injured rat brain by a recombinant human monoclonal antibody to transforming growth factor-beta2. Eur J Neurosci. 1999; 11(7):2367-2374.
[54] Soderblom C, Luo X, Blumenthal E, et al. Perivascular fibroblasts form the fibrotic scar after contusive spinal cord injury. J Neurosci. 2013; 33(34):13882-13887.
[55] Goritz C, Dias D O, Tomilin N, Barbacid M, Shupliakov O, Frisen J. A pericyte origin of spinal cord scar tissue. Science. 2011; 333(6039):238-242.
[56] Cregg J M, DePaul M A, Filous A R, Lang B T, Tran A, Silver J. Functional regeneration beyond the glial scar. Exp Neurol. 2014; 253:197-207.
[57] Chen M S, Huber A B, van der Haar M E, et al. Nogo-A is a myelin-associated neurite outgrowth inhibitor and an antigen for monoclonal antibody IN-1. Nature. 2000; 403(6768):434-439.
[58] Chopp M, Zhang X H, Li Y, et al. Spinal cord injury in rat: treatment with bone marrow stromal cell transplantation. Neuroreport. 2000; 11(13):3001-3005.
[59] Kottis V, Thibault P, Mikol D, et al. Oligodendrocyte-myelin glycoprotein (OMgp) is an inhibitor of neurite outgrowth. J Neurochem. 2002; 82(6):1566-1569.
[60] Moreau-Fauvarque C, Kumanogoh A, Camand E, et al. The transmembrane semaphorin Sema4D/CD100, an inhibitor of axonal growth, is expressed on oligodendrocytes and upregulated after CNS lesion. J Neurosci. 2003; 23(27):9229-9239.
[61] Park J B, Yiu G, Kaneko S, et al. A TNF receptor family member, TROY, is a coreceptor with Nogo receptor in mediating the inhibitory activity of myelin inhibitors. Neuron. 2005; 45(3):345-351.
[62] Wong S T, Henley J R, Kanning K C, Huang K H, Bothwell M, Poo M M. A p75(NTR) and Nogo receptor complex mediates repulsive signaling by myelin-associated glycoprotein. Nat Neurosci. 2002; 5(12):1302-1308.
[63] Mi S, Lee X, Shao Z, et al. LINGO-1 is a component of the Nogo-66 receptor/p75 signaling complex. Nat Neurosci. 2004; 7(3):221-228.
[64] McKerracher L, Higuchi H. Targeting Rho to stimulate repair after spinal cord injury. J Neurotrauma. 2006; 23(3-4):309-317.
[65] Rowland J W, Hawryluk G W, Kwon B, Fehlings M G. Current status of acute spinal cord injury pathophysiology and emerging therapies: promise on the horizon. Neurosurg Focus. 2008; 25(5):E2.
[66] Bracken M B. Steroids for acute spinal cord injury. Cochrane Database Syst Rev. 2012; 1:Cd001046.
[67] Hurlbert R J. Methylprednisolone for the treatment of acute spinal cord injury: point. Neurosurgery. 2014; 61 Suppl 1:32-35.
[68] Royer B, Lee K, Gruson B, et al. Methylprednisolone-induced immune thrombocytopenia. In: Blood. Vol 115. United States 2010:5431-5432.
[69] Silva N A, Sousa N, Reis R L, Salgado A J. From basics to clinical: a comprehensive review on spinal cord injury. Prog Neurobiol. 2014; 114:25-57.
[70] Cengiz S L, Kalkan E, Bayir A, Ilik K, Basefer A. Timing of thoracolumbar spine stabilization in trauma patients; impact on neurological outcome and clinical course. A real prospective (rct) randomized controlled study. Arch Orthop Trauma Surg. 2008; 128(9):959-966.
[71] Fehlings M G, Perrin R G. The timing of surgical intervention in the treatment of spinal cord injury: a systematic review of recent clinical evidence. Spine (Phila Pa. 1976). 2006; 31(11 Suppl):S28-35; discussion S36.
[72] Liu S, Aghakhani N, Boisset N, Said G, Tadie M. Innervation of the caudal denervated ventral roots and their target muscles by the rostral spinal motoneurons after implanting a nerve autograft in spinal cord-injured adult marmosets. J Neurosurg. 2001; 94(1 Suppl):82-90.
[73] Tadie M, Liu S, Robert R, et al. Partial return of motor function in paralyzed legs after surgical bypass of the lesion site by nerve autografts three years after spinal cord injury. J Neurotrauma. 2002; 19(8):909-916.
[74] Leipzig N D, Xu C, Zahir T, Shoichet M S. Functional immobilization of interferon-gamma induces neuronal differentiation of neural stem cells. J Biomed Mater Res A. 2010; 93(2):625-633.
[75] Shen Y H, Shoichet M S, Radisic M. Vascular endothelial growth factor immobilized in collagen scaffold promotes penetration and proliferation of endothelial cells. Acta Biomater. 2008; 4(3):477-489.
[76] Jain A, Kim Y T, McKeon R J, Bellamkonda R V. In situ gelling hydrogels for conformal repair of spinal cord defects, and local delivery of BDNF after spinal cord injury. Biomaterials. 2006; 27(3):497-504.
[77] Talac R, Friedman J A, Moore M J, et al. Animal models of spinal cord injury for evaluation of tissue engineering treatment strategies. Biomaterials. 2004; 25(9):1505-1510.
[78] Olson H E, Rooney G E, Gross L, et al. Neural stem cell- and Schwann cell-loaded biodegradable polymer scaffolds support axonal regeneration in the transected spinal cord. Tissue Eng Part A. 2009; 15(7):1797-1805.
[79] Dadsetan M, Hefferan T E, Szatkowski J P, et al. Effect of hydrogel porosity on marrow stromal cell phenotypic expression. Biomaterials. 2008; 29(14):2193-2202.
[80] Vleggeert-Lankamp C L, de Ruiter G C, Wolfs J F, et al. Pores in synthetic nerve conduits are beneficial to regeneration. J Biomed Mater Res A. 2007; 80(4):965-982.
[81] Tsai E C, Dalton P D, Shoichet M S, Tator C H. Matrix inclusion within synthetic hydrogel guidance channels improves specific supraspinal and local axonal regeneration after complete spinal cord transection. Biomaterials. 2006; 27(3):519-533.
[82] Gros T, Sakamoto J S, Blesch A, Havton L A, Tuszynski M H. Regeneration of long-tract axons through sites of spinal cord injury using templated agarose scaffolds. Biomaterials. 2010; 31(26):6719-6729.
[83] Stokols S, Tuszynski M H. Freeze-dried agarose scaffolds with uniaxial channels stimulate and guide linear axonal growth following spinal cord injury. Biomaterials. 2006; 27(3):443-451.
[84] Gupta D, Tator C H, Shoichet M S. Fast-gelling injectable blend of hyaluronan and methylcellulose for intrathecal, localized delivery to the injured spinal cord. Biomaterials. 2006; 27(11):2370-2379.
[85] Ansorena E, De Berdt P, Ucakar B, et al. Injectable alginate hydrogel loaded with GDNF promotes functional recovery in a hemisection model of spinal cord injury. Int J Pharm. 2013; 455(1-2):148-158.
[86] Mothe A J, Tam R Y, Zahir T, Tator C H, Shoichet M S. Repair of the injured spinal cord by transplantation of neural stem cells in a hyaluronan-based hydrogel. Biomaterials. 2013; 34(15):3775-3783.
[87] Yao L, Daly W, Newland B, et al. Improved axonal regeneration of transected spinal cord mediated by multichannel collagen conduits functionalized with neurotrophin-3 gene. Gene Ther. 2013; 20(12):1149-1157.
[88] Kushchayev S V, Giers M B, Hom Eng D, et al. Hyaluronic acid scaffold has a neuroprotective effect in hemisection spinal cord injury. J Neurosurg Spine. 2016; 25(1):114-124.
[89] Amr S M, Gouda A, Koptan W T, et al. Bridging defects in chronic spinal cord injury using peripheral nerve grafts combined with a chitosan-laminin scaffold and enhancing regeneration through them by co-transplantation with bone-marrow-derived mesenchymal stem cells: case series of 14 patients. J Spinal Cord Med. 2014; 37(1):54-71.
[90] King V R, Alovskaya A, Wei D Y, Brown R A, Priestley J V. The use of injectable forms of fibrin and fibronectin to support axonal ingrowth after spinal cord injury. Biomaterials. 2010; 31(15):4447-4456.
[91] de Ruiter G C, Onyeneho I A, Liang E T, et al. Methods for in vitro characterization of multichannel nerve tubes. J Biomed Mater Res A. 2008; 84(3):643-651.
[92] Dadsetan M, Szatkowski J P, Yaszemski M J, Lu L. Characterization of photo-cross-linked oligo[poly(ethylene glycol) fumarate] hydrogels for cartilage tissue engineering. Biomacromolecules. 2007; 8(5):1702-1709.
[93] Dadsetan M, Knight A M, Lu L, Windebank A J, Yaszemski M J. Stimulation of neurite outgrowth using positively charged hydrogels. Biomaterials. 2009; 30(23-24):3874-3881.
[94] Hakim J S, Esmaeili Rad M, Grahn P J, et al. Positively Charged Oligo[Poly(Ethylene Glycol) Fumarate] Scaffold Implantation Results in a Permissive Lesion Environment after Spinal Cord Injury in Rat. Tissue Eng Part A. 2015; 21(13-14):2099-2114.
[95] Wong D Y, Leveque J C, Brumblay H, Krebsbach P H, Hollister S J, Lamarca F. Macro-architectures in spinal cord scaffold implants influence regeneration. J Neurotrauma. 2008; 25(8):1027-1037.
[96] Yao L, Wang S, Cui W, et al. Effect of functionalized micropatterned PLGA on guided neurite growth. Acta Biomater. 2009; 5(2):580-588.
[97] Goldner J S, Bruder J M, Li G, Gazzola D, Hoffman-Kim D. Neurite bridging across micropatterned grooves. Biomaterials. 2006; 27(3):460-472.
[98] Miller C, Jeftinija S, Mallapragada S. Synergistic effects of physical and chemical guidance cues on neurite alignment and outgrowth on biodegradable polymer substrates. Tissue Eng. 2002; 8(3):367-378.
[99] Turney S G, Bridgman P C. Laminin stimulates and guides axonal outgrowth via growth cone myosin II activity. Nat Neurosci. 2005; 8(6):717-719.
[100] Adams D N, Kao E Y, Hypolite C L, Distefano M D, Hu W S, Letourneau P C. Growth cones turn and migrate up an immobilized gradient of the laminin IKVAV peptide. J Neurobiol. 2005; 62(1):134-147.
[101] Dodla M C, Bellamkonda R V. Anisotropic scaffolds facilitate enhanced neurite extension in vitro. J Biomed Mater Res A. 2006; 78(2):213-221.
[102] Fan Z, Shen Y, Zhang F, et al. Control of olfactory ensheathing cell behaviors by electrospun silk fibroin fibers. Cell Transplant. 2013; 22 Suppl 1:S39-50.
[103] Liu T, Houle J D, Xu J, Chan B P, Chew S Y. Nanofibrous collagen nerve conduits for spinal cord repair. Tissue Eng Part A. 2012; 18(9-10):1057-1066.
[104] Hodde D, Gerardo-Nava J, Wohlk V, et al. Characterisation of cell-substrate interactions between Schwann cells and three-dimensional fibrin hydrogels containing orientated nanofibre topographical cues. Eur J Neurosci. 2016; 43(3):376-387.
[105] Tsintou M, Dalamagkas K, Seifalian A M. Advances in regenerative therapies for spinal cord injury: a biomaterials approach. Neural Regen Res. 2015; 10(5):726-742.
[106] Singh S, Bandini S B, Donnelly P E, Schwartz J, Schwarzbauer J E. A cell-assembled, spatially aligned extracellular matrix to promote directed tissue development. J Mater Chem B. 2014; 2(11):1449-1453.
[107] Frantz C, Stewart K M, Weaver V M. The extracellular matrix at a glance. J Cell Sci. 2010; 123(Pt 24):4195-4200.
[108] Haggerty A E, Marlow M M, Oudega M. Extracellular matrix components as therapeutics for spinal cord injury. Neurosci Lett. 2017; 652:50-55.
[109] Suzuki H, Kanchiku T, Imajo Y, et al. Artificial collagen-filament scaffold promotes axon regeneration and long tract reconstruction in a rat model of spinal cord transection. Med Mol Morphol. 2015; 48(4):214-224.
[110] Tom V J, Doller C M, Malouf A T, Silver J. Astrocyte-associated fibronectin is critical for axonal regeneration in adult white matter. J Neurosci. 2004; 24(42):9282-9290.
[111] Ma Z, Mao Z, Gao C. Surface modification and property analysis of biomedical polymers used for tissue engineering. Colloids Surf B Biointerfaces. 2007; 60(2):137-157.
[112] Cukierman E, Pankov R, Stevens D R, Yamada K M. Taking cell-matrix adhesions to the third dimension. Science. 2001; 294(5547):1708-1712.
[113] Mao Y, Schwarzbauer J E. Stimulatory effects of a three-dimensional microenvironment on cell-mediated fibronectin fibrillogenesis. J Cell Sci. 2005; 118(Pt 19):4427-4436.
[114] Volpato F Z, Fuhrmann T, Migliaresi C, Hutmacher D W, Dalton P D. Using extracellular matrix for regenerative medicine in the spinal cord. Biomaterials. 2013; 34(21):4945-4955.
[115] Sharp J, Frame J, Siegenthaler M, Nistor G, Keirstead H S. Human embryonic stem cell-derived oligodendrocyte progenitor cell transplants improve recovery after cervical spinal cord injury. Stem Cells. 2010; 28(1):152-163.
[116] Richardson P M, McGuinness U M, Aguayo A J. Axons from CNS neurons regenerate into PNS grafts. Nature. 1980; 284(5753):264-265.
[117] Li Y, Raisman G. Schwann cells induce sprouting in motor and sensory axons in the adult rat spinal cord. J Neurosci. 1994; 14(7):4050-4063.
[118] Biernaskie J, Sparling J S, Liu J, et al. Skin-derived precursors generate myelinating Schwann cells that promote remyelination and functional recovery after contusion spinal cord injury. J Neurosci. 2007; 27(36):9545-9559.
[119] Park H W, Lim M J, Jung H, Lee S P, Paik K S, Chang M S. Human mesenchymal stem cell-derived Schwann cell-like cells exhibit neurotrophic effects, via distinct growth factor production, in a model of spinal cord injury. Glia. 2010; 58(9):1118-1132.
[120] Daniloff J K, Levi G, Grumet M, Rieger F, Edelman G M. Altered expression of neuronal cell adhesion molecules induced by nerve injury and repair. J Cell Biol. 1986; 103(3):929-945.
[121] Cornbrooks C J, Carey D J, McDonald J A, Timpl R, Bunge R P. In vivo and in vitro observations on laminin production by Schwann cells. Proc Natl Acad Sci USA. 1983; 80(12):3850-3854.
[122] Carey D J, Eldridge C F, Cornbrooks C J, Timpl R, Bunge R P. Biosynthesis of type IV collagen by cultured rat Schwann cells. J Cell Biol. 1983; 97(2):473-479.
[123] Kanno H, Pressman Y, Moody A, et al. Combination of engineered Schwann cell grafts to secrete neurotrophin and chondroitinase promotes axonal regeneration and locomotion after spinal cord injury. J Neurosci. 2014; 34(5):1838-1855.
[124] Anderson K D, Guest J D, Dietrich W D, et al. Safety of Autologous Human Schwann Cell Transplantation in Subacute Thoracic Spinal Cord Injury. J Neurotrauma. 2017; 34(21):2950-2963.
[125] Saberi H, Moshayedi P, Aghayan H R, et al. Treatment of chronic thoracic spinal cord injury patients with autologous Schwann cell transplantation: an interim report on safety considerations and possible outcomes. Neurosci Lett. 2008; 443(1):46-50.
[126] Reyes M, Lund T, Lenvik T, Aguiar D, Koodie L, Verfaillie C M. Purification and ex vivo expansion of postnatal human marrow mesodermal progenitor cells. Blood. 2001; 98(9):2615-2625.
[127] Wright K T, El Masri W, Osman A, Chowdhury J, Johnson W E. Concise review: Bone marrow for the treatment of spinal cord injury: mechanisms and clinical applications. Stem Cells. 2011; 29(2):169-178.
[128] Menezes K, Nascimento M A, Goncalves J P, et al. Human mesenchymal cells from adipose tissue deposit laminin and promote regeneration of injured spinal cord in rats. PLoS One. 2014; 9(5):e96020.
[129] Caplan A I. Why are MSCs therapeutic? New data: new insight. J Pathol. 2009; 217(2):318-324.
[130] Crigler L, Robey R C, Asawachaicharn A, Gaupp D, Phinney D G. Human mesenchymal stem cell subpopulations express a variety of neuro-regulatory molecules and promote neuronal cell survival and neuritogenesis. Exp Neurol. 2006; 198(1):54-64.
[131] Bartholomew A, Sturgeon C, Siatskas M, et al. Mesenchymal stem cells suppress lymphocyte proliferation in vitro and prolong skin graft survival in vivo. Exp Hematol. 2002; 30(1):42-48.
[132] Krampera M, Glennie S, Dyson J, et al. Bone marrow mesenchymal stem cells inhibit the response of naive and memory antigen-specific T cells to their cognate peptide. Blood. 2003; 101(9):3722-3729.
[133] Beyth S, Borovsky Z, Mevorach D, et al. Human mesenchymal stem cells alter antigen-presenting cell maturation and induce T-cell unresponsiveness. Blood. 2005; 105(5):2214-2219.
[134] Badner A, Vawda R, Laliberte A, et al. Early Intravenous Delivery of Human Brain Stromal Cells Modulates Systemic Inflammation and Leads to Vasoprotection in Traumatic Spinal Cord Injury. Stem Cells Transl Med. 2016; 5(8):991-1003.
[135] Chen J, Li Y, Katakowski M, et al. Intravenous bone marrow stromal cell therapy reduces apoptosis and promotes endogenous cell proliferation after stroke in female rat. J Neurosci Res. 2003; 73(6):778-786.
[136] Geffner L F, Santacruz P, Izurieta M, et al. Administration of autologous bone marrow stem cells into spinal cord injury patients via multiple routes is safe and improves their quality of life: comprehensive case studies. Cell Transplant. 2008; 17(12):1277-1293.
[137] Oraee-Yazdani S, Hafizi M, Atashi A, et al. Co-transplantation of autologous bone marrow mesenchymal stem cells and Schwann cells through cerebral spinal fluid for the treatment of patients with chronic spinal cord injury: safety and possible outcome. Spinal Cord. 2016; 54(2):102-109.
[138] Staff N P, Madigan N N, Morris J, et al. Safety of intrathecal autologous adipose-derived mesenchymal stromal cells in patients with ALS. Neurology. 2016; 87(21):2230-2234.
[139] Dennes T J, Hunt G C, Schwarzbauer J E, Schwartz J. High-yield activation of scaffold polymer surfaces to attach cell adhesion molecules. J Am Chem Soc. 2007; 129(1):93-97.
[140] Dennes T J, Schwartz J. A nanoscale adhesion layer to promote cell attachment on PEEK. J Am Chem Soc. 2009; 131(10):3456-3457.
[141] Dai L G, Huang G S, Hsu S H. Sciatic nerve regeneration by cocultured Schwann cells and stem cells on microporous nerve conduits. Cell Transplant. 2013; 22(11):2029-2039.
[142] Zhou L N, Cui X J, Su K X, Wang X H, Guo J H. Beneficial reciprocal effects of bone marrow stromal cells and Schwann cells from adult rats in a dynamic coculture system in vitro without intercellular contact. Mol Med Rep. 2015; 12(4):4931-4938.
[143] Yang E Z, Zhang G W, Xu J G, et al. Multichannel polymer scaffold seeded with activated Schwann cells and bone mesenchymal stem cells improves axonal regeneration and functional recovery after rat spinal cord injury. Acta Pharmacol Sin. 2017; 38(5):623-637.
[144] Ban D X, Ning G Z, Feng S Q, et al. Combination of activated Schwann cells with bone mesenchymal stem cells: the best cell strategy for repair after spinal cord injury in rats. Regen Med. 2011; 6(6):707-720.
[145] Levorson E J, Santoro M, Kasper F K, Mikos A G. Direct and indirect co-culture of chondrocytes and mesenchymal stem cells for the generation of polymer/extracellular matrix hybrid constructs. Acta Biomater. 2014; 10(5):1824-1835.
[146] Bian L, Zhai D Y, Mauck R L, Burdick J A. Coculture of human mesenchymal stem cells and articular chondrocytes reduces hypertrophy and enhances functional properties of engineered cartilage. Tissue Eng Part A. 2011; 17(7-8):1137-1145.
[147] Poitelon Y, Bogni S, Matafora V, et al. Spatial mapping of juxtacrine axo-glial interactions identifies novel molecules in peripheral myelination. Nat Commun. 2015; 6:8303.
[148] Belin S, Zuloaga K L, Poitelon Y. Influence of Mechanical Stimuli on Schwann Cell Biology. Front Cell Neurosci. 2017; 11:347.
[149] Omar M H, Kerrisk Campbell M, Xiao X, et al. CNS Neurons Deposit Laminin alpha5 to Stabilize Synapses. Cell Rep. 2017; 21(5):1281-1292.

The citation of any document or reference is not to be construed as an admission that it is prior art with respect to the present invention.

### Example 2

Hydrogels are ubiquitous materials of use ranging from fundamental studies of cell behavior¹⁻⁷ to current and future applications in biomedical devices;^{8,9} especially exciting is their potential for implementation as scaffolds to address neural injuries.^{4,5,7,10-15} In many cases, the hydrogel is based on a synthetic (e.g., polyethylene glycol or polyacrylate) or a natural (e.g., alginate) hydrophilic polymer;^{16,17} utilization of such hydrogels might require adjusting their surface properties to affect, for example, their hydrophilicity, or to control cell adhesion and spreading. Many elegant methods have been developed to these ends, in which surface modification of a cell-nonadhesive hydrogel may involve, for example, introduction of exogenous, cell-adhesive functional groups,^{6,18} large molecule surface adsorption on preformed hydrogels,^{3,4,7,8,12,13,19-22} or incorporation of cell-adhesive monomers;^{5,6,9,21,23-25} some require specialized equipment or conditions.^{2,23,25-28} In contrast, the method we describe herein requires no modification of the polymer components of the hydrogel relies only on its water content.

We have reported that vapor deposition of Ti and Zr

can be used to prepare thin oxide films on polymers containing organic functionality that can coordinate with these metal ions. These thin films can then be used to control the surface chemistry of the polymer by serving as reactive interfaces to bond phosphonate monolayers. This method, which has been used on polyamides,²⁹ polyimides,³⁰ polyketones,³¹ polyurethanes,³² and polyesters,^{31,33} fails in the derivatization of a hydrated hydrogel: rapid dehydration occurs under the reduced pressure conditions of vapor deposition, and the water thus released destroys the alkoxide reagent in the vapor phase. Here, we show that a thin Ti or Zr oxide coating can be rapidly synthesized on a fully hydrated hydrogel by exploiting its very water content in a process reminiscent of the use of surface water for atomic layer deposition of TiO₂.³⁴ Our approach requires no specialized techniques or materials,^{27,28,35} but simply involves the use of a hydrophobic solvent. We report that this oxide coating serves as a reactive interface for bonding phosphonic acids, yielding the first examples of a self-assembled phosphonate monolayer (SAMP) growth on a hydrated material. We also show that patterning the SAMP on the hydrogel can be effected through a sequence involving physical imprinting to template cell adhesion and spreading on its surface in spatially defined patterns.

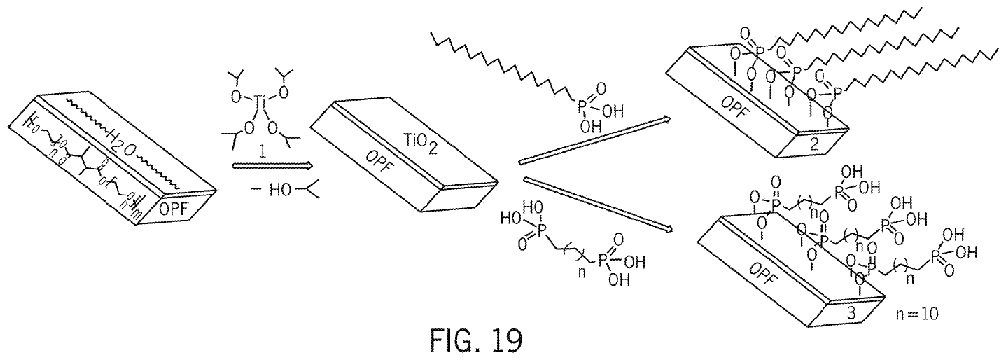

We chose to focus on the surface chemistry of oligo-(polyethylene glycol) fumarate (OPF⁵) because of its potential for use in neural regeneration devices.¹⁵ It is an especially challenging substrate in that it is devoid of functionality for covalent attachment of surface adducts; it is replete, though, with oxy and carbonyl groups capable of metal ion complex-ation.²⁹⁻³² Partially dehydrated, 0.08 mm thick, friable sheets of OPF were provided by Dr. Michael Yaszemski and Nicholas Madigan (Mayo Clinic, Rochester, Minn.); they swell on rehydration in Milli-Q water (under these conditions, a 4 mm×4 mm coupon of “dry” OPF swells to about 12 mm×12 mm). In a typical experiment, a fully rehydrated coupon is razor cut to 1 cm×1 cm, blotted dry of excess water, and then put in a solution of 30 μL titanium iso-propoxide (1) in 5 mL of toluene (hexane or xylene are comparable for use) for about 1 min. A rapid reaction occurs at the coupon surface, presumably by hydrolysis with hydrogel-supplied water, and cross-linking³⁶ of initially formed hydroxytitanium species gives a thin surface coating of white titanium dioxide,³⁴ identified by EDS analysis. (If a hydrophilic solvent such as iso-propanol is used instead, rapid hydrolysis occurs in the supernatant as well as at the coupon surface and the coupon physically distorts, both apparently by solvent-assisted partial dehydration of the hydrogel; the precipitated TiO₂ coating³⁶ then easily spalls from the surface.) The coated coupon is then removed from the solution, gently rinsed with iso-propanol, and heated on a glass slide to 30-35° C. for about 1 min to further cross-link surface Ti species. No ostensible physical changes are observed with heating at this temperature, but samples curl noticeably if heated >40° C. Zirconium iso-propoxide (1 mg in 5 mL of toluene) was used to make the analogous ZrO₂ thin layer. TiO₂- or ZrO₂-terminated coupons are then immersed in a solution of a phosphonic acid for several hours, rinsed, and warmed to 35° C. for 1 min to ensure the formation of the phosphonate monolayer (2 or 3; FIG. 19).

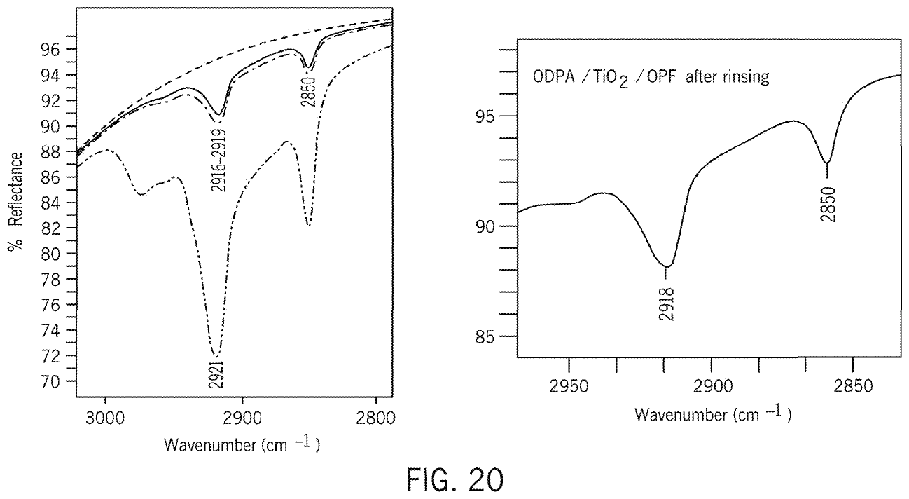

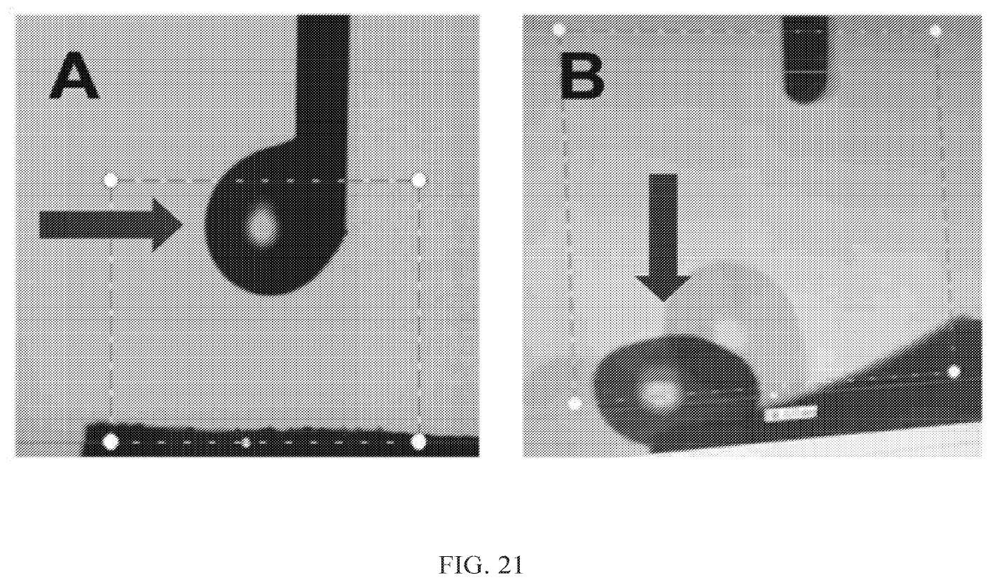

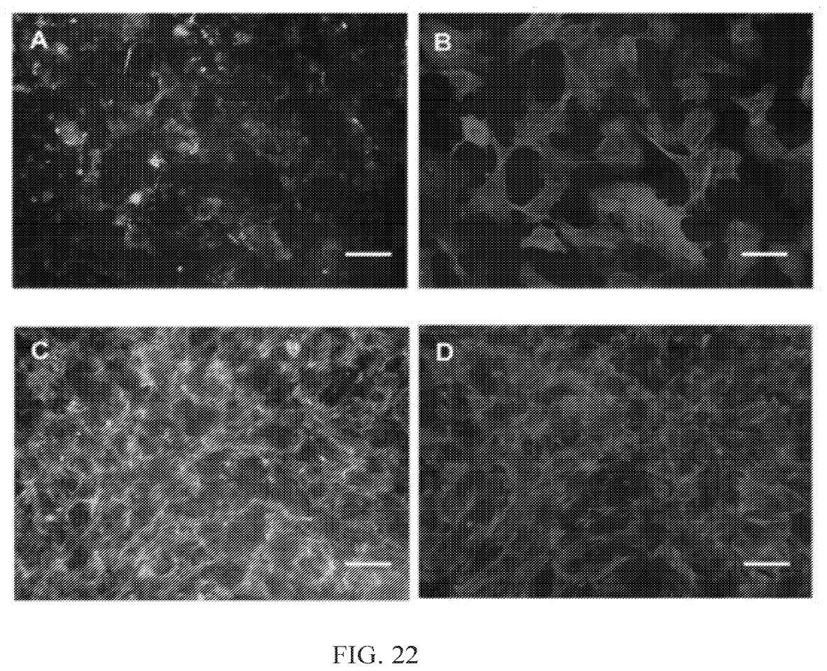

Monolayers of octadecylphosphonate (ODPA) do not adhere to OPF in the absence of the Ti(Zr)O₂ interface. ODPA prepared on TiO₂/OPF (using 2.5 mg of octadecyl-phosphonic acid/5 mL toluene or xylene) served as a diagnostic of constituent molecular order and stability under aqueous conditions; water wetting contact angle measurements and infrared (IR) spectroscopy were both assessed by comparison with ODPA monolayers previously prepared on Ti native oxide TiO₂.³⁷ In particular, IR of ODPA/TiO₂ showed ν_{CH antisymm}=2914 cm⁻¹, indicating a highly ordered film of all-trans alkylphosphonates with few gauche defects;^{38,39} areal molecular loading of ODPA was measured by quartz crystal microgravimetry (QCM) to be 1.18 nm/cm² (corresponding to ca. 14 Å²/molecule), and corroborating that the monolayers consisted of closely packed alkylphosphonate chains.⁴⁰ ODPA was also grown on 1-8 nm thick samples of ZrO₂ that were synthesized from vapor-deposited Zr tetra(tert-butoxide) on SiO₂;⁴¹ it was measured by QCM to have a real molecular loadings of 0.96±0.02 nmol/cm², or about 17 Å²/molecule. Given that IR spectra recorded on six coupons of 2 showed ν_{CH antisymm}=2918-2915 cm⁻¹ (FIG. 20), we that well-ordered, closely packed ODPA is formed TiO₂/OPF. Similar IR observations were made using partially dehydrated OPF that was treated with 1 or its Zr analog in toluene. To determine the stability of the oxide interface-phosphonate ensemble under conditions necessary for cell growth, IR was used to examine the effects of ethanol-rinsing on treated coupons and on their immersion in phosphate-buffered saline (PBS) for up to 7 days. Neither procedure resulted in a significant loss in IR signal intensity or change in position for this characteristic indicator of film content or order after the weakly adsorbed multilayer was rinsed away.

The hydrophobicity imparted to the ODPA/TiO₂-terminated, fully hydrated hydrogel was shown in two ways: the coupon floated on the surface of the PBS, and it resisted contact angle measurements for water wetting because drops of water would not transfer easily from the blunt needle tip of the water-filled syringe to the ODPA-terminated surface. Indeed, if the tip were placed close to the surface, the test drop of water would literally bounce off the surface back onto the tip (a greater tip—surface separation enabled this measurement to be >115°); a water droplet simply rolled off a slightly tilted coupon surface (FIG. 21). Although hydrophobic hydrogels have been reported by coating the surface with a layer of hydrophobic microparticles⁴² or by alkylation of exposed, pendant amino groups,⁴³ we believe that our method is the first to show that superhydrophobicity imparted by a self-assembled monolayer formation on a simple, pegylated hydrogel.

OPF is not cell-adhesive: in a control test, we find that few fibroblasts attached to it and did not spread over 24 h. It was therefore of interest to determine if our SAMP/Ti(Zr)O₂/hydrogel method could quickly render OPF cell-adhesive. Adapting our procedure for such surface activation of hydrophobic polymers via a TiO₂ adhesion layer,^{44,45} we immersed coupons of TiO₂/OPF in solutions of (1,12-diphos-phono)dodecane (2.5 mg diphosphonoalkane/5 mL toluene) under the same conditions used to prepare monolayers of ODPA. Phosphonic acid-terminated monolayers (3)^{31,33,44,46} thus obtained have been shown to be cell-adhesive in vitro using our oxide thin films to bond them onto several polymers^{31,33,44} and in vivo on Ti oxide-terminated Ti metal.⁴⁷ NIH 3T3 fibroblasts were plated on the SAMP of (1,12-diphosphono)dodecane, and were analyzed after 1, 2, and 4 days in culture (FIG. 22). Cells attached, spread, and formed actin stress fibers within 1 day of plating. They proliferated to confluence and assembled a fibrillar fibronectin matrix after 2 days, which became more robust by day 4 in culture (FIG. 22). We decellularized cultures^{33,45} grown on 3, yielding a cell-assembled extracellular matrix on the OPF hydrogel. The integrity of the matrix was monitored by immunostaining fibronectin fibrils and showed that their organization was stable in PBS at 4° C. for at least 26 days (Figure S4B). The lack of 4′,6-diamidino-2-phenylindole (DAPI) staining in these samples indicates the absence of nuclear material.

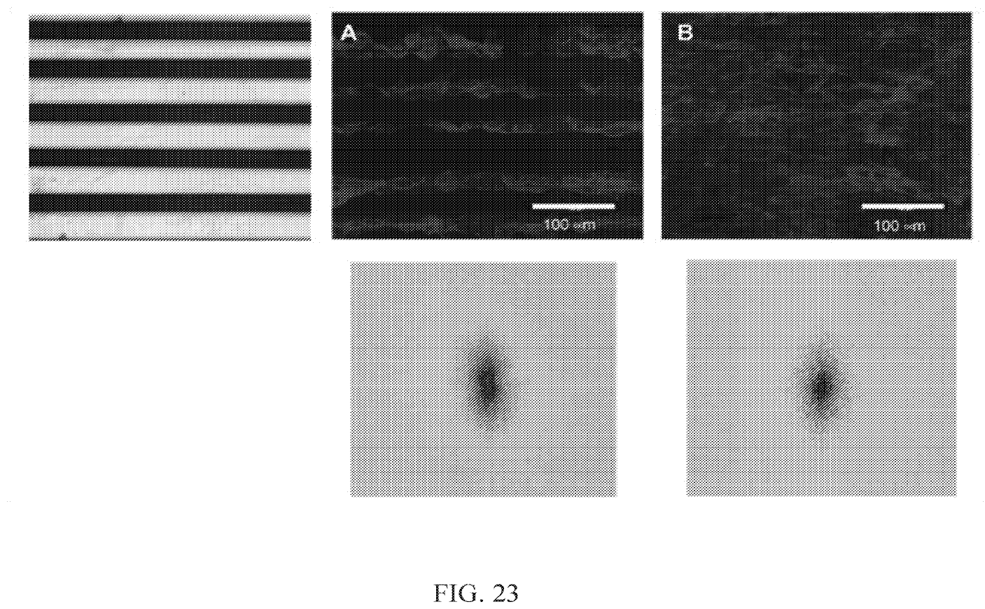

We recently showed⁴⁵ that the neurite outgrowth from dorsal root ganglia plated on cell-assembled, decellularized extracellular matrix (ECM) was in alignment with photolitho-graphically patterned SAMP/TiO₂/poly-ethylene terephthalate OPF is, however, incompatible with photolithography; thus, we created a new method for its surface patterning. First, masks were prepared from 25 μm thick Kapton sheets by laser-cutting 30 μm wide stripes that were separated by unablated stripes, also 30 μm wide (30×30). In a typical patterning procedure, OPF was then cut into 5 mm×5 mm squares that were hydrated to form 1 cm×1 cm coupons, which were then dried on filter paper. A small drop of water was placed on a glass slide, and a coupon was placed on the water to hold it in place. Any excess water was blotted from the OPF surface. The Kapton mask was then placed onto the coupon such that the striped areas were directly in contact with the OPF surface. The OPF-mask composite was heated to 30° C. on a hot plate, and the mask was then depressed into the OPF (debossing) using a 200 g weight; heating with the mask in place was continued for 15 s. Any visible water was blotted from the surface of the OPF-mask ensemble, which was then placed into the vial containing either the Ti or Zr alkoxide precursor solution prepared as described above, and was left immersed for 30 s. The ensemble was removed from this solution, rinsed three times with toluene, and was then heated to 30° C. for 30 s; this creates a layer of cross-linked oxide that is patterned on the OPF surface: only the regions of the OPF that are not covered by the Kapton shadow mask (the ablated stripes) are exposed to the solution of the Ti or Zr alkoxide. The ensemble was removed from heat and allowed to cool for 15 s before being immersed in the phosphonic acid solution described above, which yields the cell-adhesive stripes on the oxide-covered areas of the OPF surface. The ensemble remained in this solution for about 12 h, and was then rinsed successively with toluene, isopropanol, and water. The Kapton mask and glass slide support were separated mechanically from the OPF; often, they detach spontaneously during the rinsing process. The coupon of now-patterned OPF (FIG. 23) was heated on a temperature-controlled hot plate at 30° C. for 30 s. Freshly prepared, patterned OPF coupons were stored either in water or PBS prior to plating with NIH 3T3 cells, as was done for unpatterned material. Fast Fourier transform (FFT) analyses^{4,19} of the cell and matrix alignment on patterned OPF were done with ImageJ or Fiji software; the orientation direction of the FFT obtained in this way is 90° rotated from the direction of the pattern,⁵⁰ and outputs were used to determine the fidelity of cell alignment and spreading with the debossed pattern (FIG. 23). In a control experiment, we showed that debossed OPF that is completely covered with 3 yields unaligned cell attachment and spreading.

We have shown that a two-step synthesis involving a water-sensitive reagent can be used to prepare a thin oxide layer on a fully hydrated hydrogel: we utilize the water of the hydrogel, itself, as a reagent to hydrolyze a reactive Ti or Zr alkoxide that is delivered to the hydrogel surface in a hydrophobic solvent. Deposition and cross-linking of the thin coating of TiO₂ or ZrO₂ occurs on the hydrogel surface; no covalent bonding to the hydrogel surface is required. We also showed that monolayers of organophosphonates can be grown on these oxides that serve as reactive interfaces. Through choice of phosphonic acid, a hydrophobic or, especially, a cell-adhesive phosphonate termination can be imparted to the otherwise hydrophilic, cell-nonadhesive hydrogel; the hydrophobic SAMP/Ti(Zr)O₂/OPF ensemble provided us the means to study interface stability by IR. We also showed that this adhesion layer can be patterned on the OPF surface using a shadow mask that is “debossed” into its surface, leaving only laser-cut slits that are exposed to the metal alkoxide reagent; this phosphonate pattern then templates co-aligned cell spreading. Given the simplicity of our methods and the versatility that is derived from the structural variety of available hydrogels and phosphonic acids, we believe that our oxide/SAMP approach to the control of hydrogel surface properties beneficially augments the repertory of existing routes reported for surface treatment of hydrogels. In that regard, studies using spatially patterned SAMP/TiO₂/OPF are underway to determine if an aligned, cell-assembled matrix is beneficial for bioactive neural repair devices in vivo.

### REFERENCES FOR EXAMPLE 2

(1) Caliari, S. R.; Burdick, J. A. A practical guide to hydrogels for cell culture. Nat. Methods 2016, 13, 405-414.
(2) Patel, N. R.; Whitehead, A. K.; Newman, J. J.; Caldorera-Moore, M. E. Poly(ethylene glycol) Hydrogels with Tailorable Surface and Mechanical Properties for Tissue Engineering Applications. ACS Biomater. Sci. Eng. 2017, 3, 1494-1498.
(3) Yan, T.; Sun, R.; Li, C.; Tan, B.; Mao, X.; Ao, N. Immobilization of type-I collagen and basic fibroblast growth factor (bFGF) onto poly (HEMA-co-MMA) hydrogel surface and its cytotoxicity study. J. Mater. Sci.: Mater. Med. 2010, 21, 2425-2433.
(4) Morin, E. A.; Tang, S.; Rogers, K. L.; He, W. Facile Use of Cationic Hydrogel Particles for Surface Modification of Planar Substrates Toward Multifunctional Neural Permissive Surfaces: An in Vitro Investigation. ACS Biomater. Sci. Eng. 2016, 8, 5737-5745.
(5) Dadsetan, M.; Knight, A. M.; Lu, L.; Windebank, A. J.; Yaszemski, M. J. Stimulation of neurite outgrowth using positively charged hydrogels. Biomaterials 2009, 30, 3874-3881.
(6) Leslie-Barbick, J. E.; Moon, J. J.; West, J. L. Covalently-Immobilized Vascular Endothelial Growth Factor Promotes Endo-thelial Cell Tubulogenesis in Poly(ethylene glycol) Diacrylate Hydrogels. J. Biomater. Sci., Polym. Ed. 2009, 20, 1763-1779.
(7) Hejěl, A.; Růžička, J.; Kapcalova\ddot{,}M.; Turnovcova\ddot{,}K.; Krumbholcova\ddot{,}E.; Pr\check{a}dny\acute{,}M.; Michaĺek, J.; Cihlaŕ, J.; Jendelova\acute{,} P.; Sykova\acute{,}E. Adjusting the Chemical and Physical Properties of Hydrogels Leads to Improved Stem Cell Survival and Tissue Ingrowth in Spinal Cord Injury Reconstruction: A Comparative Study of Four Methacrylate Hydrogels. Stem Cells Dev. 2013, 22, 2794-2805.
(8) Xiang, J.; Sun, J.; Hong, J.; Wang, W.; Wei, A.; Le, Q.; Xu, J. T-style keratoprosthesis based on surface-modified poly (2-hydroxyethyl methacrylate) hydrogel for cornea repairs. Mater. Sci. Eng., C 2015, 50, 274-285.
(9) Dadsetan, M.; Giuliani, M.; Wanivenhaus, F.; Brett Runge, M.; Charlesworth, J. E.; Yaszemski, M. J. Incorporation of phosphate group modulates bone cell attachment and differentiation on oligo(polyethylene glycol) fumarate hydrogel. Acta Biomater. 2012, 8, 1430-1439.
(10) Tsintou, M.; Dalamagkas, K.; Seifalian, A. M. Advances in regenerative therapies for spinal cord injury: a biomaterials approach. Neural Regener. Res. 2015, 10, 726-742.
(11) Assunca̧õ-Silva, R. C.; Gomes, E. E.; Sousa, N.; Silva, N. A.; Salgado, A. J. Hydrogels and Cell Based Therapies in Spinal Cord Injury Regeneration. Stem Cells Int. 2015, 2015, 948040.
(12) Haile, Y.; Berski, S.; Dra\ddot{g}er, G.; Nobre, A.; Stummeyer, K.; Gerardy-Schahn, R.; Grothe, C. The effect of modified polysialic acid based hydrogels on the adhesion and viability of primary neurons and glial cells. Biomaterials 2008, 29, 1880-1891.
(13) Shanbhag, M. S.; Lathia, J. D.; Mughal, M. R.; Francis, N. L. Neural Progenitor Cells Grown on Hydrogel Surfaces Respond to the Product of the Transgene of Encapsulated Genetically Engineered Fibroblasts. Biomacromolecules 2010, 11, 2936-2943.
(14) Chen, B. K.; Madigan, N. N.; Hakim, J. S.; Dadsetan, M.; McMahon, S. S.; Yaszemski, M. J.; Windebank, A. J. GDNF Schwann cells in hydrogel scaffolds promote regional axon regeneration, remyelination and functional improvement after spinal cord trans-ection in rats. J. Tissue Eng. Regener. Med. 2018, 12, e398-e407.
(15) Hakim, J. S.; Esmaeili Rad, M.; Grahn, P. J.; Chen, B. K.; Knight, A. M.; Schmeichel, A. M.; Isaq, N. A.; Dadsetan, M.; Yaszemski, M. J.; Windebank, A. J. Positively Charged Oligo[Poly- (Ethylene Glycol) Fumarate] Scaffold Implantation Results in a Permissive Lesion Environment after Spinal Cord Injury in Rat. Tissue Eng., Part A 2015, 21, 2099-2114.
(16) Drury, J. L.; Mooney, D. J. Hydrogels for tissue engineering: scaffold design variables and applications. Biomaterials 2003, 24, 4337-4351.
(17) Van Vlierberghe, S.; Dubruel, P.; Schacht, E. Biopolymer-Based Hydrogels As Scaffolds for Tissue Engineering Applications: A Review. Biomacromolecules 2011, 12, 1387-1408.
(18) Zainuddin; Barnard, Z.; Keen, I.; Hill, D. J. T.; Chirila, T. V.; Harkin, D. G. PHEMA Hydrogels Modified through the Grafting of Phosphate Groups by ATRP Support the Attachment and Growth of Human Corneal Epithelial Cells. J. Biomater. Appl. 2008, 23, 147-168.
(19) Brynda, E.; Houska, M.; Kysilka, J.; Pr\check{a}\acute{d}ny\acute{,}M.; Lesny\acute{,}P.; Jendelova\acute{,}P.; Michaĺek, J.; Sykova\acute{,}E. Surface modification of hydrogels based on poly(2-hydroxyethyl methacrylate) with extracellular matrix proteins. J. Mater. Sci.: Mater. Med. 2009, 20, 909-915.
(20) Kim, M.; Kim, Y.-J.; Gwon, K.; Tae, G. Modulation of Cell Adhesion of Heparin-Based Hydrogel by Efficient Physisorption of Adhesive Proteins. Macromol. Res. 2012, 20, 271-276.
(21) Best, J. P.; Javed, S.; Richardson, J. J.; Cho, K. L.; Kamphuis, M. M. J.; Caruso, F. Stiffness-mediated adhesion of cervical cancer cells to soft hydrogel films. Soft Matter 2013, 9, 4580-4584.
(22) Al-Haque, S.; Miklas, J. W.; Feric, N.; Chiu, L. L. Y.; Chen, W. L. K.; Simmons, C. A.; Radisic, M. Hydrogel Substrate Stiffness and Topography Interact to Induce Contact Guidance in Cardiac Fibroblasts. Macromol. Biosci. 2012, 12, 1342-1353.
(23) Camci-Unal, G.; Aubin, H.; Ahari, A. F.; Bae, H.; Nichol, J. W.; Khademhosseini, A. Surface-modified hyaluronic acid hydrogels to capture endothelial progenitor cells. Soft Matter 2010, 6, 5120-5126.
(24) Ravi, S.; Krishnamurthy, V. R.; Caves, J. M.; Haller, C. A.; Chaikof, E. L. Maleimide-thiol coupling of a bioactive peptide to an elastin-like protein polymer. Acta Biomater. 2012, 8, 627-635.
(25) Chen, R. T.; Marchesan, S.; Evans, R. A.; Styan, K. E.; Such, G. K.; Postma, A.; McLean, K. M.; Muir, B. W.; Caruso, F. Photoinitiated Alkyne-Azide Click and Radical Cross-Linking Reactions for the Patterning of PEG Hydrogels. Biomacromolecules 2012, 13, 889-895.
(26) Kim, D.-N.; Park, J.; Koh, W.-G. Control of cell adhesion on poly(ethylene glycol) hydrogel surfaces using photochemical modification and micropatterning techniques. J. Ind. Eng. Chem. 2009, 15, 124-128.
(27) Zhang, Z.; Lai, Y.; Yu, L.; Ding, J. Effects of immobilizing sites of RGD peptides in amphiphilic block copolymers on efficacy of cell adhesion. Biomaterials 2010, 31, 7873-7882.
(28) Kolaŕ\check{o}va\acute{,}K.; Kasaĺkova\acute{,}N.; Dvor\check{a}ńkova\acute{,}B.; Michaĺek, J.; Švorc\check{i}k, V. Surface modification of hydrogels and cell adhesion. Mater. Sci. Forum 2008, 567-568, 265-268.
(29) Dennes, T. J.; Hunt, G. C.; Schwarzbauer, J. E.; Schwartz, J. High-Yield Activation of Scaffold Polymer Surfaces To Attach Cell Adhesion Molecules. J. Am. Chem. Soc. 2007, 129, 93-97.
(30) Dennes, T. J.; Schwartz, J. A Nanoscale Metal Alkoxide/Oxide Adhesion Layer Enables Spatially Controlled Metallization of Polymer Surfaces. ACS Appl. Mater. Interfaces 2009, 1, 2119-2122.
(31) Dennes, T. J.; Schwartz, J. A Nanoscale Adhesion Layer to Promote Cell Attachment on PEEK. J. Am. Chem. Soc. 2009, 131, 3456-3457.
(32) Dennes, T. J.; Schwartz, J. Controlling cell adhesion on polyurethanes. Soft Matter 2008, 4, 86-89.
(33) Singh, S.; Bandini, S. B.; Donnelly, P. E.; Schwartz, J.; Schwarzbauer, J. E. A cell-assembled, spatially aligned extracellular matrix to promote directed tissue development. J. Mater. Chem. B 2014, 2, 1449-1453.
(34) Reinke, M.; Kuzminykh, Y.; Hoffmann, P. Surface Reaction Kinetics of Titanium Isopropoxide and Water in Atomic Layer Deposition. J. Phys. Chem. C 2016, 120, 4337-4344.
(35) Zan, X.; Kozlov, M.; McCarthy, T. J.; Su, Z. Covalently Attached, Silver-Doped Poly(vinyl alcohol) Hydrogel Films on Poly(L-lactic acid). Biomacromolecules 2010, 11, 1082-1088.
(36) Dunuwila, D. D.; Gagliardi, C. D.; Berglund, K. A. Application of Controlled Hydrolysis of Titanium(IV) Isopropoxide to Produce Sol—Gel-Derived Thin Films. Chem. Mater. 1994, 6, 1556-1562.
(37) Gawalt, E. S.; Avaltroni, M. J.; Koch, N.; Schwartz, J. Self-Assembly and Bonding of Alkanephosphonic Acids on the Native Oxide Surface of Titanium. Langmuir 2001, 17, 5736-5738.
(38) Schwartz, J.; Avaltroni, M. J.; Danahy, M. P.; Silverman, B. M.; Hanson, E. L.; Schwarzbauer, J. E.; Midwood, K. S.; Gawalt, E. S. Cell attachment and spreading on metal implant materials. Mater. Sci. Eng., C 2003, 23, 395-400.
(39) Gao, W.; Dickinson, L.; Grozinger, C.; Morin, F. G.; Reven, L. Self-Assembled Monolayers of Alkylphosphonic Acids on Metal Oxides. Langmuir 1996, 12, 6429-6435.
(40) Kitaigorodskii, A. I. Organic Chemical Crystallography; Consultants Bureau: New York, 1961; p 541.
(41) Dennes, T. J., III. Spatially-Controlled Surface Activation of Preformed Polymers. Ph.D. Dissertation, Princeton University, Ann Arbor, 2008.
(42) Oliveira, N. M.; Zhang, Y. S.; Ju, J.; Chen, A.-Z.; Chen, Y.; Sonkusale, S. R.; Dokmeci, M. R.; Reis, R. L.; Mano, J. F.; Khademhosseini, A. Hydrophobic Hydrogels: Toward Construction of Floating (Bio)microdevices. Chem. Mater. 2016, 28, 3641-3648.
(43) Yao, X.; Chen, L.; Ju, J.; Li, C.; Tian, Y.; Jiang, L.; Liu, M. Superhydrophobic Diffusion Barriers for Hydrogels via Confined Interfacial Modification. Adv. Mater. 2016, 28, 7383-7389.
(44) Donnelly, P. E.; Jones, C. M.; Bandini, S. B.; Singh, S.; Schwartz, J.; Schwarzbauer, J. E. A simple nanoscale interface directs alignment of a confluent cell layer on oxide and polymer surfaces. J. Mater. Chem. B 2013, 1, 3553-3561.
(45) Harris, G. M.; Madigan, N. N.; Enquist, L. W.; Windebank, A. J.; Schwartz, J.; Schwarzbauer, J. E. Nerve Guidance by a Decellularized Fibroblast Extracellular Matrix. Matrix Biol. 2017, 60-61, 176-189.
(46) Danahy, M. P.; Avaltroni, M. J.; Midwood, K. S.; Schwarzbauer, J. E.; Schwartz, J. Self-assembled Monolayers of α,ω-Diphosphonic Acids on Ti Enable Complete or Spatially Controlled Surface Derivatization. Langmuir 2004, 20, 5333-5337.
(47) Shannon, F. J.; Cottrell, J. M.; Deng, X.-H.; Crowder, K. N.; Doty, S. B.; Avaltroni, M. J.; Warren, R. F.; Wright, T. M.; Schwartz, J. A novel surface treatment for porous metallic implants that improves the rate of bony ongrowth. J. Biomed. Mater. Res., Part A 2008, 86, 857-864.
(48) Taylor, S. E.; Cao, T.; Talauliker, P. M.; Lifshitz, J. Objective Morphological Quantification of Microscopic Images Using a Fast Fourier Transform (FFT) Analysis. Current Protocols Essential Laboratory Techniques; Wiley Online Library, 2013.
(49) Alexander, J. K.; Fuss, B.; Colello, R. J. Electric field-induced astrocyte alignment directs neurite outgrowth. Neuron Glia Biol. 2006, 2, 93-103.
(50) Bandini, S. B.; Spechler, J. A.; Donnelly, P. E.; Lim, K.; Arnold, C. B.; Schwarzbauer, J. E.; Schwartz, J. Perforation Does Not Compromise Patterned Two-Dimensional Substrates for Cell Attach-ment and Aligned Spreading. ACS Biomater. Sci. Eng. 2017, 3, 3123-3127.

The citation of any document or reference is not to be construed as an admission that it is prior art with respect to the present invention.

### Example 3

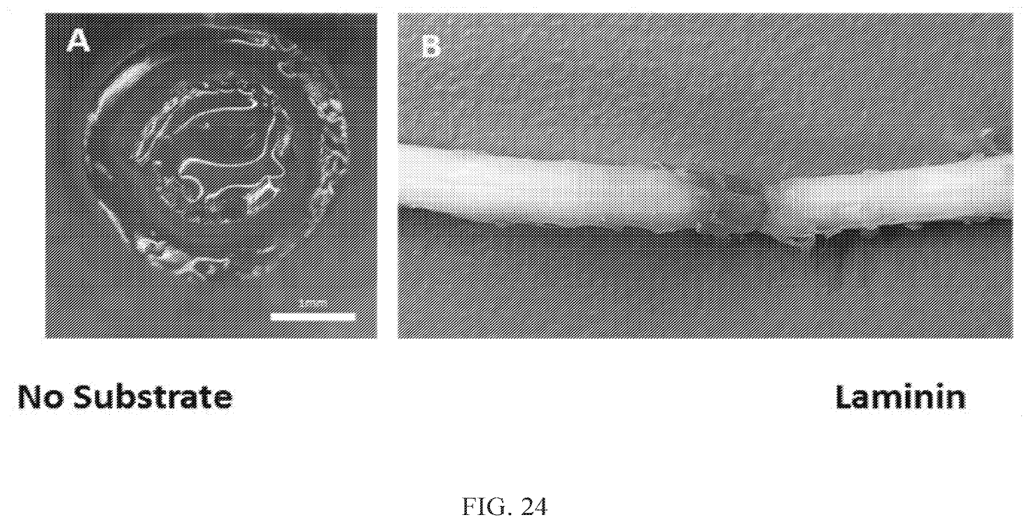

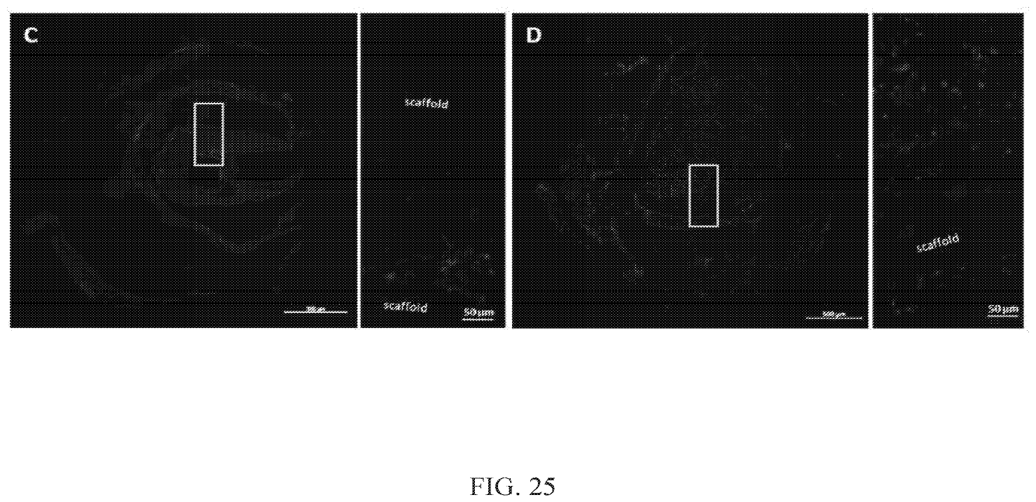

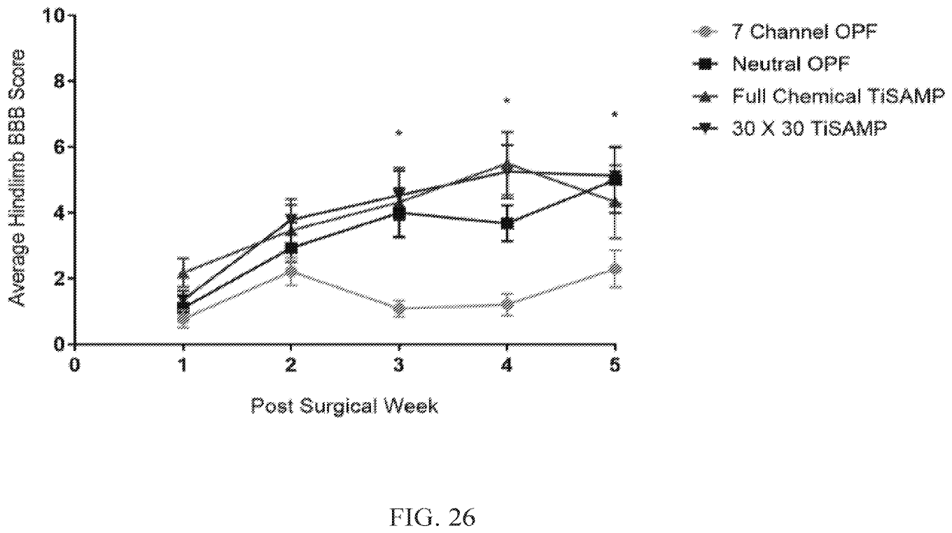

With our initial design we performed a proof of principal experiment in rats where they received a thoracic level 9 (T9) transection followed by placement of our rolled OPF+ scaffold. The OPF+ scaffold contained ridges that were 0.4 mm apart and were coated with either laminin or serum present in the media (control). The scaffold sheet was rolled and placed into an OPF+ ring to hold it securely (FIG. 24). Over a 4 week period, the scaffold integrated well with the spinal cord (FIG. 24). Immunohistochemistry for neurofilament indicated that both scaffolds could support the growth of axons through the scaffold (FIG. 25). Although this pilot study was not powered to determine statistical differences between the conditions, visual inspection and initial counts showed that the laminin coated sheets had greater number of axons than the control. This study demonstrated that this rolled scaffold design constructed of OPF could potentially support axon regeneration.

Subsequently, a group of 24 female Sprague Dawley rats underwent a T9 spinal cord transection and had one of the following three scaffold designs (n=7 per group) implanted at the site of injury: a neutral OPF rolled scaffold, a rolled OPF unpatterned, scaffold fully coated with TiO₂/SAMP to promote cell adhesion or a rolled OPF sheet chemically treated with TiO₂/SAMP in a 30×30 micron stripe pattern to promote directed neurite outgrowth. Animals were followed up for 5 weeks and had a weekly motor function assessment (Basso, Beattie and Bresnahan (BBB) test of hindlimb motor function) performed. Extensive motor recovery was achieved in all three scaffold groups by week 5, most consistently in animals treated with a chemically and physically enhanced design. No statistically significant differences were observed between these groups despite a trend towards an enhanced motor recovery in the patterned and unpatterned TiO₂/SAMP versus neutral OPF alone. However, differences were observed between the mean hindlimb function of all three rolled OPF scaffold groups when compared to the more traditional 7-channel (n=11) OPF+ scaffold loaded with Matrigel employed by our group. Statistical significance was achieved at weeks 3, 4 and 5 weeks post spinal cord injury surgery compared to all groups (2 way ANOVA with Tukeys multiple comparisons * p<0.05).

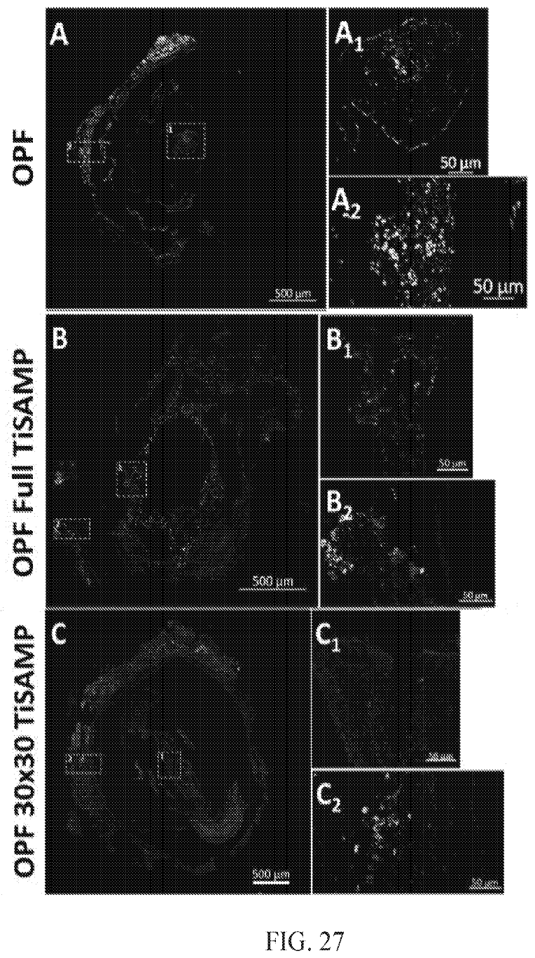

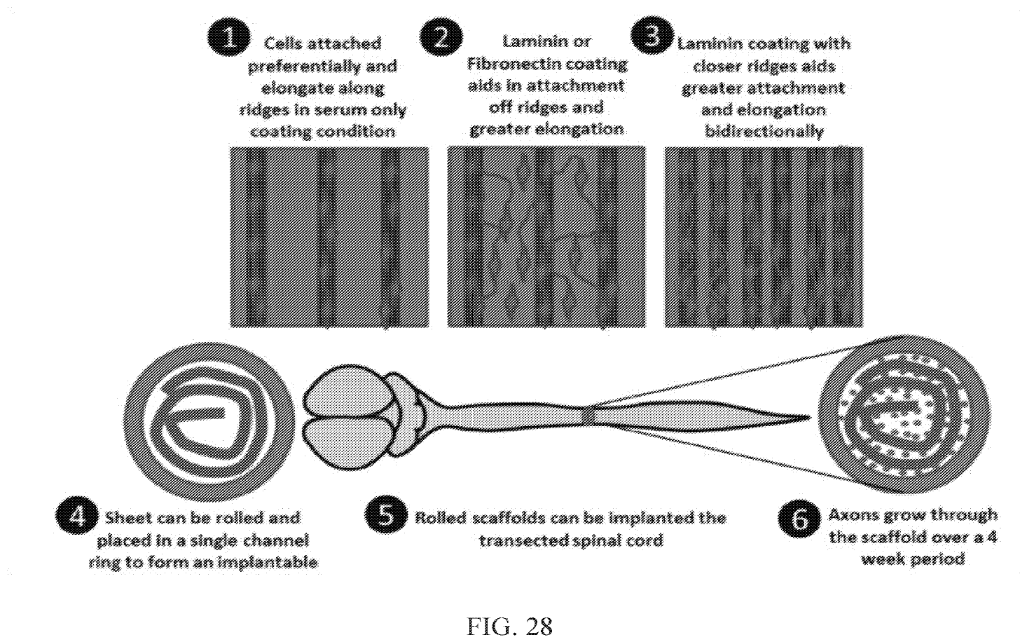

As demonstrated in FIG. 24, this scaffold design intergrades with the spinal cord. Immunohistochemistry of transverse 10 μm section of the intergraded scaffold was done for axons (with β-tubulin; red) and myelin (with myelin basic protein; cyan). Layers of the spiral shape are evident as concentric rings. Axon are seen growing through the enwrapping of the scaffold sheet (FIG. 27 A1, B1, C1) as well as the outside surface (FIG. 27 A2, B2, C2). Some myelinate axons are seen inside the scaffold (FIG. 27 A1, B1, C1) but more are present in the more outer layers (FIG. 27 A2, B2, C2).

### Example 4

Spinal cord injury (SCI) results in cell death, demyelination, and axonal loss. The spinal cord has a limited ability to regenerate and current clinical therapies for SCI are not effective in helping promote neurologic recovery. We have developed a novel scaffold biomaterial that is fabricated from the biodegradable hydrogel oligo[poly(ethylene glycol)fumarate] (OPF). We have previously shown that positively charged OPF scaffolds (OPF+) in an open spaced, multichannel design can be loaded with Schwann cells to support axonal generation and functional recovery following SCI. We have now developed a hybrid OPF+ biomaterial that increases the surface area available for cell attachment and that contains an aligned microarchitecture and extracellular matrix (ECM) proteins to better support axonal regeneration. OPF+ was fabricated as 0.08 mm thick sheets containing 100 μm high polymer ridges that self-assembles into a spiral shape when hydrated. Laminin, fibronectin, or collagen I coating promoted neuron attachment and axonal outgrowth on the scaffold surface. In addition, the ridges aligned axons in a longitudinal bipolar orientation. Decreasing the space between the ridges increased the number of cells and neurites aligned in the direction of the ridge. Schwann cells seeded on laminin coated OPF+ sheets aligned along the ridges over a 6-day period and could myelinate dorsal root ganglion neurons over 4 weeks. The OPF+ sheets support axonal regeneration when implanted into the transected spinal cord. This novel scaffold design, with closer spaced ridges and Schwann cells is a novel biomaterial construct to promote regeneration after SCI.

The spinal cord has a limited ability to regenerate after spinal cord injury (SCI) and available therapies are not efficacious in promoting recovery of motor and sensory neurological function. The failure to recover function may be due to secondary events that occur after the primary insult such as cell death, axonal loss, demyelination, cyst formation and an increase in the inhibitory microenvironment [1]. Many therapies are currently under investigation for treating one or more of these secondary events using neuroprotective strategies and cell, regenerative, and rehabilitative therapies [1, 2]. A single therapeutic strategy is unlikely to be effective in treating a disorder that is heterogeneous in its underlying pathophysiology. Combinatorial treatments that simultaneously address multiple contributions to the residual of the injury will be required.

Biomaterial scaffolds are an attractive platform for combinatorial therapies because they are able to bridge the physical gap produced from gliosis, cyst formation, and cell death by providing structural support for regenerating cells [3, 4]. Biomaterials can be combined with extracellular matrices, cells, or pharmaceuticals to promote a more hospitable environment for regeneration [3, 5, 6]. We have developed a positively charged polymer, oligo[poly(ethylene glycol)fumarate] (OPF+) for scaffold fabrication in a multichannel design that overlays the different tracts of the spinal cord [4, 7]. OPF+ forms a porous, biodegradable hydrogel that is mechanically similar to the spinal cord [8]. The positive charge on OPF enhanced neuronal cell attachment, extension and the axonal myelination with Schwann cells in vitro [9]. In vivo, OPF+ loaded with Matrigel supported axonal regeneration over 4 weeks [10]. OPF+ scaffolds loaded with Schwann cells further improved axonal regeneration, and growth orientation over that seen with Matrigel alone, and reduced collagen scarring, cyst formation, and proteoglycan accumulation following transection injury [4, 7, 10]. Schwann cells, that were genetically modified to express high concentrations of glial cell-derived neurotrophic factor (GDNF-SC), increased axonal regeneration and myelination compared to wild type Schwann cells and led to enhanced functional recovery following spinal cord transection [5].

Axonal regeneration occurred through the open channels in the scaffold, as well as on the outer surface of the scaffolds. This observation led to the new hypothesis that changing the scaffold architecture to increase the surface area available for axons to regenerate may increase the number of axons regenerating through the scaffold and provide directional guidance. Different scaffold designs have been fabricated with a higher proportion of open to closed spaces in association with a novel ridged surface architecture that would guide regenerating axons. Physical structures for contact mediated axonal guidance, such as microgrooves or ridges, have been created by laser etching onto polymer surfaces [11-13]. Initial studies using quartz slides microgrooves improved the directional orientation of seeded fibroblast and epithelial cells but did not align chick embryo neurons [11]. Yao et al. (2009) subsequently demonstrated that micro-patterned poly(lactide-co-glycolide) (PLGA) sheets increased neurite alignment. In another study, neonatal rat dorsal root ganglion (DRG) neurons were cultured on grooved polymers of poly(dimethyl siloxane) coated with poly-L-lysine and laminin. On this surface neurites extended along and arched over the grooves with the somas preferentially adhering to the ridges [14].

The addition of extracellular matrix (ECM) proteins onto polymer surfaces provides sites for receptor-mediated cell attachment to promote axonal guidance and cell migration [15]. Grooved polymers coated with gradients of laminin or collagen have been shown to direct axonal growth cones [16, 17]. Fibronectin promotes Schwann cell proliferation and motility in culture [18], and is an attractive ECM candidate to make biomaterials more cell adhesive and facilitate growth. Peripheral nerve regeneration in rats can be enhanced by the addition of fibronectin and Schwann cells in alginate hydrogels [19]. Fibronectin has also been shown to help increase serotonergic axon sprouting and can influence axonal growth when used in combination with cells, growth factors, and biomaterials [20, 21].

In this study we sought to determine which ECM molecules best support neuronal attachment, and whether the structural microarchitecture may further improve the directional alignment of axonal extension and myelination in vitro. A novel hybrid OPF+ scaffold design was fabricated as a ridged, flat sheet and coated with ECM proteins. Upon hydration the sheets had the unique property of spontaneously rolling into a spiral 3D configuration. Polymer sheets with ridge spacings of 0.2 mm, 0.4 mm, or 1 mm apart were coated with laminin, collagen, or fibronectin, and used in in vitro assays to quantitate neuronal attachment, DRG neurite extension, and axonal myelination by Schwann cells. A proof of concept study was then conducted in vivo to compare axonal regeneration in laminin and untreated spiral scaffolds implanted following complete spinal cord transection.

OPF+ was synthesized as described previously [8, 9]. To create the ridged OPF+ sheet, liquid polymer was pipetted onto a Teflon mold that contained micro-grooves 100 μm in depth and spaced 1 mm, 0.4 mm and 0.2 mm apart. A glass slide was place on top with a 0.08 mm thick spacer. The sheets were polymerized by exposure to an ultra-violet (UV) light (365 nm) for one hour and cured overnight. Single channel OPF+ scaffolds were fabricated by mold injection of the polymer, and cast over a 1 mm wire prior to UV exposure. The scaffolds were cut into 2 mm lengths for transplantation.

Desiccated, ridged OPF+ sheets were cut into 6×6 mm pieces and weighed dry (W_{d}) and following hydration in distilled water for 24 hours (Wₛ; swollen weight). The swelling ratio was calculated using the equation:

Swelling ratio=(Wₛ−W_{d})/W_{d}

OPF+ sheets were sterilized by immersion in serial dilution of ethanol, rehydrated in cell culture media and then pinned flat onto sterile culture dishes containing a layer of silicone elastomer (SYLGARD® 184 Silicone Elastomer Kit; Dow Corning, Midland, Mich., USA). The OPF+ sheets containing ridges spaced 1 mm apart were coated with rat tail collagen 1 (3.36 mg/ml; Corning®, New York, USA), laminin (100 μg/ml; Sigma-Aldrich®, St. Louis, Mo., USA), or rat plasma fibronectin (10 μg/ml) overnight at 37° C. Control sheets received no additional ECM protein coating other than that which would be present in the serum of the media (referred to as serum coated). The fibronectin was purified by gelatin-Sepharose affinity chromatography from frozen rat plasma [6]. The sheets were then washed 3 times to remove unabsorbed ECM proteins. To determine the effect of dissociated DRG attachment and alignment on OPF+ sheets with different ridge sizes, OPF+ sheets with ridges 0.2 mm, 0.4 mm, and 1 mm apart were coated with laminin.

DRG neurons were isolated from Sprague Dawley pups on embryonic day 15 as previously described [22]. Whole DRG were placed on the scaffold in media containing modified Eagle's medium (MEM; Gibco-BRL, Gaithersburg, Md.) supplemented with 10% calf bovine serum (CBS) (HyClone, Logan, Utah, USA), L-glutamine (1.2 mM; Gibco), glucose (7 mg/ml; Sigma Aldrich) and nerve growth factor (NGF, 5 ng/ml; Harlan Bioproducts, Indianapolis, Ind., USA). Initially, the explants were incubated for 1 hour at 37° C. in 150 μL media to allow them time to attach. Once the DRG explants were attached the plate was filled with media.

Eighteen DRGs for each condition were plated with 4-6 DRGs per scaffold. DRG explants were imaged at 24 and 48 hours after isolation using a Zeiss Axiovert Model 35 microscope with a Nikon CCD camera. Length of the longest neurite of each DRG was analyzed by measuring from the edge of the DRG to the end of the longest outgrowing neurite using Image J (NIH). Given a variable degree of DRG attachment on each type of substrate, the final numbers of explants available for analysis was n=17 DRGs for laminin, n=11 DRGs for collagen, n=11 DRGs for fibronectin, and n=4 DRGs for serum coated.

Dissociated DRG neurons were isolated from Sprague Dawley pups on embryonic day 15 as previously described [22]. Approximately 100-200 whole DRGs were pooled and dissociated using trypsin and mechanical trituration. 50,000 cells were plated onto each scaffold sheet, and were grown in MEM supplemented with 15% CBS (HyClone, Logan, Utah, USA), L-glutamine (1.2 mM; Gibco), glucose (7 mg/ml; Sigma Aldrich) and NGF (5 ng/ml; Harlan Bioproducts, Indianapolis, Ind., USA). Dissociated cultures were treated for 3 days with 1×10⁻⁵ M 5-fluoro-2-deoxy-uridine and with 1×10⁻⁵ M uridine (Sigma) to remove non-neuronal cells[23, 24]. The cells were fixed with 4% paraformaldehyde for 30 minutes and stored in phosphate buffered saline (PBS) at 4° C. for immunocytochemistry.

The fixed OPF+ sheets were blocked with 10% normal donkey serum and 0.1% Triton X-100 in 0.01M PBS for 30 minutes. Primary antibodies against mouse anti-β-III tubulin (1:300, Millipore) and rabbit anti-myelin basic protein (1:800, Abcam) were diluted in PBS containing 5% normal donkey serum and 0.3% Triton X-100 at 4° C. overnight. Secondary antibodies (donkey anti-mouse Cy3; Millpore; 1:200 and donkey anti-rabbit Alexa 647; Jackson ImmunoResearch Laboratories; 1:200) were diluted with 5% normal donkey serum and 0.3% Triton-X-100 in PBS. After removing the pins, the OPF+ sheets were carefully placed onto a glass slide using forceps with the ridges facing upwards. Glass coverslips were mounted using Slow Fade Gold Antifade Reagent with DAPI nuclear stain (Molecular Probes®, Eugene, Oreg., USA).

All images of dissociated neurons were taken using an inverted fluorescence microscope Zeiss Axio Observer Z-1 with a motorized stage (Carl Zeiss, Inc., Oberkochen, Germany) mounted with an Axiocam 503 camera (Zeiss). Pictures were acquired via the ZEN 2 (blue edition) imaging software (Carl Zeiss). For cell quantification, z-stacks with approximately 5 slices at magnification of 10× were taken using an EC Plan-Neofluar 10×/0.30 Ph1 objective. On each polymer sheet, 4 representative areas of different ridges and the adjacent flat parts were systematically captured. To avoid bias, the regions were chosen by moving one field of view up or down form the centered position and then 2 fields across left or right to yield a total of 4 quadrants. The DRG neurons preparation was repeated at least 3 times. Cell nuclei (DAPI), which were associated with axons (β-tubulin, red) were counted in each image in an area of 878.94 μm×662.84 μm on the polymer to quantify neuronal cell attachment.

In order to determine the ECM protein-coating that displayed most neurite outgrowth and attachment, the amount of β-III tubulin was calculated for each polymer sheet using ImageJ software by measuring the mean gray value of the staining. The amount of β-tubulin staining was normalized to the amount of DAPI (mean grey value) per cell. A mean value of normalized β-tubulin mean gray values was derived for each polymer sheet from replicates of the 4 fields of views. For comparison between different ECM molecules on OPF+ sheets with 1 mm spaced ridges, laminin coated sheets had n=7 sheets, fibronectin coated sheets had n=3 sheets, collagen coated sheets had n=4 sheet, and sheets with no substrate had n=6 sheets. For different spaced ridges, OPF+ sheets with 0.2 mm spaced ridges had n=3 sheets, 0.4 mm spaced ridged sheets had n=2 sheets, and 1 mm ridged sheets had n=4 sheets.

Primary rat Schwann cells were isolated from sciatic nerves of 2-5 day old Sprague Dawley pups as previously described [25, 26]. 125,000 cells were seeded on to laminin coated OPF+ ridged sheets. Schwann cells were imaged using Zeiss Axiovert Model 35 microscope with a Nikon CCD camera at 24 hours and 6 days. For each condition 3 scaffold replicates were made and images were taken of 4 fields of views per scaffold.

Schwann cells and dissociated DRG neurons were isolated and plated together (1:1 ratio, 100,000 cells in total per scaffold) on OPF+ ridged sheets. The cells were cultured for 4 weeks in MEM supplemented with 15% CBS (HyClone, Logan, Utah, USA), L-glutamine (1.2 mM; Gibco), glucose (7 mg/ml; Sigma Aldrich) and NGF (5 ng/ml; Harlan Bioproducts, Indianapolis, Ind., USA), 50 μg/mL of ascorbic acid (Sigma) and 2 μM Forskolin (Sigma). For each condition 3 scaffold replicates were made and images were taken of 4 fields of views per scaffold using a Zeiss LSM780 confocal microscope system.

OPF+ sheets and single channel scaffolds were sterilized with serial dilution of ethanol. 2 mm by 6 mm OPF+ sheets with ridges 0.4 mm apart were either coated with laminin (n=1; 100 μg/ml) or had only serum coating (n=1). The scaffold sheet was rolled along the long edged and placed inside the single channel scaffold with 2 mm length. Female Sprague Dawley rats (230-300 grams; Harlan Laboratories, Madison, Wis.) received a laminectomy at level T8-T10 and a complete spinal cord transection was performed at level T9. The scaffold was placed into the transected area and all the muscles were closed with sutures. The animals were cared for by the research team and by veterinarians their staff experienced with care of rats with spinal cord injury daily. All procedures were approved by the Mayo Clinic Institutional Animal Care and Use Committee and all guidelines were followed in accordance with the National Institute of Health, Institute for Laboratory Animal Research and the United States Public Health Services Policy on the Humane Care and Use of Laboratory Animals.

The animals were euthanized 4 weeks after implantation and injury by deep anesthesia and fixed by transcardial perfusion with 4% paraformaldehyde in PBS. The spinal column was removed en bloc and post fixed for 2 day at 4° C. The spinal cords were dissected out and a 1 cm segment with scaffold (T9) in the center was embedded in paraffin. Transverse sections 8 μm thick were made on a Reichert-Jung Biocut microtome (Leica, Bannockburn, Ill.). Tissue sections were deparaffinized and immunostained as we have described (Chen et al 2017) using a mouse anti-human neurofilament primary antibody (1:100; DAKO) and a donkey anti-mouse IgG secondary antibody conjugated to Cy3.

The sections were imaged using Zeiss Axiovert Model 35 microscope with a Nikon CCD camera. The microscope contained a motorized stage and the tiling feature was used in the Zen Blue (Zeiss) software to image the whole scaffold section. Neurofilament staining identified the axons running through the scaffold.

All data are reported as mean±standard error of the mean (SEM). All statistics were calculated using GraphPad Prism 7 (GraphPad Software, Inc., USA). Statistical significance was calculated using one-way ANOVA with multiple comparisons using Tukey's post-hoc testing (unless stated otherwise). On graphs p-values are represented as *p<0.05, ** p<0.01, *** p<0.001, **** p<0.0001.

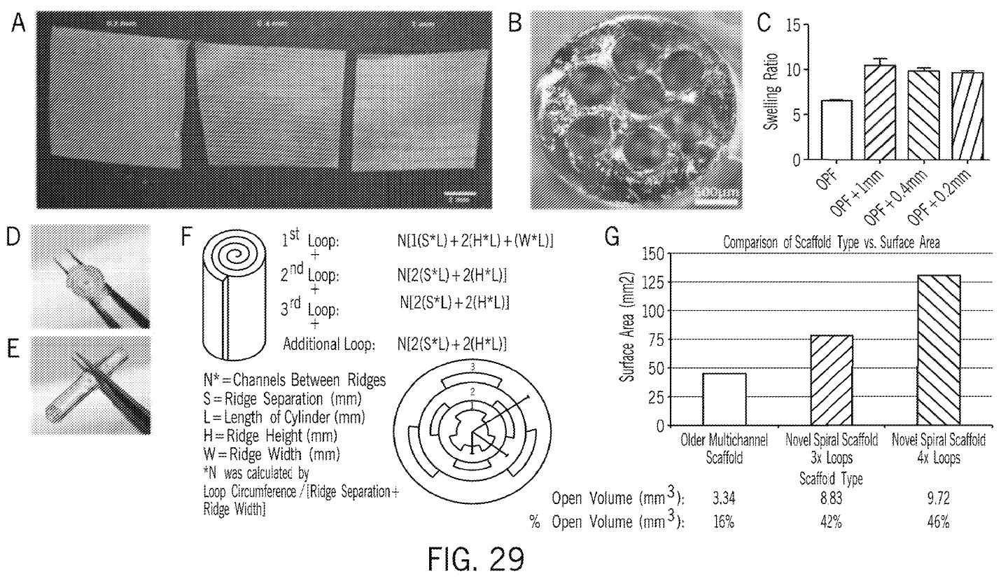

0.08 mm thick OPF+ sheets were embedded with longitudinal ridges spaced 0.2 mm, 0.4 mm, or 1 mm apart (FIG. 29A). Our previously used scaffold was fabricated with 7 channels that were 2 mm wide and 32 mm long (FIG. 29B). Swelling ratios were calculated to be 10.5±0.8 for OPF+ sheets with 1 mm spaced ridges, 9.8±0.4 for sheets with 0.4 mm spaced ridges, and 9.7±0.1 for sheets with 0.2 mm spaced ridges (FIG. 29C). Hydrated sheets spontaneously rolled into a spiral configuration (FIGS. 29D and 29E). In comparison to a multichannel scaffold (FIG. 29B), the rolled-sheet design allowed for a greater volume of open space inside of the scaffold, and increased surface area that was dependent on the number of loops in the spiral (FIGS. 29F and 29G). Internal volumes and surface areas were calculated by taking into account the number of channels that form between ridges, ridge separation (mm), length of the cylinder (mm), and ridge width (mm) (FIG. 29F). A scaffold sheet with 3× loops increased the volume by 2.6 fold (3.34 mm³ to 8.83 mm³), whereas a sheet with 4× loops has an increase of approximately 3 fold (3.34 mm³ to 9.72 mm³) compared to the 7 multichannel OPF+ scaffold design (FIG. 29F). There was a similar corresponding increase in surface area where sheets with 3× loops had 1.71 fold increase (45.4 mm² to 77.7 mm²) and 4× loops had 2.87 fold increase (45.4 mm² to 130.5 mm²) compared to the 7 channel design.

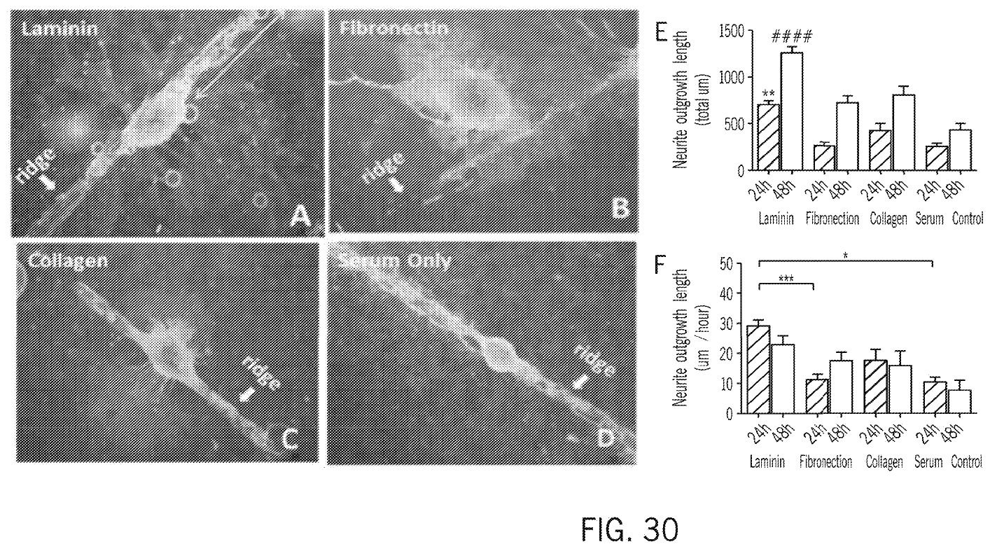

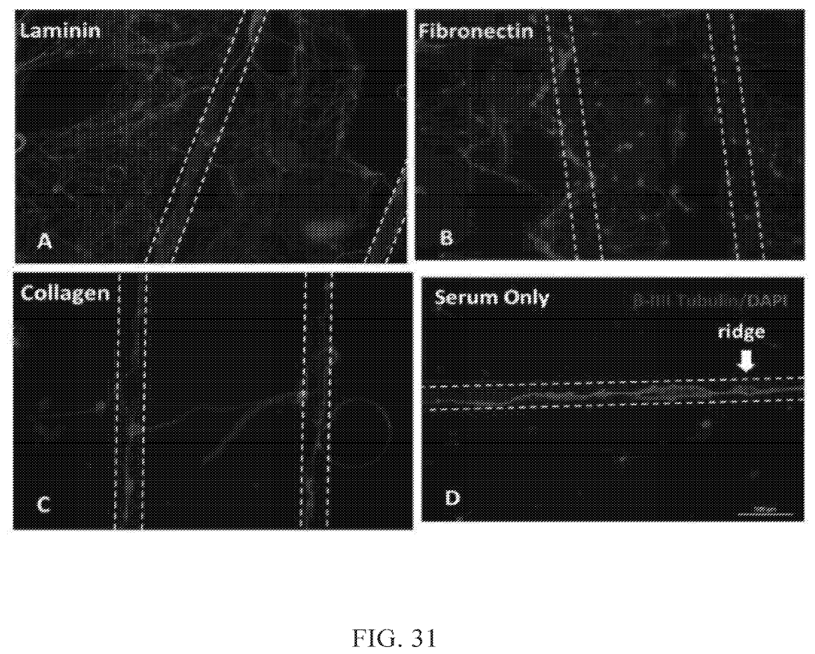

Whole DRGs were used to determine the effect of different ECM protein coatings (laminin, fibronectin, and collagen) on promoting neurite outgrowth on OPF+ sheets with ridges 1 mm apart. The explants were observed to consistently settle on or immediately adjacent to a ridge, rather than on the broader, flat intervening surfaces (FIG. 30). The DRGs elongated along the ridge, and into the adjacent areas in conditions where ECM protein coatings were present in contrast to OPF+ sheets with serum coating. Within 24 hours of culture, laminin-coated OPF+ sheets had longer neurite outgrowth (708.5±34.93 μm, FIG. 30E) than controls with serum coating only (255.7±37.12 μm, p=0.004), fibronectin (288.3±33.72 μm, p<0.0001) and collagen (433.6±74.56 μm, p=0.0362). Outgrowth from collagen and fibronectin was not significantly different from serum coated control surfaces. After 48 hours of culture, laminin coated OPF+ sheets (1261±58.98 p<0.0001) continued to support greater neurite outgrowth than serum coated control (445.1±61.15 μm), fibronectin (731.9±69.99 μm, p<0.0001), and collagen (817.3±89.9 μm, p<0.0001), with collagen and fibronectin not significantly different to serum coated control.

The outgrowth rate (μm/h) of whole DRG neurites over 48 hours in culture between the different ECM coatings (FIG. 30F) was compared. In the first 24 hours laminin (29.52±1.45 μm/h, p=0.017) enabled a higher rate of neurite outgrowth than was observed on OPF+ sheets with serum coating (10.66±1.55 μm/h). There was no significant difference between the outgrowth rates of fibronectin (11.64±1.46 μm; p=0.9977) and collagen (18.07±3.11 μm/h; p=0.9065) compared to serum coating. In the next 24 hours there were no significant differences between laminin (23.03±2.85 μm/h; p=0.1120), fibronectin (17.64±2.8 μm/h; p=0.6721), and collagen (15.99±4.87 μm/h; p=0.8394) as compared with serum coating (7.88±2.98 μm/h).

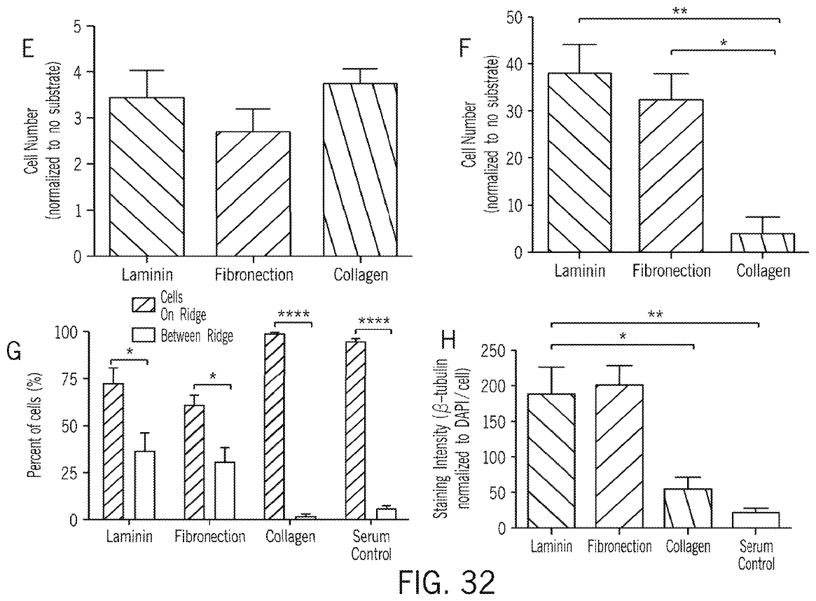

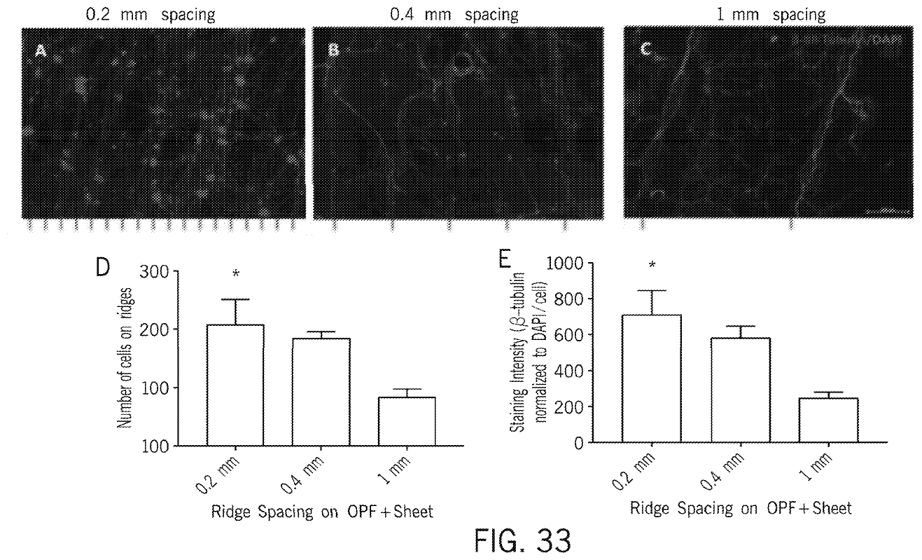

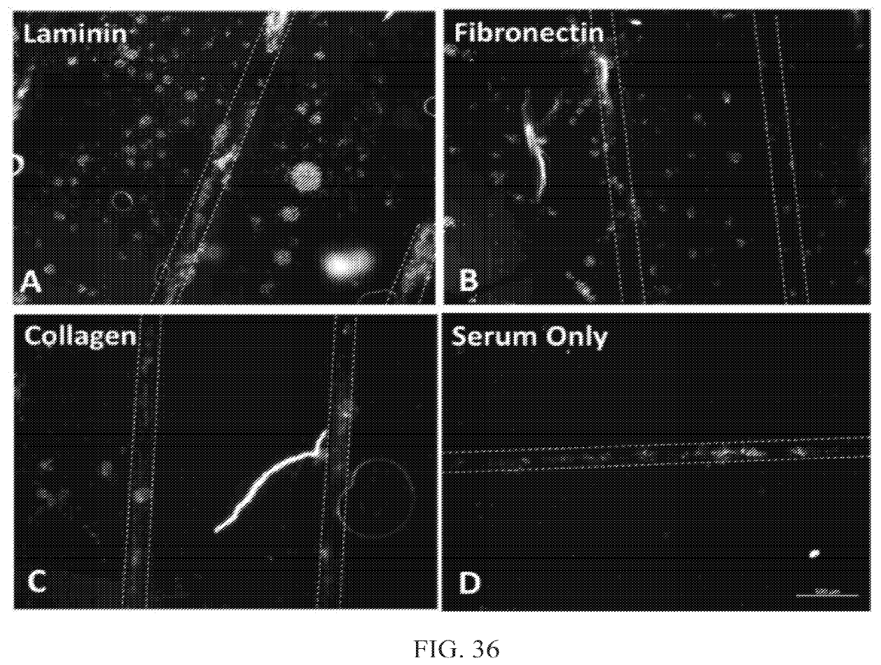

OPF+ sheets with ridges 1 mm apart and laminin, fibronectin or collagen ECM supported dispersed neuronal cell attachment and outgrowth. On all surfaces, dissociated neurons preferentially attached to the ridges, and aligned their axons longitudinally along the ridge (FIG. 31, FIG. 32, FIG. 36). The use of laminin, fibronectin, or collagen increased the number of adherent neurons on the ridge by 3.4±0.62 fold, 2.69±0.50 fold, and 3.7±0.32 fold, respectively over ridged sheets with serum coating (FIG. 31E). The use of laminin resulted in a greater increase in neuronal attachment (38.1±5.98 fold) in the spaces between the ridges compared to collagen coating (4.25±3.3 fold) (p=0.003; FIG. 3F). Neuronal attachment in between the ridges on fibronectin-coated sheets (32.36±5.65 fold, p=0.013) was also greater statistically than those attached on collagen-coated sheets. 71.67±10.76% (p=0.0127) of neurons attached to the ridges on laminin coated sheets, 60.33±10.67% (p=0.0374) on fibronectin coated sheets, 98.33±12.32% (p<0.0001) on collagen coated sheets, and 94.33±12.32% (p<0.0001; 2-way ANOVA/Sidak's multiple comparison) on serum coated sheets, demonstrating a preferential neuronal attachment onto the ridge (FIG. 32G).

The neurite density of dissociated neurons was also greater on laminin and fibronectin coated sheets (FIG. 32H), as measured by stereologic analysis of the areas of β-tubulin staining. Laminin coated sheets (185.3±41.02 mean gray value; p=0.002) and fibronectin (198.4±30.14 mean gray value; p=0.0408) had higher total β-tubulin staining per cell than sheets with serum coating (20.73±5.59 mean gray value). The neurite density on laminin coated sheets (p=0.0263) and fibronectin coated sheets (p=0.0408) was also greater than that seen on collagen coated sheets.

To determine what effect ridge spacing distance may have on neuronal cell attachment and alignment, laminin-coated OPF+ sheets with ridges 0.2 mm, 0.4 mm, and 1 mm apart were used. Tighter ridge spacing increased the number of ridges per area and the overall surface area of the sheet, (FIG. 33 A-C) which in turn improved neuronal cell attachment. Many of the cells settled around or on top of the ridges, extending neurites along the ridge. The number of neuronal cells located on the ridge surface was 2.5 fold greater for the 0.2 mm spaced OPF+ sheets (FIG. 33D; 208.6±44.3 cells; p=0.039) than the 1 mm OPF+ sheets (84.8±15.1 cells). The 0.4 mm OPF+ sheets (FIG. 33D; 186.4±11.9 cells; p=0.1215) had 2 fold greater number of cells than the 1 mm spaced OPF+ sheets. The neurite density was increased on the OPF+ sheets with ridges more closely spaced (0.2 mm and 0.4 mm apart). We found a 2.9 fold increase in the amount of β-III tubulin staining (normalized to the amount of DAPI staining/cell) when comparing OPF+ sheets with 0.2 mm spaced ridges (FIG. 4 E; 719.5±130.3 mean grey value; p=0.012) to OPF+ sheets with 1 mm spaced ridges (251.2±33.4 mean grey value).

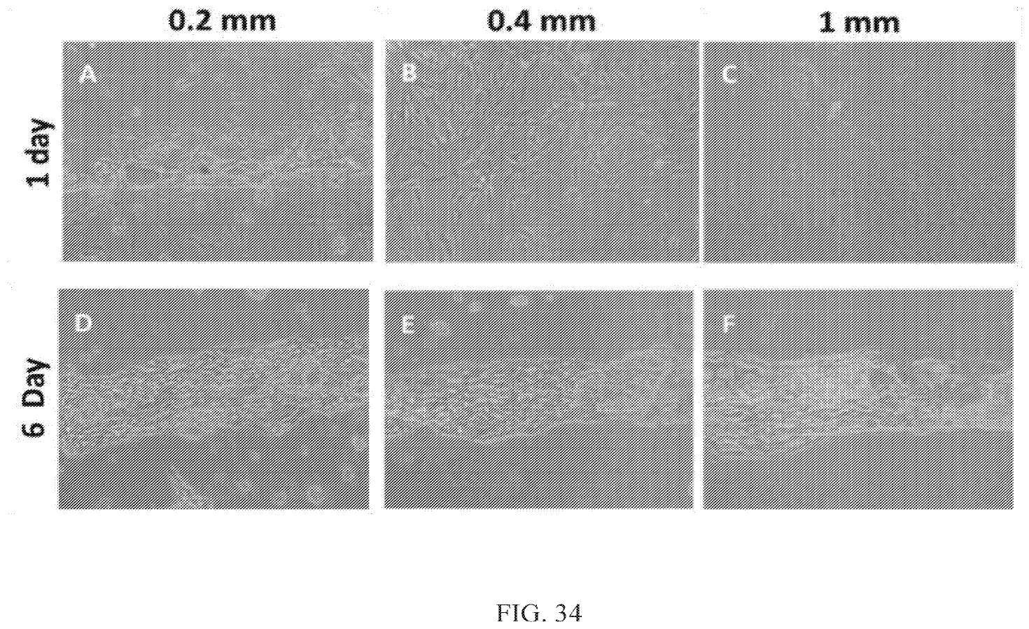

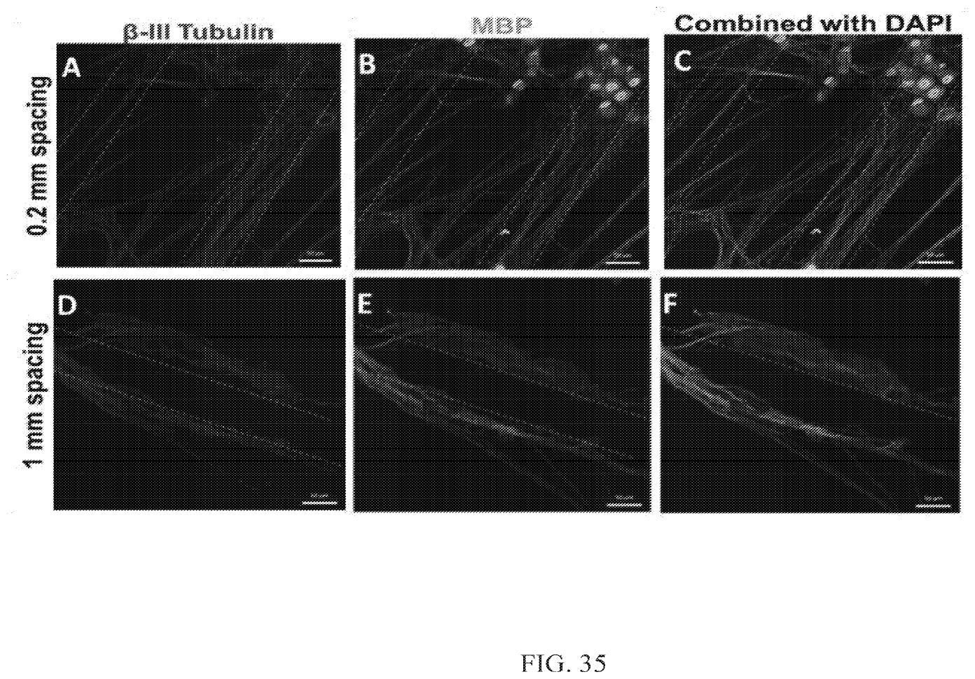

In our previous studies we have loaded the multichannel scaffolds with genetically modified Schwann cells which facilitated axonal regeneration and recovery after spinal cord transection [5]. We next sought to demonstrate that the OPF+ ridged sheet design with laminin coating would support the growth of Schwann cells. Schwann cells on OPF+ ridged sheets initially formed a cell layer in a dispersed and unorganized manner (FIG. 34 A-C). Over a 6 day period the Schwann cells organized along the ridges of the scaffold (FIG. 34 D-F). When the Schwann cells were co-cultured with dispersed DRG neurons for 4 weeks, neurites became myelinated (FIG. 35). Neurites aligned well with the ridges and stained positively for myelin basic protein (MBP). Greater densities of aligned myelinated neurites were observed when the ridges were spaced closer together (0.2 mm spacing vs. 1 mm spacing).

A proof of principle pilot experiment was conducted (see, e.g., example 3) in which uncoated and laminin-coated OPF+ sheets with ridges 0.4 mm apart, were rolled and secured inside a single channel OPF+ scaffold. This scaffold was then implanted into the transected rat spinal cord at the T9 level. The scaffold integrated well with the spinal cord over a 4 week period. Staining for β-III tubulin indicates that both scaffolds promoted axon outgrowth into the scaffold. Although the number of animals was not powered to determine statistical significance (n=1 per condition), these preliminary results show the potential of our novel rolled scaffold for spinal cord regeneration.

We have previously demonstrated that OPF+ fabricated in a multichannel design with seven channels can enhance regeneration after SCI [4, 7] in rats. This regeneration can be further improved by loading the channels with GDNF-secreting Schwann cells, leading to modest functional recovery [5]. We have also shown that the OPF+ material can be modified by incorporating the anti-fibrotic drug rapamycin in PLGA microspheres [27]. OPF+ multichannel scaffolds containing rapamycin microspheres were also combined with Schwann cells to help promote functional recovery following SCI. It was also noted from these studies that many of the regenerating axons grew on the outer circumferential surface of the scaffold. We therefore hypothesized that axonal growth may preferentially occur on surfaces rather than within open spaces. Maximizing the surface areas available to regenerating axons to extend may further improve the density and directionality of regrowth, and impact upon functional reconnections. In this study, we have now demonstrated, in vitro, that OPF+ fabricated in a flat sheet design with ridges increased the surface area available for growth when compared to the multichannel design scaffold. Providing an attractive ECM protein coat and spacing the ridges closer together improved cell attachment, outgrowth, and alignment of DRG explants, neurons, and Schwann cells. We also demonstrated that this scaffold design is feasible to use in vivo.

When hydrated, the OPF+ sheets spontaneously form a 3D spiral tube. Hydrogels have been designed to self-roll in previous studies either by introducing reductants to cleave disulfide bonds [28], changes in pH and sacrificial layers [29], or using light responsive inks [30] and photolithography [31]. These ridged scaffolds had swelling ratios comparable to our previous studies [32]. Hydrogels may suffer from low cell attachment due to the hydrated surface layer [33]. This issue could be addressed by coating the hydrogel sheets with collagen, laminin, or fibronectin, all of which are classically established as ECM coatings used to promote neurite outgrowth [6, 13, 34, 35]. The current study shows that DRG explants cultured on laminin coated OPF+ sheets had longer neurite outgrowth than fibronectin or collagen coated sheets. In addition, the outgrowth rate of neurites on laminin coated sheets was faster in the first 24 hours of culture. This can be due to the different amount of proteins used or absorbed into the scaffold. Previous studies have demonstrated concentration dependent differences in cell attachment and outgrowth, however, concentrations used in this study were within range of those used in other studies [6, 24, 36, 37]. It is also interesting to note that the DRG outgrowth rate on the fibronectin coated sheets was still increasing at the 24-48 hour time point and may have results in greater outgrowth length at a later time point. When neuronal attachment was measured on or in between the ridges, laminin and fibronectin did equally well. The observed enhancement of neuronal outgrowth and guidance associated with laminin is in keeping with several other studies investigating different substrates for neuronal regeneration [6, 13, 35, 38]. ECM proteins or their fragments play an important role in cell adhesion through ligand receptor interactions, and these interactions may be an important component to consider in developing novel biomaterials [39-41]. In addition, the three dimensional structure of ECMs help anchor cells and provide axonal guidance [15]. We show that to maximize the ability of hydrogel scaffolds to anchor neurons and their axons and aid in their outgrowth, ECMs like laminin and fibronectin are necessary to maximize the regenerative capability of the scaffold.

The combination of a scaffold containing ridges and ECM may mimic what is seen in development where topographical and chemical cues help guide axons. Factors such as stiffness of the substrate can be important in determining the growth patterns of neurons, as shown by the growth of retinal ganglion cell axons towards softer tissue [42]. Laminin and β-integrin signaling have been found to be important in neuronal polarization and the deletion of β-integrin results in deficits in axonal development [43]. Radial glia, similar to the ridges of the scaffold, provide topographical cues that orientate the migration of embryonic neurons [44]. Neurite outgrowth from DRG explants and disassociated DRG neurons could be directed in a bidirectional way on ridged surfaces in this study. Ridges may provide important topographical cues, as the majority of neurons preferentially attached on or near the ridges of the scaffold. A similar preference for mechanical guidance cues (microgrooves), rather than on flat spaces between the microgrooves, has previously been observed [13]. Studies investigating the effect of grooves on a polymer surface have shown that axonal alignment and behavior could be influenced by the depth of the grooves, spacing between the grooves, and groove widths [13, 38]. Similarly, in our study, disassociated DRG neurons that grew on the OPF+ sheets with ridges spaced 0.2 mm apart had greater cell attachment to the ridges, alignment, and outgrowth than those grown on 1 mm spaced sheets. One of the goals of the scaffold is to bridge the gap that is created after spinal cord injury. The ability of the scaffold to direct growth in a longitudinal direction and maximize this directed growth is an important consideration.

Another approach to enhancing regeneration is to mimic the peripheral nervous system environment. It is well established that the peripheral nervous system has a better ability to regenerate than the central nervous system. This may be due to a more rapid clearance and recycling of myelin debris, more permissive ECM, and a neurotrophic Schwann cell response [45]. The use of Schwann cells with biomaterials recreates a peripheral nervous system environment in the spinal cord, where the cells can contribute regenerative ECM, growth factors, and facilitate remyelination. Our lab, among others, has compared different cell types. Schwann cells have been found to be best able to support axonal regeneration [10, 26, 46, 47]. In this study, Schwann cells preferentially organized along the ridges of OPF+ sheets and myelinated neurites. The OPF+ sheet with ridges spaced 0.2 mm apart had more aligned myelinated neurites observable per area than the 1 mm sheet. This combination of the OPF+ ridged sheet with an ECM and Schwann cells may increase the regenerative potential of the scaffold and these in vitro results suggest that this combination would be helpful in guiding and myelinating aligned axons.

This study demonstrates a novel hybrid design of the OPF+ scaffold for use in regenerative medicine, particularly suited for spinal cord injury. It has properties similar to the spinal cord, maximizes available surface area and volume for regeneration, and can spontaneously roll to make a 3D structure for transplantation. Flattened, ridged sheets were used for in vitro studies and demonstrated improved neuron attachment, axonal alignment, and outgrowth. Furthermore, it would be straightforward to manufacture and scale for large animal studies and clinical applications. Scaffold properties can be modified including varying the ridge distance and size, as well as, coating with different substances. It is also possible to study different cell types for different applications in regenerative medicine. In a small study when implanted into transected spinal cord, the rolled scaffold integrated well and supported regenerating axons. This scaffold is well positioned to be used for combinatorial treatments to treat spinal cord injury through use of novel biomaterials, ECMs, and cells.

### REFERENCES FOR EXAMPLE 4

[1] A. M. Siddiqui, M. Khazaei, M. G. Fehlings, Translating mechanisms of neuroprotection, regeneration, and repair to treatment of spinal cord injury, Progress in brain research 218 (2015) 15-54.
[2] A. Badner, A. M. Siddiqui, M. G. Fehlings, Spinal cord injuries: how could cell therapy help?, Expert Opinion on Biological Therapy 17(5) (2017) 529-541.
[3] N. N. Madigan, S. McMahon, T. O'Brien, M. J. Yaszemski, A. J. Windebank, Current tissue engineering and novel therapeutic approaches to axonal regeneration following spinal cord injury using polymer scaffolds, Respiratory physiology & neurobiology 169(2) (2009) 183-199.
[4] J. S. Hakim, M. Esmaeili Rad, P. J. Grahn, B. K. Chen, A. M. Knight, A. M. Schmeichel, N. A. Isaq, M. Dadsetan, M. J. Yaszemski, A. J. Windebank, Positively charged oligo [poly (ethylene glycol) fumarate] scaffold implantation results in a permissive lesion environment after spinal cord injury in rat, Tissue Engineering Part A 21(13-14) (2015) 2099-2114.
[5] B. K. Chen, N. N. Madigan, J. S. Hakim, M. Dadsetan, S. S. McMahon, M. J. Yaszemski, A. J. Windebank, GDNF Schwann cells in hydrogel scaffolds promote regional axon regeneration, remyelination and functional improvement after spinal cord transection in rats, Journal of Tissue Engineering and Regenerative Medicine (2017).
[6] G. M. Harris, N. N. Madigan, K. Z. Lancaster, L. W. Enquist, A. J. Windebank, J. Schwartz, J. E. Schwarzbauer, Nerve Guidance by a Decellularized Fibroblast Extracellular Matrix, Matrix Biology 60 (2017) 176-189.
[7] B. K. Chen, A. M. Knight, N. N. Madigan, L. Gross, M. Dadsetan, J. J. Nesbitt, G. E. Rooney, B. L. Currier, M. J. Yaszemski, R. J. Spinner, Comparison of polymer scaffolds in rat spinal cord: a step toward quantitative assessment of combinatorial approaches to spinal cord repair, Biomaterials 32(32) (2011) 8077-8086.
[8] M. Dadsetan, J. P. Szatkowski, M. J. Yaszemski, L. Lu, Characterization of photo-cross-linked Oligo [poly (ethylene glycol) fumarate] hydrogels for cartilage tissue engineering, Biomacromolecules 8(5) (2007) 1702-1709.
[9] M. Dadsetan, A. M. Knight, L. Lu, A. J. Windebank, M. J. Yaszemski, Stimulation of neurite Outgrowth using positively charged hydrogels, Biomaterials 30(23) (2009) 3874-3881.
[10] N. N. Madigan, B. K. Chen, A. M. Knight, G. E. Rooney, E. Sweeney, L. Kinnavane, M. J. Yaszemski, P. Dockery, T. O'Brien, S. S. McMahon, Comparison of cellular architecture, axonal growth, and blood vessel formation through cell-loaded polymer scaffolds in the transected rat spinal cord, Tissue Engineering Part A 20(21-22) (2014) 2985-2997.
[11] P. Clark, P. Connolly, A. Curtis, J. Dow, C. Wilkinson, Cell guidance by ultrafine topography in vitro, Journal of cell science 99(1) (1991) 73-77.
[12] L. Yao, S. Wang, W. Cui, R. Sherlock, C. O'Connell, G. Damodaran, A. Gorman, A. Windebank, A. Pandit, Effect of functionalized micropatterned PLGA on guided neurite growth, Acta Biomaterialia 5(2) (2009) 580-588.
[13] L. Yao, S. Wang, W. Cui, R. Sherlock, C. O'Connell, G. Damodaran, A. Gorman, A. Windebank, A. Pandit, Effect of functionalized micropatterned PLGA on guided neurite growth, Acta Biomater 5(2) (2009) 580-8.
[14] J. S. Goldner, J. M. Bruder, G. Li, D. Gazzola, D. Hoffman-Kim, Neurite bridging across micropatterned grooves, Biomaterials 27(3) (2006) 460-472.
[15] A. E. Haggerty, M. M. Marlow, M. Oudega, Extracellular matrix components as therapeutics for spinal cord injury, Neuroscience letters (2016).
[16] L. Yao, G. Damodaran, N. Nikolskaya, A. M. Gorman, A. Windebank, A. Pandit, The effect of laminin peptide gradient in enzymatically cross-linked collagen scaffolds on neurite growth, Journal of Biomedical Materials Research Part A 92(2) (2010) 484-492.
[17] D. N. Adams, E. Y. C. Kao, C. L. Hypolite, M. D. Distefano, W. S. Hu, P. C. Letourneau, Growth cones turn and migrate up an immobilized gradient of the laminin IKVAV peptide, Developmental Neurobiology 62(1) (2005) 134-147.
[18] A. Baron-Van Evercooren, H. K. Kleinman, H. Seppä, B. Rentier, M. Dubois-Dalcq, Fibronectin promotes rat Schwann cell growth and motility, The Journal of cell biology 93(1) (1982) 211-216.
[19] A. Mosahebi, M. Wiberg, G. Terenghi, Addition of fibronectin to alginate matrix improves peripheral nerve regeneration in tissue-engineered conduits, Tissue engineering 9(2) (2003) 209-218.
[20] C.-Y. Lin, Y.-S. Lee, V. W. Lin, J. Silver, Fibronectin inhibits chronic pain development after spinal cord injury, Journal of neurotrauma 29(3) (2012) 589-599.
[21] L. N. Novikova, L. N. Novikov, J.-O. Kellerth, Biopolymers and biodegradable smart implants for tissue regeneration after spinal cord injury, Current opinion in neurology 16(6) (2003) 711-715.
[22] A. M. Conti, S. J. Fischer, A. J. Windebank, Inhibition of axonal growth from sensory neurons by excess nerve growth factor, Annals of neurology 42(6) (1997) 838-846.
[23] L. E. Ta, L. Espeset, J. Podratz, A. J. Windebank, Neurotoxicity of oxaliplatin and cisplatin for dorsal root ganglion neurons correlates with platinum—DNA binding, Neurotoxicology 27(6) (2006) 992-1002.
[24] J. L. Podratz, A. J. Windebank, NGF rescues DRG neurons in vitro from oxidative damage produced by hemodialyzers, Neurotoxicology 26(3) (2005) 343-350.
[25] B. K. Chen, A. M. Knight, G. C. De Ruiter, R. J. Spinner, M. J. Yaszemski, B. L. Currier, A. J. Windebank, Axon regeneration through scaffold into distal spinal cord after transection, Journal of neurotrauma 26(10) (2009) 1759-1771.
[26] H. E. Olson, G. E. Rooney, L. Gross, J. J. Nesbitt, K. E. Galvin, A. Knight, B. Chen, M. J. Yaszemski, A. J. Windebank, Neural stem cell- and schwann cell-loaded biodegradable polymer scaffolds support axonal regeneration in the transected spinal cord, Tissue Engineering Part A 15(7) (2009) 1797-1805.
[27] J. Hakim, B. Rodysill, B. K. Chen, A. Schmeichel, M. J. Yaszemski, A. Windebank, N. Madigan, Combinatorial Tissue Engineering Partially Restores Function after Spinal Cord Injury, bioRxiv (2018) 254821.
[28] L. Liu, N. Wang, Y. Han, Y. Li, W. Liu, Redox-Triggered Self-Rolling Robust Hydrogel Tubes for Cell Encapsulation, Macromolecular Rapid Communications 35(3) (2014) 344-349.
[29] I. Vasiev, A. I. M. Greer, A. Z. Khokhar, J. Stormonth-Darling, K. E. Tanner, N. Gadegaard, Self-folding nano- and micropatterned hydrogel tissue engineering scaffolds by single step photolithographic process, Microelectronic Engineering 108 (2013) 76-81.
[30] Q. Zhang, J. Wommer, C. O'Rourke, J. Teitelman, Y. Tang, J. Robison, G. Lin, J. Yin, Origami and kirigami inspired self-folding for programming three-dimensional shape shifting of polymer sheets with light, Extreme Mechanics Letters 11 (2017) 111-120.
[31] H.1. Thérien-Aubin, Z. L. Wu, Z. Nie, E. Kumacheva, Multiple shape transformations of composite hydrogel sheets, Journal of the American Chemical Society 135(12) (2013) 4834-4839.
[32] M. Dadsetan, A. M. Knight, L. Lu, A. J. Windebank, M. J. Yaszemski, Stimulation of neurite outgrowth using positively charged hydrogels, Biomaterials 30(23-24) (2009) 3874-81.
[33] W. R. Gombotz, G. H. Wang, T. A. Horbett, A. S. Hoffman, Protein adsorption to poly(ethylene oxide) surfaces, J Biomed Mater Res 25(12) (1991) 1547-62.
[34] M. C. Dodla, R. V. Bellamkonda, Anisotropic scaffolds facilitate enhanced neurite extension in vitro, J Biomed Mater Res A 78(2) (2006) 213-21.
[35] G. M. Harris, N. N. Madigan, K. Z. Lancaster, L. W. Enquist, A. J. Windebank, J. Schwartz, J. E. Schwarzbauer, Nerve Guidance by a Decellularized Fibroblast Extracellular Matrix, Matrix Biol 60-61 (2017) 176-189.
[36] C. A. Sevilla, D. Dalecki, D. C. Hocking, Extracellular matrix fibronectin stimulates the self-assembly of microtissues on native collagen gels, Tissue Engineering Part A 16(12) (2010) 3805-3819.
[37] C. Deister, S. Aljabari, C. E. Schmidt, Effects of collagen 1, fibronectin, laminin and hyaluronic acid concentration in multi-component gels on neurite extension, Journal of Biomaterials Science, Polymer Edition 18(8) (2007) 983-997.
[38] C. Miller, S. Jeftinija, S. Mallapragada, Synergistic effects of physical and chemical guidance cues on neurite alignment and outgrowth on biodegradable polymer substrates, Tissue engineering 8(3) (2002) 367-78.
[39] Z. Ma, Z. Mao, C. Gao, Surface modification and property analysis of biomedical polymers used for tissue engineering, Colloids Surf B Biointerfaces 60(2) (2007) 137-57.
[40] S. G. Levesque, M. S. Shoichet, Synthesis of cell-adhesive dextran hydrogels and macroporous scaffolds, Biomaterials 27(30) (2006) 5277-85.
[41] M. C. Dodla, R. V. Bellamkonda, Differences between the effect of anisotropic and isotropic laminin and nerve growth factor presenting scaffolds on nerve regeneration across long peripheral nerve gaps, Biomaterials 29(1) (2008) 33-46.
[42] D. E. Koser, A. J. Thompson, S. K. Foster, A. Dwivedy, E. K. Pillai, G. K. Sheridan, H. Svoboda, M. Viana, L. da F Costa, J. Guck, Mechanosensing is critical for axon growth in the developing brain, Nature neuroscience 19(12) (2016) 1592.
[43] W.-L. Lei, S.-G. Xing, C.-Y. Deng, X.-C. Ju, X.-Y. Jiang, Z.-G. Luo, Laminin/β1 integrin signal triggers axon formation by promoting microtubule assembly and stabilization, Cell research 22(6) (2012) 954.
[44] M. E. Hatten, Riding the glial monorail: a common mechanism for glialguided neuronal migration in different regions of the developing mammalian brain, Trends in neurosciences 13(5) (1990) 179-184.
[45] A. M. Siddiqui, C. S. Ahuja, C. H. Tator, M. G. Fehlings, Chapter 3: Spinal cord protective and regenerative therapies, in: J. Jallo, A. Vaccaro (Eds.), Neurotrauma and Critical Care of the Spine; Second Edition, Thieme2018, p. 238.
[46] G. E. Rooney, A. M. Knight, N. N. Madigan, L. Gross, B. Chen, C. V. Giraldo, S. Seo, J. J. Nesbitt, M. Dadsetan, M. J. Yaszemski, Sustained delivery of dibutyryl cyclic adenosine monophosphate to the transected spinal cord via oligo [(polyethylene glycol) fumarate] hydrogels, Tissue Engineering Part A 17(9-10) (2011) 1287-1302.
[47] X. M. Xu, V. Guénard, N. Kleitman, M. B. Bunge, Axonal regeneration into Schwann cell-seeded guidance channels grafted into transected adult rat spinal cord, Journal of Comparative Neurology 351(1) (1995) 145-160.

The citation of any document or reference is not to be construed as an admission that it is prior art with respect to the present invention.

### Example 5

Mesenchymal stromal/stem cells (MSCs) are of a mesoderm origin that gives rise to osteocytes, adipocytes, and chondrocytes. MSC cell therapy has become popular due to their accessibility for autologous transplantation. Since MSCs can modulate the immune response, provide trophic support, and have angiogenic effects, they have become a popular choice in therapies for central nervous system (CNS) injury and disease. Much of the characterization of these cells has been done in culture and new research is routinely undertaken modify these cells in vitro. Most of this work has been done on plastic or glass surfaces, which does not reflect the surfaces encountered in the CNS. This study demonstrates that the response of MSCs to a soft and 3D environment differs than that of glass. Hydrogel sheets of oligo[poly(ethylene glycol)fumarate] were constructed with one side containing 100 μm high ridges. MSCs grown on the ridged side formed spheres, while on the flat side would characteristically lay down flat like on glass. However, on the softer polymer surface the MSCs have greater collagen IV deposited. Taken together, this emphasizes the importance of considering the surface structure and stiffness of where MSCs will be introduced when conducting informative in vitro studies.

Mesenchymal stromal/stem cells (MSCs) are a cell type of the mesoderm germ layer origin that can be isolated from many different tissue sources, such as adipose tissue, bone marrow, placenta, and umbilical cord. Since these sources are easy to isolate from adults, there is less of an ethical issue surrounding their use and presents an attractive autologous source. Being a multipotent cell, they have the ability to differentiate into osteoblasts, adipocytes, chondrocytes, and myocytes. Although these differentiated cell types may not be of interest in neuroscience, the MSC's angiogenic, trophic, and immune modulatory properties have been of great interest (Siddiqui, Ahuja et al. 2018). In a spinal cord injury model, it was demonstrated that MSCs from an umbilical cord source delivered through an intravenous route had a significant effect on circulating levels of anti-inflammatory cytokine interleukin 10 leading to better functional recovery, reduced hemorrhaging, and increased tissue sparing in the spinal cord (Badner, Siddiqui et al. 2017). Preclinical results using MSCs have been so promising that a search for mesenchymal stromal cells or mesenchymal stem cells and spinal cord yields 18 active clinical trials on clinicaltrails.gov. It has been demonstrated that intrathecal administration of autologous adipose derived MSCs in 72 amyotrophic lateral sclerosis (ALS) patients was safe with only temporary pain in some patients and possibly some anecdotal mild clinical improvements in a phase I dose escalation safety trail at the Mayo Clinic (Staff, Madigan et al. 2016).

Although there has been great advances in studying the use of MSCs in disease, it is not fully understood which mechanisms are important nor the full extent to the secretory profile of the cells. Another issue has been how to enhance the efficacy and survival of these cells to provide more robust therapeutic effects in patients. To elucidate many of these questions, in vitro studies have been widely used. However, question remains how closely this in vitro environment mimics the in vivo condition and likely many of the findings are not translatable due to this difference. The most common way to study MSCs in vitro is by the use of plastic or glass culture dishes. In fact, the isolation of MSCs is highly dependent on its ability to adhere to plastic. However, most organs and the central nervous system are not stiff 2D structures. Many have advocated for the use biomaterials and 3D culturing techniques to best mimic the physical properties target organs. A recently study demonstrated gene expression changes of MSCs on hydrogels that were stiffness dependent (Darnell, Gu et al. 2018). In addition, cytokine secretion profiles, such of that of interleukin 6, could be affected by the surface structure (Leuning, Beijer et al. 2018).

In this study, we present the interaction of MSCs with hydrogel biomaterials composed of positively charged oligo[poly(ethylene glycol)fumarate] (OPF+) constructed as a 0.08 mm thick sheet with ridges spaced 0.4 mm apart. When hydrated, OPF+ has consistency similar to that of the spinal cord where the compression and flexural modulus is not statistically different (Chen, Knight et al. 2011). OPF+ sheet can be rolled and inserted into the transected spinal cord where it integrates and supports axonal growth (Siddiqui, Brunner et al. 2019). The OPF+ sheet has also been previous used in vitro where it was shown that the ridges act to align neurons and Schwann cells and the addition of an extracellular matrix protein coat would aid neurite elongation (Siddiqui, Brunner et al. 2019). OPF+ in this ridged sheet design has great versatility for in vitro and in vivo studies of the spinal cord.

OPF was synthesized as previous described (Dadsetan, Szatkowski et al. 2007, Dadsetan, Szatkowski et al. 2007, Dadsetan, Knight et al. 2009). OPF+ ridged sheets were fabricated as previously described in (Siddiqui, Brunner et al. 2019). Briefly, a Teflon mold with microgrooves 100 μm deep and 0.4 mm apart had OPF+ liquid polymer pipetted on in between a 0.08 mm thick spacer. After polymerization by exposure to ultraviolet light (365 nm) for one hour, the material was cured overnight. The resulting ridged scaffold was cut in 2 mm lengths for culture.

Human MSCs (hMSCs) were isolated from adipose tissue biopsies and then cultured, expanded, and cryopreserved by the Mayo Clinic Human Cellular Therapy Laboratory as previously described in Staff et al. (2016). The release criteria included culture sterility, mycoplasma testing, cytogenetic analysis as well as cellular phenotyping including CD90+, CD105+. CD73+, HLA Class I+, CD14+, CD44+, HLA DR, and CD45-. The cells were cultured in advance MEM media (Gibco) supplemented with human platelet lysate (5%; Lawson, Mill Creek), 100 U/mL Antibiotic-Antimycotic (Gibco), 100 units/mL Glutamax (Gibco), and 1000 USP units heparin (Novaplus) at 5% CO2 and 37° C.

Rat MSCs were isolated from femur and tibia bone marrow from Sprague Dawley rats as previously described (Rooney, Moran et al. 2008). They were cultured in in Dulbecco's Modified Eagle Medium F12 medium (DMEM/F12; Gibco) supplemented with 10% fetal bovine serum, and 100 U/mL Antibiotic-Antimycotic (Gibco) at 5% CO2 and 37° C.

Rat Schwann cells were harvested from the sciatic nerve of 2-5 day of Sprague Dawley pup as previously described (Chen, Knight et al. 2009, Olson, Rooney et al. 2009). The cells were cultured in DMEM/F12 containing 10% fetal bovine serum, 100 U/mL Antibiotic-Antimycotic, 2 μM Forskolin (Sigma), 10 ng/mL neurgulinin-1 at 5% CO2 and 37° C.

OPF+ was hydrated in culture media was pinned to sterile culture dishes containing SYLGARD 184 Silicone Elastomer (Dow Corning) to prevent spontaneous rolling of the scaffold. The plate were sterilized with 20 minute exposure to UV and multiple ethanol washes before pinning, while the scaffold sheets were sterilized using multiple ethanol washes. The OPF+ sheets were coated with either media only, poly-1-lysine (PLL; Sigma-Aldrich; 0.01%; 5 minutes), laminin (100 μg/ml; Sigma-Aldrich), or rat plasma fibronectin (10 μg/ml) at 37° C. overnight. Fibronectin was isolated from rat serum using gelatin-Sepharose affinity chromatography as previously described (Harris, Madigan et al. 2017). Excess ECM protein coat or PLL was removed with 3 washes with culture media specific to the cell type that would be plated. Once the cells were 70-80% confluent, 150,000 cells were seeded onto each OFP+ sheet for 7 days. Three sheets per condition were used. The OPF+ sheets with the cells were fixed using 4% paraformaldehyde in phosphate buffered saline for 20 minutes. Three (3) washes with PBS were done to remove excess PFA and then immunocytochemistry was performed.

Non-specific binding sites were blocked using a solution containing PBS, 10% normal donkey serum, and 0.3% Triton X-100 for 30 minutes. Then scaffolds containing cells were incubated with mouse a collagen IV primary antibody (1:800; Millipore) overnight at 4° C. Then the samples were washed 3 times with PBS and then incubated for 1 hour with the donkey anti-mouse Cy3; 1:200; Millipore Chemicon) and phalloidin conjugated to Alexa 488 (1:100; Molecular Probes) at room temperature. Then the samples were washed 3 times in PBS before being placed on a slide and coverslip in SlowFade Gold containing DAPI (Invitrogen). In the case of cells grown on coverslips, they were inverted onto a glass slide with SlowFade Gold containing DAPI following the same procedure above.

Fluorescent images were acquired using an inverted fluorescence microscope Zeiss Axio Observer Z-1 with a motorized stage mounted with an Axiocam 503 camera (Carl Zeiss). Zen 2 (blue edition; Carl Zeiss) software was used to acquire and process the images. Z-stacks with approximately 5 slides were taken at 20×. Bright field images of live cultures were taken on a Zeiss Axiovert Model 35 microscope with a Nikon CCD camera at 10× magnification. On each OPF+ sheet or coverslip, 4 representative areas were taken in a non-bias manner by moving one field of view up or down from a central position and 2 fields of view across. Three different replicates were used for each condition (3 scaffolds or coverslips).

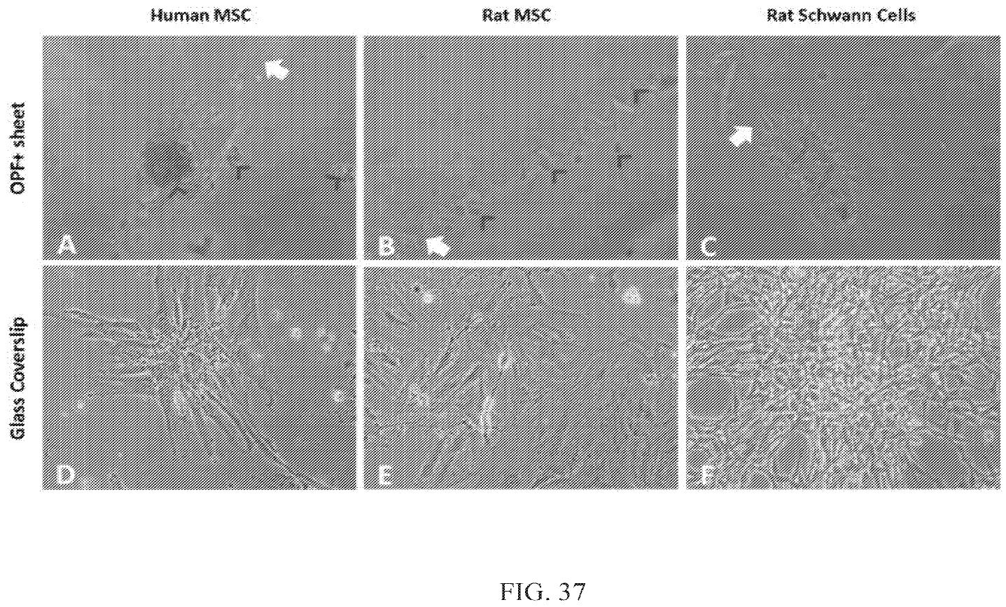

Human MSCs, rat MSCs, and rat Schwann cells were grown on the ridged OPF+ sheet and glass coverslips (FIG. 1). At 3 days in culture, the cells are found on or around the ridges on the OPF+ sheet (FIG. 37A-1C). However, the human or rat MSCs do not adhere flat on the surface of the polymer (FIGS. 37A and 1B; white arrow indicates the ridge, while arrowheads indicates spheres) as the rat Schwann cells do. Interestingly, the rat MSCs form smaller spheres where the human MSCs form fewer larger spheres. Furthermore, when the cells are grown on glass coverslips (FIG. 37D-1F) they exhibit their normal, stereotypical shape and are adhered flat.

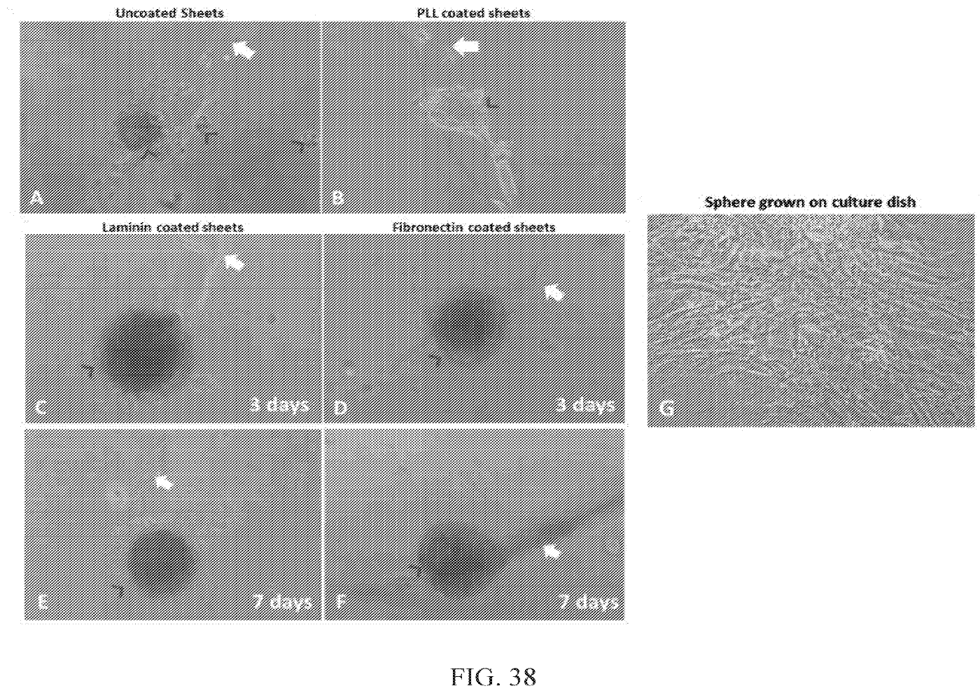

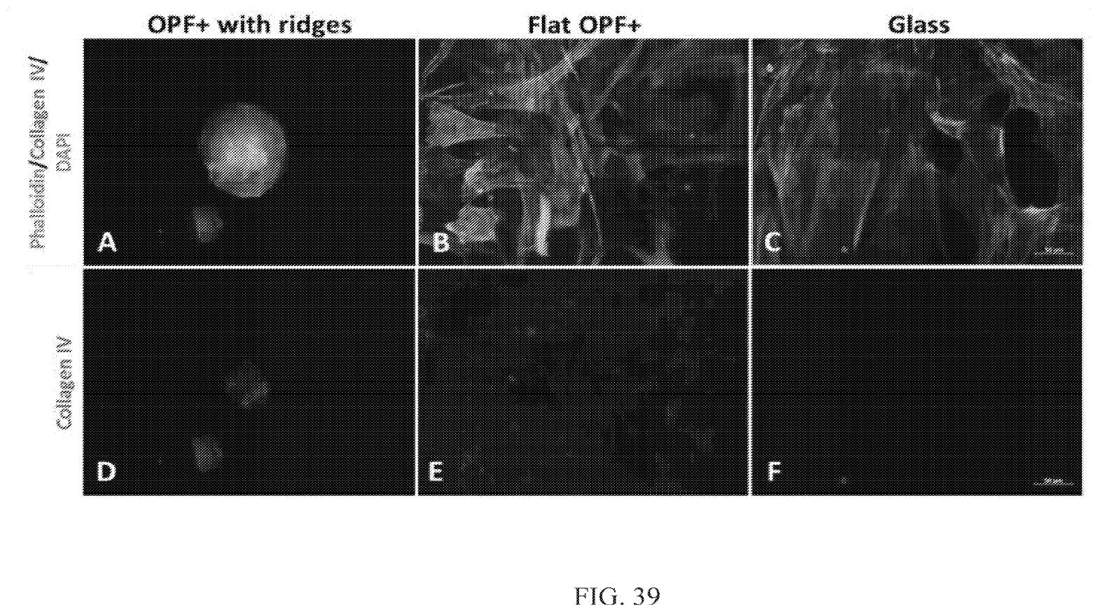

Since these OPF+ sheets were not coated with any extracellular matrix molecules other than those found in the media, we coated the OPF+ ridged sheets with PLL, laminin, or fibronectin protein coats. Again, we found that the human MSCs were found mostly on the ridges in a sphere (FIG. 38A-38D) 3 days in culture. Even after 7 days, the MSCs do not lay flat and continue to grow in a sphere formation (FIGS. 38E and 38F). To test if these spheres were viable and exhibit normal morphology, they were picked and plated in normal culture dishes (FIG. 38G). The spheres were shown to flatten down on the culture dish and grow normally.

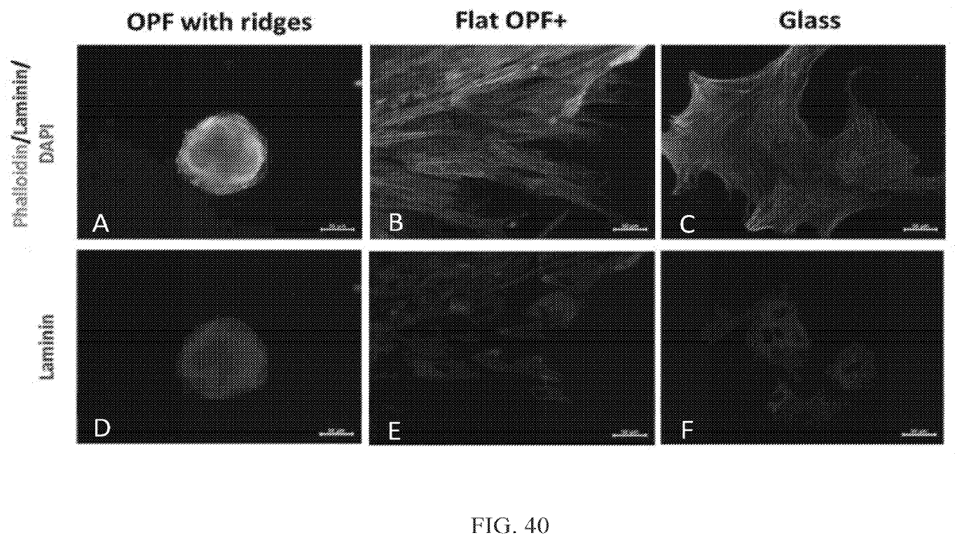

Next we wanted to test if this behavior of the cells become spheres was due to the material being softer than plastic/glass or due to the 3D shape formed by the 0.1 mm high ridges. When human MSCs are grown on the flat side of the OPF+ scaffold, we found that the cells adhered to the material in a flat manner like on glass and plastic (FIG. 39B). Staining for actin with phallodin, we show that the cytoskeletal structure looks similar to that of human MSCs grown on glass coverslips (FIG. 39C). Interestingly, collagen IV was present when human MSCs grown on the softer OPF+ material (FIGS. 39D and 39E) but not when grown on the glass coverslip (FIG. 39F) after 7 days in culture. When the human MSCs were in a sphere on the ridged OPF+, the collagen IV is present within the sphere (FIGS. 39A and 39D) whereas when grown on the flat OPF+ sheet it is more globally present under and near the cells (FIGS. 39B and 39E). However, staining for laminin (FIG. 40) did not show a difference between the flat OPF+(FIG. 40E) and glass (FIG. 40F) but was found in the sphere when grown on OPF+ with ridges (FIG. 40D).

This study demonstrates MSCs preferentially adhere to ridged surfaces on OPF+, instead of the adjacent flat area, however forming spheres. On flat OPF+ sheets, glass, or plastic, MSCs will take their stereotypical flat morphology. The addition of ECM protein coats did not aid in flat attachment to the ridged OPF+ scaffold. In addition, on the softer, spinal cord like OPF+ material, the MSCs produced collagen IV. Taken together, this demonstrates the importance of the stiffness and physical structure of surfaces where the MSCs are expected to adhere or engraft.

MSCs are a popular cell type for use in CNS injuries and disease. They can provide trophic support, modulate the immune system, and promote angiogenesis (Badner, Siddiqui et al. 2017). However, studies have demonstrated that they do not survive or engraft for long periods of time in the spinal cord (Himes, Neuhuber et al. 2006, Abrams, Dominguez et al. 2009, Shin, Kim et al. 2013). When used for treatments for CNS injures and disorders, they are injected into or on top of soft surfaces such as the brain or spinal cord. However, many of the studies that are done to characterize MSCs in vitro are done on glass or plastic surfaces (Schinkothe, Bloch et al. 2008, Buron, Perrin et al. 2009, Redondo-Castro, Cunningham et al. 2017). In this study we demonstrate that cell characteristics can change depending on surface morphology and stiffness. Gene expression changes, cell adhesion properties, and changes in proliferation have been previously described being influenced by surface structure and stiffness that the MSCs are grown on top (Leuning, Beijer et al. 2018, Sun, Chi et al. 2018). Stiffer material promoted adhesion and proliferation of bone marrow derived MSCs and surfaces at the range of 62-68 kPA could push osteogenic differentiation (Sun, Chi et al. 2018). We found that our soft OPF+ surface had greater amounts of collagen IV than MSCs grown on glass. This could indicate either a difference in ECM production due to different microenvironment cues or beginnings of adipogenic differentiation (Sillat, Saat et al. 2012).

How closely the in vitro environment mimics the in vivo one has real clinical implications as well. Staff et al. (2016) found that as the dose of the MSCs increased, more patients were seen with lumbosacral nerve root thickening with gadolinium enhancement on Mill. This resembled clumping and was like arachnoiditis, however on autopsy of 4 patients that died during the study of ALS did not reveal signs of tumor formation or arachnoiditis (Staff, Madigan et al. 2016). One question that remains is if this nodular like structure on the nerve root could have been due to MSCs accumulating or formation as spheres on this soft and 3D surface with ridges and grooves. Although the administration of MSCs is safe and well tolerated, this example highlights the importance of investigating cell behavior on surfaces akin to the site of injection.

Use of 3D culturing methods and hydrogel scaffolds to mimic surface structures can be informative when tailoring treatments for certain disorders. OPF is an interesting choice of polymer since it can be modified to from different shapes, sizes, and stiffnesses (Temenoff, Athanasiou et al. 2002, Shin, Quinten Ruhe et al. 2003, Dadsetan, Szatkowski et al. 2007, Dadsetan, Knight et al. 2009, Hakim, Esmaeili Rad et al. 2015). Aside from having in vivo applications (Chen, Knight et al. 2011, Madigan, Chen et al. 2014, Hakim, Esmaeili Rad et al. 2015, Chen, Madigan et al. 2017, Hakim, Rodysill et al. 2019), this study demonstrates a further application for OPF+ hydrogel sheets for in vitro investigation as they are easy to culture on top and can be constructed into different stiffnesses with different surface topology

### REFERENCES FOR EXAMPLE 5

1. Abrams, M. B., C. Dominguez, K. Pernold, R. Reger, Z. Wiesenfeld-Hallin, L. Olson and D. Prockop (2009). “Multipotent mesenchymal stromal cells attenuate chronic inflammation and injury-induced sensitivity to mechanical stimuli in experimental spinal cord injury.” Restorative neurology and neuroscience 27(4): 307-321.
2. Badner, A., A. M. Siddiqui and M. G. Fehlings (2017). “Spinal cord injuries: how could cell therapy help?” Expert Opin Biol Ther 17(5): 529-541.
3. Buron, F., H. Perrin, C. Malcus, O. Héquet, O. Thaunat, M. N. Kholopp-Sarda, F. T. Moulin and E. Morelon (2009). “Human Mesenchymal Stem Cells and Immunosuppressive Drug Interactions in Allogeneic Responses: An In Vitro Study Using Human Cells.” Transplantation Proceedings 41(8): 3347-3352.
4. Chen, B. K., A. M. Knight, G. C. W. de Ruiter, M. J. Yaszemski, B. L. Currier and A. J. Windebank (2009). “Axon regeneration through scaffold into distal spinal cord after transection.” J Neurotrauma 26(10): 1759-1771.
5. Chen, B. K., A. M. Knight, N. N. Madigan, L. Gross, M. Dadsetan, J. J. Nesbitt, G. E. Rooney, B. L. Currier, M. J. Yaszemski, R. J. Spinner and A. J. Windebank (2011). “Comparison of polymer scaffolds in rat spinal cord: A step toward quantitative assessment of combinatorial approaches to spinal cord repair.” Biomaterials 32: 8077-8086.
6. Chen, B. K., N. N. Madigan, J. S. Hakim, M. Dadsetan, S. S. McMahon, M. J. Yaszemski and A. J. Windebank (2017). “GDNF Schwann cells in hydrogel scaffolds promote regional axon regeneration, remyelination and functional improvement after spinal cord transection in rats.” J Tissue Eng Regen Med.
7. Dadsetan, M., A. M. Knight, L. Lu, A. J. Windebank and M. J. Yaszemski (2009). “Stimulation of neurite outgrowth using positively charged hydrogels.” Biomaterials 30(23-24): 3874-3881.
8. Dadsetan, M., J. P. Szatkowski, M. J. Yaszemski and L. Lu (2007). “Characterization of photo-cross-linked oligo [poly (ethylene glycol) fumarate] hydrogels for cartilage tissue engineering.” Biomacromolecules 8(5): 1702-1709.
9. Dadsetan, M., J. P. Szatkowski, M. J. Yaszemski and L. Lu (2007). “Characterization of photo-cross-linked oligo[poly(ethylene glycol) fumarate] hydrogels for cartilage tissue engineering.” Biomacromolecules 8(5): 1702-1709.
10. Darnell, M., L. Gu and D. Mooney (2018). “RNA-seq reveals diverse effects of substrate stiffness on mesenchymal stem cells.” Biomaterials 181: 182-188.
11. Hakim, J. S., M. Esmaeili Rad, P. J. Grahn, B. K. Chen, A. M. Knight, A. M. Schmeichel, N. A. Isaq, M. Dadsetan, M. J. Yaszemski and A. J. Windebank (2015). “Positively Charged Oligo[Poly(Ethylene Glycol) Fumarate] Scaffold Implantation Results in a Permissive Lesion Environment after Spinal Cord Injury in Rat.” Tissue Eng Part A 21(13-14): 2099-2114.
12. Hakim, J. S., B. R. Rodysill, B. K. Chen, A. M. Schmeichel, M. J. Yaszemski, A. J. Windebank and N. N. Madigan (2019). “Combinatorial Tissue Engineering Partially Restores Function after Spinal Cord Injury.” Journal of tissue engineering and regenerative medicine.
13. Harris, G. M., N. N. Madigan, K. Z. Lancaster, L. W. Enquist, A. J. Windebank, J. Schwartz and J. E. Schwarzbauer (2017). “Nerve Guidance by a Decellularized Fibroblast Extracellular Matrix.” Matrix Biol 60-61: 176-189.
14. Himes, B. T., B. Neuhuber, C. Coleman, R. Kushner, S. A. Swanger, G. C. Kopen, J. Wagner, J. S. Shumsky and I. Fischer (2006). “Recovery of function following grafting of human bone marrow-derived stromal cells into the injured spinal cord.” Neurorehabilitation and neural repair 20(2): 278-296.
15. Leuning, D. G., N. R. Beijer, N. A. Fosse, S. Vermeulen, E. Lievers, C. Kooten, T. J. Rabelink and J. de Boer (2018). “The cytokine secretion profile of mesenchymal stromal cells is determined by surface structure of the microenvironment.” Scientific reports 8(1): 7716.
16. Madigan, N. N., B. K. Chen, A. M. Knight, G. E. Rooney, E. Sweeney, L. Kinnavane, M. J. Yaszemski, P. Dockery, T. O'Brien, S. S. McMahon and A. J. Windebank (2014). “Comparison of Cellular Architecture, Axonal Growth, and Blood Vessel Formation Through Cell-Loaded Polymer Scaffolds in the Transected Rat Spinal Cord.” Tissue Eng Part A 20: 2985-2997.
17. Olson, H. E., G. E. Rooney, L. Gross, J. J. Nesbitt, K. E. Galvin, A. Knight, B. Chen, M. J. Yaszemski and A. J. Windebank (2009). “Neural stem cell- and schwann cell-loaded biodegradable polymer scaffolds support axonal regeneration in the transected spinal cord.” Tissue Eng Part A 15(7): 1797-1805.
18. Redondo-Castro, E., C. Cunningham, J. Miller, L. Martuscelli, S. Aoulad-Ali, N. J. Rothwell, C. M. Kielty, S. M. Allan and E. Pinteaux (2017). “Interleukin-1 primes human mesenchymal stem cells towards an anti-inflammatory and pro-trophic phenotype in vitro.” Stem Cell Research & Therapy 8(1): 79.
19. Rooney, G. E., C. Moran, S. S. McMahon, T. Ritter, M. Maenz, A. Flugel, P. Dockery, T. O'Brien, L. Howard, A. J. Windebank and F. P. Barry (2008). “Gene-modified mesenchymal stem cells express functionally active nerve growth factor on an engineered poly lactic glycolic acid (PLGA) substrate.” Tissue Eng Part A 14(5): 681-690.
20. Schinkothe, T., W. Bloch and A. Schmidt (2008). “In Vitro Secreting Profile of Human Mesenchymal Stem Cells.” Stem Cells and Development 17(1): 199-206.
21. Shin, D. A., J.-M. Kim, H.-I. Kim, S. Yi, Y. Ha, D. H. Yoon and K. N. Kim (2013). “Comparison of functional and histological outcomes after intralesional, intracisternal, and intravenous transplantation of human bone marrow-derived mesenchymal stromal cells in a rat model of spinal cord injury.” Acta Neurochirurgica 155(10): 1943-1950.
22. Shin, H., P. Quinten Ruhe, A. G. Mikos and J. A. Jansen (2003). “In vivo bone and soft tissue response to injectable, biodegradable oligo(poly(ethylene glycol) fumarate) hydrogels.” Biomaterials 24(19): 3201-3211.
23. Siddiqui, A. M., C. S. Ahuja, C. H. Tator and M. G. Fehlings (2018). Chapter 3: Spinal cord protective and regenerative therapies. Neurotrauma and Critical Care of the Spine; Second Edition. J. Jallo and A. Vaccaro, Thieme: 238.
24. Siddiqui, A. M., R. Brunner, G. M. Harris, A. L. Miller, B. E. Waletzki, J. E. Schwarzbauer, J. Schwartz, M. J. Yaszemski, A. J. Windebank and N. N. Madigan (2019). “Promoting neuronal outgrowth using ridged scaffolds coated with extracellular matrix proteins.” bioRxiv: 788539.
25. Sillat, T., R. Saat, R. Pöllänen, M. Hukkanen, M. Takagi and Y. T. Konttinen (2012). “Basement membrane collagen type IV expression by human mesenchymal stem cells during adipogenic differentiation.” Journal of Cellular and Molecular Medicine 16(7): 1485-1495.
26. Staff, N. P., N. N. Madigan, J. Morris, M. Jentoft, E. J. Sorenson, G. Butler, D. Gastineau, A. Dietz and A. J. Windebank (2016). “Safety of intrathecal autologous adipose-derived mesenchymal stromal cells in patients with ALS.” Neurology 87(21): 2230-2234.
27. Sun, M., G. Chi, P. Li, S. Lv, J. Xu, Z. Xu, Y. Xia, Y. Tan, J. Xu, L. Li and Y. Li (2018). “Effects of Matrix Stiffness on the Morphology, Adhesion, Proliferation and Osteogenic Differentiation of Mesenchymal Stem Cells.” International journal of medical sciences 15(3): 257-268.
28. Temenoff, J. S., K. A. Athanasiou, R. G. LeBaron and A. G. Mikos (2002). “Effect of poly(ethylene glycol) molecular weight on tensile and swelling properties of oligo(poly(ethylene glycol) fumarate) hydrogels for cartilage tissue engineering.” J Biomed Mater Res 59(3): 429-437.

The citation of any document or reference is not to be construed as an admission that it is prior art with respect to the present invention.

Thus, the invention provides a chemically patterned matrix for neural regeneration.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

1. A matrix for neuron regeneration, the matrix comprising:
a sheet having a first surface and a second surface opposite the first surface, the second surface having a plurality of integrally formed ridges;
wherein the sheet has a spiral shape, such that the first surface of the sheet faces the second surface of the sheet; and
wherein the sheet including the integrally formed ridges, comprises oligo(poly(ethylene glycol) fumarate).

2. The matrix of claim 1, wherein the first surface includes a patterned surface, the patterned surface including a plurality of channels; and
wherein the plurality of channels are substantially parallel.

3. The matrix of claim 2, wherein each of the plurality of channels have a width and a depth;
wherein the width is in a range of 10 μm to 80 μm; and
wherein the depth is in a range of 10 nm to 70 nm.

4. The matrix of claim 1, wherein the first surface includes a metal oxide layer, the metal oxide layer covering at least a portion of the second surface, and wherein the metal oxide layer includes a plurality of channels directed into the metal oxide layer, the plurality of channels being substantially parallel to each other.

5. The matrix of claim 4, wherein the metal oxide layer comprises a material selected from the group consisting of titanium dioxide, zirconium dioxide, and combinations thereof.

6. The matrix of claim 4, further comprising a self-assembled alkylphosphonate monolayer disposed on at least a portion of the metal oxide layer.

7. The matrix of claim 6, wherein the alkylphosphonate monolayer is disposed on the metal oxide layer, and
wherein the alkylphosphonate monolayer further defines the plurality of channels, such that a height of each channel is defined by a height of the metal oxide layer and a height of the alkylphosphonate monolayer.

8. The matrix of claim 7, wherein the metal oxide layer and the alkylphosphonate monolayer defines a patterned surface, and
wherein the plurality of channels extend through the entirety of the patterned surface, such that the first surface of the sheet lines a bottom surface of each channel.

9. The matrix of claim 6, further comprising Schwann cells disposed on the alkylphosphonate monolayer, or within the plurality of channels.

10. The matrix of claim 9, further comprising human mesenchymal stromal cells disposed on the alkylphosphonate monolayer, or within the plurality of channels.

11. The matrix of claim 10, wherein the human mesenchymal stromal cells, or the Schwann cells are modified to secrete glial cell-derived neurotrophic factor.

12. The matrix of claim 10, wherein the plurality of channels are configured to orient a growth of cells along the channel.

13. (canceled)

14. (canceled)

15. (canceled)

16. A matrix for neuron regeneration, the matrix comprising:
a sheet having a first surface and a second surface opposite the first surface, the second surface having a plurality of ridges;
a metal oxide layer disposed on at least a portion of the first surface;
a self-assembled alkylphosphonate monolayer disposed on at least a portion of the metal oxide layer;
wherein the sheet has a spiral shape, such that the first surface of the sheet faces the second surface of the sheet; and
wherein the sheet comprises a hydrogel.

17. (canceled)

18. (canceled)

19. (canceled)

20. (canceled)

21. (canceled)

22. (canceled)

23. (canceled)

24. The matrix of claim 16, wherein the plurality of ridges are longitudinally spaced apart from each other at a first distance.

25. The matrix of claim 24, wherein the first distance is in a range of 0.1 mm to 2.0 mm.

26. The matrix of claim 24, wherein the plurality of ridges are laterally spaced apart from each other at a second distance.

27. (canceled)

28. (canceled)

29. (canceled)

30. (canceled)

31. (canceled)

32. (canceled)

33. (canceled)

34. (canceled)

35. (canceled)

36. A matrix for neural regeneration comprising:
a sheet having:
a first surface having a plurality of channels, the plurality of channels being substantially parallel to each other, and
a second surface opposite the first surface and having a plurality of ridges; and
wherein the sheet is rolled into a spiral configuration about an axis, such that the plurality of channels extend along the axis, and a portion of the first surface faces a portion of the second surface.

37. The matrix of claim 36, wherein the channels are defined between adjacent strips of metal oxide layers, the metal oxide layers having a self-assembling monolayer disposed thereon.

38. The matrix of claim 36, wherein each channel within the plurality of channels has a width, and wherein the width is in a range between 10 μm and 80 μm.

39. The matrix of claim 36, wherein the channels are directed into the first surface of the sheet, such that the first surface of the sheet defines the channels.

40. (canceled)

41. (canceled)

42. (canceled)

43. (canceled)

44. (canceled)

45. (canceled)
